SEQUENCE LISTING (29 sequences)

SEQ ID NO: 1 - DNA, 232 bp, primate
ttggccttagggaccaagcttttatcatcgtcagtgggacttaacctgtcttaaaagtgctgcttctcctacactcgctcaagatcccgagtcagctgtattatggcatcctattagtcaggcagcctgtgcttcaagcccgtagttgtattcatcccctaaaggggccattccgtttgtatcatcacatgtcctcagtgggtccatgtgtatatcaaggacatgatgcaga

SEQ ID NO: 2 - protein, 77 aa, primate
  SITE (18): Xaa at 18, 39, 54;  sequence problem
LALGTKLLSSSVGLNLSXKCCFSYTRSRSRVSCIMASYXSGSLCFKPVVVFIPXRGHSVCIITCPQWVHVYIKDMMQ

SEQ ID NO: 3 - protein, 72 aa, primate
  SITE (50): sequence problem
TKTESSSRGPYHPSECCFTYTTYKIPRQRIMDYYETNSQCSKPGIVFITXRGHSVCTNPSDKWVQDYIKDMK

SEQ ID NO: 4 - protein, 143 aa, primate
  SITE (143): Xaa due to sequence problem
MKISVAAIPFFLLITIALGTKTESSSRGPYHPSECCFTYTTYKIPRQRIMDYYETNSQCSKPGIVFITKRGHSVCTNPSDKWVQDYIKDMKENTKTESSSRGPYHPSECCFTYTTYKIPRQRIMDYYETNSQCSKPGIVFITX

SEQ ID NO: 5 - protein, 93 aa, primate
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 6 - protein, 93 aa, primate; identical to SEQ ID NO: 5

SEQ ID NO: 7 - protein, 93 aa, primate; identical to SEQ ID NO: 5

SEQ ID NO: 8 - DNA, 1082 bp, primate
  CDS (1)..(1080)
  misc_feature (20): nucleotide may be G
atgcctttccccggcccacrcgcaggtagatcttccactctaaaggacaccacccntccatcccaccaaatatttggaaggctcctggaagatctccaaatcnaagtgtctcccactgcccacggcattycagacacttttgacccttacctggacatcgccctggatatccaggcagctcagagtgtcmagcaagctttggaacagttggtgaagcccgaagaactcaatggagagaatgcctatcattgtggtstttgtctccagagggcgccggcctccaagacgttaactttacacacctctgccaaggtcctcatccttgtcttgaagagattctccgatgtcacaggcaacaagattgccaagaatgtgcaatatcctgagtgccttgacatgcagccatacatgtctcagcagaacacaggacctcttgtctatgtcctctatgctgtgctggtccacgctgggtggagttgtcacaacggacattacttctcttatgtcaaagctcaagaaggccagtggtataaaatggatgatgccgaggtcaccgcctctagcatcacttctgtcctgagtcaacaggcctacgtcctcttttacatccagaagagtgaatgggaaagacacagtgagagtgtgtcaagaggcagggaaccaagagcccttggcgcagaagacacagacaggcgagcaacgcaaggagagctcaagagagaccacccctgcctccaggcccccgagttggacgagcacttggtggaaagagccactcaggaaagcaccttagaccactggaaattccttcaagagcaaaacaaaacgaagcctgagttcaacgtcagaaaagtcgaaggtaccctgcctcccgacgtacttgtgattcatcaatcaaaatacaagtgtgggatgaagaaccatcatcctgaacagcaaagctccctgctaaacctctcttcgacgaccccgacacatcaggagtccatgaacactggcacactcgcttccctgcgagggagggccaggagatccaaagggaagaacaaacacagcaagagggctctgcttgtgtgccagtg

SEQ ID NO: 9 - protein, 360 aa, primate
MPFPGPXAGRSSTLKDTTXPSHQIFGRLLEDLQIXVSPTAHGIXDTFDPYLDIALDIQAAQSVXQALEQLVKPEELNGENAYHCGXCLQRAPASKTLTLHTSAKVLILVLKRFSDVTGNKIAKNVQYPECLDMQPYMSQQNTGPLVYVLYAVLVHAGWSCHNGHYFSYVKAQEGQWYKMDDAEVTASSITSVLSQQAYVLFYIQKSEWERHSESVSRGREPRALGAEDTDRRATQGELKRDHPCLQAPELDEHLVERATQESTLDHWKFLQEQNKTKPEFNVRKVEGTLPPDVLVIHQSKYKCGMKNHHPEQQSSLLNLSSTTPTHQESMNTGTLASLRGRARRSKGKNKHSKRALLVCQ

SEQ ID NO: 10 - DNA, 1683 bp, primate
  CDS (1)..(1590)
  misc_feature (837): nucleotide 837 may be a or c; resulting amino acid depends upon genetic code
atggaggacgactcactctacttgggaggtgagtggcagttcaaccacttttcaaaactcacatcttctcggccagatgcagcttttgctgaaatccagcggacttctctccctgagaagtcaccactctcatctgaggcccgtgtcgacctctgtgatgatttggctcctgtggcaagacagcttgctcccagggagaagcttcctctgagtagcaggagacctgctgcggtgggggctgggctccagaatatgggaaatacctgctacgagaacgcttccctgcagtgcctgacatacacaccgccccttgccaactacatgctgtcccgggagcactctcaaacatgtcagcgtcccaagtgctgcatgctctgtactatgcaagctcacatcacatgggccctccacagtcctggtcatgtcatccagccctcacaggcattggctgctggcttccatagaggcaagcaggaagatgcccatgaatttctcatgttcactgtggatgccatgaaaaaggcatgccttcccggccacaagcaggtagatcatcactctaaggacaccaccctcatccaccaaatatttggaggctgctggagatctcaaatcaagtgtctccactgccacgggattccagacacttttgacccttacctggacatcgccctggatatccaggcagctcagagtgtcaagcaagctttggaacagttggtgaagcccgaagaactcaatggagagaatgcctatcattgtggtctttgtctccagagggcgccggcctccaagacgttaactttacacacttctgccaaggtcctcatccttgtmttgaagagattctccgatgtcacaggcaacaaacttgccaagaatgtgcaatatcctgagtgccttgacatgcagccatacatgtctcagcagaacacaggacctcttgtctatgtcctctatgctgtgctggtccacgctgggtggagttgtcacaacggacattacttctcttatgtcaaagctcaagaaggccagtggtataaaatggatgatgccgaggtcaccgcctctagcatcacttctgtcctgagtcaacaggcctacgtcctcttttacatccagaagagtgaatgggaaagacacagtgagagtgtgtcaagaggcagggaaccaagagcccttggcgcagaagacacagacaggcgagcaacgcaaggagagctcaagagagaccacccctgcctccaggcccccgagttggacgagcacttggtggaaagagccactcaggaaagcaccttagaccactggaaattccttcaagagcaaaacaaaacgaagcctgagttcaacgtcagaaaagtcgaaggtaccctgcctcccgacgtacttgtgattcatcaatcaaaatacaagtgtgggatgaagaaccatcatcctgaacagcaaagctccctgctaaacctctcttcgacgaccccgacacatcaggagtccatgaacactggcacactcgcttccctgcgagggagggccaggagatccaaagggaagaacaaacacagcaagagggctctgcttgtgtgccagtgatctcagtggaagtaccgacccacacgtaggggtgcacacacacacgcacacacacagacacacacataactacacccagaagcgcgctga

SEQ ID NO: 11 - protein, 530 aa, primate
MEDDSLYLGGEWQFNHFSKLTSSRPDAAFAEIQRTSLPEKSPLSSEARVDLCDDLAPVARQLAPREKLPLSSRRPAAVGAGLQNMGNTCYENASLQCLTYTPPLANYMLSREHSQTCQRPKCCMLCTMQAHITWALHSPGHVIQPSQALAAGFHRGKQEDAHEFLMFTVDAMKKACLPGHKQVDHHSKDTTLIHQIFGGCWRSQIKCLHCHGIPDTFDPYLDIALDIQAAQSVKQALEQLVKPEELNGENAYHCGLCLQRAPASKTLTLHTSAKVLILXLKRFSDVTGNKLAKNVQYPECLDMQPYMSQQNTGPLVYVLYAVLVHAGWSCHNGHYFSYVKAQEGQWYKMDDAEVTASSITSVLSQQAYVLFYIQKSEWERHSESVSRGREPRALGAEDTDRRATQGELKRDHPCLQAPELDEHLVERATQESTLDHWKFLQEQNKTKPEFNVRKVEGTLPPDVLVIHQSKYKCGMKNHHPEQQSSLLNLSSTTPTHQESMNTGTLASLRGRARRSKGKNKHSKRALLVCQ

SEQ ID NO: 12 - DNA, 735 bp, primate
  misc_feature (197): nucleotide may be A or C; resulting amino acid will reflect genetic code
atggctgtgccaagttggatcgtcaaacgcaggctactaccttggtccatcaaatttttggagggtatctcagatcacggcgtgaagtgctccgtgtgcaagagcgtctcggacacctacgacccctacttggacgtcgcgctggagatccggcaagctgcgaatattgtgcgtgctctggaactttttgtgaaagmagatgtcctgagtggagagaatgcctacatgtgtgctaaatgcaagaagaaggttccagccagcaagcgcttcaccatccacagaacatccaacgtcttaaccctttccctcaagcgctttgccaacttcagcggggggaagatcaccaaggatgtaggctatccggaattcctcaacatacgtccgtatatgtcccagaataatggtgatcctgtcatgtatggactctatgctgtcctggtgcactcgggctacagctgccatgccgggcactattactgctacgtgaaggcaagcaatggacagtggtaccagatgaatgattccttggtcccattccagcaacgtccaagttggtttctgaaaccagcaggcctaagtggcttgttctnatcggcgaatttccaggctctcaagaaaaattcccgaagggcctccattttccaggaacaggttccttcctccnnntcccgggcggcccgaattgtgaattccagattcattcccagnaggaacntcggcaatggggattatttt

SEQ ID NO: 13 - protein, 245 aa, primate
MAVPSWIVKRRLLPWSIKFLEGISDHGVKCSVCKSVSDTYDPYLDVALEIRQAANIVRALELFVKXDVLSGENAYMCAKCKKKVPASKRFTIHRTSNVLTLSLKRFANFSGGKITKDVGYPEFLNIRPYMSQNNGDPVMYGLYAVLVHSGYSCHAGHYYCYVKASNGQWYQMNDSLVPFQQRPSWFLKPAGLSGLFXSANFQALKKNSRRASIFQEQVPSSXSRAARIVNSRFIPXRNXGNGDYF

SEQ ID NO: 14 - DNA, 2244 bp, primate
  CDS (1)..(2244)
atgcagaaagcctgcctgaatggctgtgccaagttggatcgtcaaacgcaggctactaccttggtccatcaaatttttggagggtatctcagatcacgcgtgaagtgctccgtgtgcaagagcgtctcggacacctacgacccctacttggacgtcgcgctggagatccggcaagctgcgaatattgtgcgtgctctggaactttttgtgaaagcagatgtcctgagtggagagaatgcctacatgtgtgctaaatgcaagaagaaggttccagccagcaagcgcttcaccatccacagaacatccaacgtcttaaccctttccctcaagcgctttgccaacttcagcggggggaagatcaccaaggatgtaggctatccggaattcctcaacatacgtccgtatatgtcccagaataatggtgatcctgtcatgtatggactctatgctgtcctggtgcactcgggctacagctgccatgccgggcactattactgctacgtgaaggcaagcaatggacagtggtaccagatgaatgattccttggtccattccagcaacgtcaaggtggttctgaaccagcaggcctacgtgctgttctatctgcgaattccaggctctaagaaaagtcccgagggcctcatctccaggacaggctcctcctcccttcccggccgcccgagtgtgattccagatcactccaagaagaacatcggcaatgggattatttcctccccactgactggaaagcgacaagactctgggacgatgaagaagccgcacaccactgaagagattggtgtgcccatatccaggaatggctccaccctgggcctgaagtcccagaacggctgcattcctccaaagctgccctcggggtccccttcccccaaactctcccagacacccacacacatgccaaccatcctagacgaccctggaaagaaggtgaagaagccagctcctccacagcacttttcccccagaactgctcaggggctgcctgggaccagcaactcgaatagcagcagatctgggagccaaaggcagggctcctgggacagcagggatgttgtcctctctacctcacctaagctcctggctacagccactgccaacgggcatgggctgaaggggaacgacgagagcgctggcctcgacaggaggggctccagcagctccagcccagagcactcggccagcagcgactccaccaaggccccccagacccccaggagtggagcggcccatctctgcgattctcaggaaacgaactgttccaccgctggccactccaaaacgccgccaagtggagcagattctaagacggtgaagctgaagtcccctgtcctgagcaacaccaccactgagcctgcaagcaccatgtctcctccaccagccaaaaaactggccctttctgccaagaaggccagcaccctgtggagggcgaccggcaatgacctccgtccacctcccccctcaccatcctccgacctcacccaccccatgaaaacctctcaccccgtcgttgcctccacttggcccgtccatagagccagggctgtgtcacctgctccccaatcatccagccgcctgcaaccccccttcagcccccaccccacattgctgtccagtacccccaagcccccagggacgtcagaaccacggagctgctcctccatctcgacggcgctgcctcaggtcaacgaggaccttgtgtctcttccacaccagttgccagaggccagtgagcccccccagagcccctctgagaagaggaaaaagacctttgtgggagagccgcagaggctgggctcagagacgcgcctcccacagcacatcagggaggccactgcggctccccacgggaagaggaagaggaagaagaagaagcgcccggaggacacagctgccagcgccctgcaggaggggcagacacagagacagcctgggagccccatgtacaggagggagggccaggcacagctgcccgctgtcagacggcaggaagatggcacacagccacaggtgaatggccagcaggtgggatgtgttacggacggccaccacgcgagcagcaggaagcggaggaggaaaggagcagaaggtcttggtgaagaaggcggcctgcaccaggacccacttcggcacagctgctctcccatgggtgatggtgatccagaggccatggaagagtctccaaggaaaaagaaaaaaaaaaaaaactcgagggggggcccggta

SEQ ID NO: 15 - protein, 748 aa, primate
MQKACLNGCAKLDRQTQATTLVHQIFGGYLRSRVKCSVCKSVSDTYDPYLDVALEIRQAANIVRALELFVKADVLSGENAYMCAKCKKKVPASKRFTIHRTSNVLTLSLKRFANFSGGKITKDVGYPEFLNIRPYMSQNNGDPVMYGLYAVLVHSGYSCHAGHYYCYVKASNGQWYQMNDSLVHSSNVKVVLNQQAYVLFYLRIPGSKKSPEGLISRTGSSSLPGRPSVIPDHSKKNIGNGIISSPLTGKRQDSGTMKKPHTTEEIGVPISRNGSTLGLKSQNGCIPPKLPSGSPSPKLSQTPTHMPTILDDPGKKVKKPAPPQHFSPRTAQGLPGTSNSNSSRSGSQRQGSWDSRDVVLSTSPKLLATATANGHGLKGNDESAGLDRRGSSSSSPEHSASSDSTKAPQTPRSGAAHLCDSQETNCSTAGHSKTPPSGADSKTVKLKSPVLSNTTTEPASTMSPPPAKKLALSAKKASTLWRATGNDLRPPPPSPSSDLTHPMKTSHPVVASTWPVHRARAVSPAPQSSSRLQPPFSPHPTLLSSTPKPPGTSEPRSCSSISTALPQVNEDLVSLPHQLPEASEPPQSPSEKRKKTFVGEPQRLGSETRLPQHIREATAAPHGKRKRKKKKRPEDTAASALQEGQTQRQPGSPMYRREGQAQLPAVRRQEDGTQPQVNGQQVGCVTDGHHASSRKRRRKGAEGLGEEGGLHQDPLRHSCSPMGDGDPEAMEESPRKKKKKKNSRGGPV

SEQ ID NO: 16 - protein, 526 aa, primate
MVVALSFPEADPALSSPDAPELHQDEAQVVEELTVNGKHSLSWESPQGPGCGLQNTGNSCYLNAALQCLTHTPPLADYMLSQEHSQTCCSPEGCKLCAMEALVTQSLLHSHSGDVMKPSHILTSAFHKHQQEDAHEFLMFTLETMHESCLQVHRQSKPTSEDSSPIHDIFGGWWRSQIKCLLCQGTSDTYDRFLDIPLDISSAQSVKQALWDTEKSEELCGDNAYYCGKCRQKMPASKTLHVHIAPKVLMVVLNRFSAFTGNKLDRKVSYPEFLDLKPYLSEPTGGPLPYALYAVLVHDGATSHSGHYFCCVKAGHGKWYKMDDTKVTRCDVTSVLNENAYVLFYVQQANLKQVSIDMPEGRINEVLDPEYQLKKSRRKKHKKKSPFTEDLGEPCENRDKRAIKETSLGKGKVLQEVNHKKAGQKHGNTKLMPQKQNHQKAGQNLRNTEVELDLPADAIVIHQPRSTANWGRDSPDKENQPLHNADRLLTSQGPVNTWQLCRQEGRRRSKKGQNKNKQGQRLLLVC

SEQ ID NO: 17 - protein, 545 aa, primate
  SITE (414): Xaa from sequence problem
MVVSLSFPEADPALSSPGAQQLHQDEAQVVVELTANDKPSLSWECPQGPGCGLQNTGNSCYLNAALQCLTHTPPLADYMLSQEYSQTCCSPEGCKMCAMEAHVTQSLLHSHSGDVMKPSQILTSAFHKHQQEDAHEFLMFTLETMHESCLQVHRQSEPTSEDSSPIHDIFGGLWRSQIKCLHCQGTSDTYDRFLDVPLDISSAQSVNQALWDTEKSEELRGENAYYCGRCRQKMPASKTLHIHSAPKVLLLVLKRFSAFMGNKLDRKVSYPEFLDLKPYLSQPTGGPLPYALYAVLVHEGATCHSGHYFSYVKARHGAWYKMDDTKVTSCDVTSVLNENAYVLFYVQQTDLKQVSIDMPEGRVHEVLDPEYQLKKSRRKKHKKKSPCTEDAGEPCKNREKRATKETSLGEGKVXQEKNHKKAGQKHENTKLVPQEQNHQKLGQKHRINEILPQEQNHQKAGQSLRNTEGELDLPADAIVIHLLRSTENWGRDAPDKENQPWHNADRLLTSQDPVNTGQLCRQEGRRRSKKGKNKNKQGQRLLLVC

SEQ ID NO: 18 - DNA, 890 bp, primate
  CDS (15)..(500)
  misc_feature (53): nucleotide may be A or C; resulting amino acid will reflect genetic code
ggcacgagcccaccatgaagggtttcacagccactctcttcctctggactctmatttttcccagctgcagtggaggcggcggtgggaaagcctggcccacacacgtggtctgtagcgacagcskyttggaagtgctctaccagagttgcgatccattacaagattttggcttttctgttgaaaagtgttccaagcaattaaaatcaaatatcaacattagatttggaattattctgagagaggacatcaaagagctttttcttgacctagctctcatgtctcaaggctcatctgttttgaatttctcctatcccatctgtgaggcggctctgcccaagttttctttctgtggaagaaggaaaggagagcagatttactatgctgggcctgtcaataatcctgaatttactattcctcagggagaataccaggttttgctggaactgtacactgaaaaacggtccaccgtggcctgtgccaatgctactatcatgtgctcctgactgtgggcctgttagcaaaaantcacagccagctgcatctcgtngggaaccttccaagctcctctgactgaacctactgtgggaggagaagcagctgatgacagagagaggctctacaaagaagcgcccccaaagagtgcagctgctaattttagtcccaggaccagacatccccagactccacagatgtaatgaagtccccgaatgtatctgtttctaaggagcctcttggcagtccttaagcagtcttgagggtccatcctttttctctaattggtcgcctcccaccagactcacctgcttttcaactttttaggagtgcttcctcacangttaccaataataaagaaagctggccaccaaaaaaaaaaaaaaaaaaaaaaaaaa

SEQ ID NO: 19 - protein, 162 aa, primate
MKGFTATLFLWTXIFPSCSGGGGGKAWPTHVVCSDSXLEVLYQSCDPLQDFGFSVEKCSKQLKSNINIRFGIILREDIKELFLDLALMSQGSSVLNFSYPICEAALPKFSFCGRRKGEQIYYAGPVNNPEFTIPQGEYQVLLELYTEKRSTVACANATIMCS

SEQ ID NO: 20 - DNA, 486 bp, primate
  CDS (1)..(132)
cccctgttttcttccatatttactgaagctcagaagcagtattgggtctgcaactcatccgatgcaagtatttcatacacctactgtgataaaatgcaatacccaatttcaattaatgttaacccctgtatagaattgaaaggatccaaaggattattgcacattttctacattccaaggagagatttaaagcaattatatttcaatctctatataactgtcaacaccatgaatcttccaaagcgcaaagaagttatttgccgaggatctgatgacgattactctttttgcagagctctgaagggagagactgtgaatacaacaatatcattctccttcaagggaataaaattttctaagggaaaatacaaatgtgttgttgaagctatttctgggagcccagaagaaatgctcttttgcttggagtttgtcatcctacaccaacctaattcaaattagaataaattgagtatttaaaaaaaaaaa

SEQ ID NO: 21 - protein, 44 aa, primate
PLFSSIFTEAQKQYWVCNSSDASISYTYCDKMQYPISINVNPCI

SEQ ID NO: 22 - DNA, 483 bp, primate
  CDS (1)..(480)
atgttcccatttctgtttttttccaccctgttttcttccatatttactgaagctcagaagcagtattgggtctgcaactcatccgatgcaagtatttcatacacctactgtgataaaatgcaatacccaatttcaattaatgttaacccctgtatagaattgaaaggatccaaaggattattgcacattttctacattccaaggagagatttaaagcaattatatttcaatctctatataactgtcaacaccatgaatcttccaaagcgcaaagaagttatttgccgaggatctgatgacgattactctttttgcagagctctgaagggagagactgtgaatacaacaatatcattctccttcaagggaataaaattttctaagggaaaatacaaatgtgttgttgaagctatttctgggagcccagaagaaatgctcttttgcttggagtttgtcatcctacaccaacctaattcaaattag

SEQ ID NO: 23 - protein, 160 aa, primate
MFPFLFFSTLFSSIFTEAQKQYWVCNSSDASISYTYCDKMQYPISINVNPCIELKGSKGLLHIFYIPRRDLKQLYFNLYITVNTMNLPKRKEVICRGSDDDYSFCRALKGETVNTTISFSFKGIKFSKGKYKCVVEAISGSPEEMLFCLEFVILHQPNSN

SEQ ID NO: 24 - DNA, 498 bp, rodent
  CDS (53)..(394)
gtcgagtccgatggtcttcctggcgagtttaaagtatcggagatattaaatcatgttgccatttattctcttttcgacgctgctttctcccatattgactgaatctgagaagcaacagtggttctgcaactcctccgatgcaattatttcctacagttattgtgatcacttgaaattccctatttcaattagttctgaaccctgcataagactgaggggaaccaatggatttgtgcatgttgagttcattccaagaggaaacttaaaatatttatatttcaacctattcatcagtgtcaactccatagagttgccgaagcgtaaggaagttctgtgccatggacatgatgatgactattctttttgcagagctctgaaaggaggatatgctatttagaaaatatgagactgtgaatacatcaataccattctctttcgagggaatactatttcctaagggccattacagatgtgttgcagaagctattgctggggata

SEQ ID NO: 25 - protein, 114 aa, rodent
MLPFILFSTLLSPILTESEKQQWFCNSSDAIISYSYCDHLKFPISISSEPCIRLRGTNGFVHVEFIPRGNLKYLYFNLFISVNSIELPKRKEVLCHGHDDDYSFCRALKGGYAI

SEQ ID NO: 26 - protein, 141 aa, primate
MLPFILFSTLLSPILTESEKQQWFCNSSDAIISYSYCDHLKFPISISSEPCIRLRGTNGFVHVEFIPRGNLKYLYFNLFISVNSIELPKRKEVLCHGHDDDYSFCRALKGETVNTSIPFSFEGILFPKGHYRCVAEAIAGD

SEQ ID NO: 27 - protein, 162 aa, rodent
MNGVAAALLVWILTSPSSSDHGSENGWPKHTACNSGGLEVVYQSCDPLQDFGLSIDQCSKQIQSNLNIRFGIILRQDIRKLFLDITLMAKGSSILNYSYPLCEEDQPKFSFCGRRKGEQIYYAGPVNNPGLDVPQGEYQLLLELYNENRATVACANATVTSS

SEQ ID NO: 28 - DNA, 1158 bp, aves
  CDS (11)..(490)
cggtgcaaccatgaagacattgaatgttctcgctctcgtcttagtcctgctttgcatcaatgccagcacagagtggcctacacacacagtctgcaaggaggaaaacttggagatatattacaaaagctgtgatccccagcaagactttgctttcagcattgaccgttgttcagatgtcacaacccacacctttgacatcagagctgcaatggtcctaagacaaagcatcaaggaactgtatgccaaggttgatctgatcataaatgggaagactgtcttaagctactcagagacactctgtggaccaggcctttctaagctaattttctgtggaaagaagaaaggagaacatctctactatgagggaccaatcacactgggaatcaaagaaatcccacagcgagattacactatcacagcaaggctgactaacgaagatcgcgccactgttgcttgtgctgattttaccgtgaaaaattatttagattattaagcaaaacaacgcactcggtccgactcccttaaaactacagattcctaaaactattcaagcccagtgagctgcttgcatgcttcagtgattctgaaggaaagatctcccgcacggtggttctgatgctgttcctcttcgtaattcaacttttttggagaagtcactaggccctaccctctagtggtaattttatctccaaatgcactctgtagcccacttttcgcttttaatatatacagctgcaaatagaaagtatttgataccaacattctcatctcaggatgaaaatagtacaaagcagaagaggcgagagccaaaacagatttttgcagtaagctatggaggtatccatttctaacacaagctaaagaagattgtcatatgtattatgcagttatagcactcaacattttcagtttttcacaaggcctgtttggagcctccattggtataaattttgttgtaaccacagaacaaagaccaaataggatgaacatggctccatgttcagtcactctattcatatcatttaagttttcatgattcttcttgtatatttttttttattctttaatgtttacagtgatgtgagaatccttttgtttaagctacatgctgttcccgcttgtcaataaatctgcaagaaaaaaaaaaaaaaaaaaaaa

SEQ ID NO: 29 - protein, 160 aa, aves
MKTLNVLALVLVLLCINASTEWPTHTVCKEENLEIYYKSCDPQQDFAFSIDRCSDVTTHTFDIRAAMVLRQSIKELYAKVDLIINGKTVLSYSETLCGPGLSKLIFCGKKKGEHLYYEGPITLGIKEIPQRDYTITARLTNEDRATVACADFTVKNYLDY

(19) United States
(12) Patent Application Publication
(10) Pub. No.: US 2003/0028005 A1
(43) Pub. Date: Feb. 6, 2003

(54) MAMMALIAN PROTEINS; RELATED REAGENTS AND METHODS

(76) Inventor: J. FERNANDO BAZAN, MENLO PARK, CA (US)

Correspondence Address: DNAX RESEARCH INSTITUTE, LEGAL DEPARTMENT, 901 CALIFORNIA AVENUE, PALO ALTO, CA, 94304, US

(21) Appl. No.: 09/372,348

(22) Filed: Aug. 11, 1999

Related U.S. Application Data
(60) Provisional application No. 60/096,328, filed on Aug. 12, 1998.

(51) Int. Cl. A01N 37/18; A61K 38/00; G01N 33/53; C07H 21/04; C07K 5/00; C07K 7/00; C07K 16/00; C07K 17/00; C12N 5/00; C12N 5/02; C07K 1/00; C07K 14/00

(52) U.S. Cl. 536/23.5; 530/324; 514/2; 530/387.1; 435/325; 435/7.1

(57) ABSTRACT

Novel CC chemokine, de-ubiquitination, and cell surface proteins from mammals, reagents related thereto including purified proteins, specific antibodies and nucleic acids encoding these antigen are provided. Also provided are methods of making and using said reagents and diagnostic kits.

The present filing is a conversion of Provisional U.S. Patent Application U.S. Ser. No. 60/096,328, filed Aug. 12, 1998, which is incorporated herein by reference, to a U.S. Utility Patent Application.

### FIELD OF THE INVENTION

The present invention contemplates compositions related to proteins which function in controlling development, differentiation, trafficking, and physiology of mammalian cells, e.g., cells of a mammalian immune system. In particular, it provides proteins which regulate or evidence development, differentiation, and function of various cell types, including hematopoietic cells.

### BACKGROUND OF THE INVENTION

The circulating component of the mammalian circulatory system comprises various cell types, including red and white blood cells of the erythroid and myeloid cell lineages. See, e.g., Rapaport (1987) Introduction to Hematology (2d ed.) Lippincott, Philadelphia, Pa.; Jandl (1987) Blood: Textbook of Hematology, Little, Brown and Co., Boston, Mass.; and Paul (ed. 1993) Fundamental Immunology (3d ed.) Raven Press, N.Y.

For some time, it has been known that the mammalian immune response is based on a series of complex cellular interactions, called the “immune network.” Recent research has provided new insights into the inner workings of this network. While it remains clear that much of the response does, in fact, revolve around the network-like interactions of lymphocytes, macrophages, granulocytes, and other cells, immunologists now generally hold the opinion that soluble proteins, known as lymphokines, cytokines, or monokines, play a critical role in controlling these cellular interactions. Thus, there is considerable interest in the isolation, characterization, and mechanisms of action of cell modulatory factors, an understanding of which should lead to significant advancements in the diagnosis and therapy of numerous medical abnormalities, e.g., immune system and other disorders.

Lymphokines apparently mediate cellular activities in a variety of ways. They have been shown to support the proliferation, growth, and differentiation of the pluripotential hematopoietic stem cells into vast numbers of progenitors comprising diverse cellular lineages making up a complex immune system. These interactions between the cellular components are necessary for a healthy immune response. These different cellular lineages often respond in a different manner when lymphokines are administered in conjunction with other agents.

The chemokines are a large and diverse superfamily of proteins. The superfamily is subdivided into two classical branches, based upon whether the first two cysteines in the chemokine motif are adjacent (termed the “C—C” branch), or spaced by an intervening residue (“C—X—C”). A more recently identified branch of chemokines lacks two cysteines in the corresponding motif, and is represented by the chemokines known as lymphotactins. Another recently identified branch has three intervening residues between the two cysteines, e.g., CX3C chemokines. See, e.g., Schall and Bacon (1994) Current Opinion in Immunology 6:865-873; and Bacon and Schall (1996) Int. Arch. Allergy & Immunol. 109:97-109.

Because the physiology mediated by these soluble molecules is so important, the discovery of novel chemokines will be important, both in diagnostic and therapeutic contexts.

In addition, while the general importance of the regulation of protein synthesis is universally accepted, the general importance of protein degradation has not been fully appreciated. One mechanism of protein degradation is via ubiquitination signals and degradation pathways. Ubiquitin (Ub) is a highly conserved 76 amino acid polypeptide that plays an important role in the regulation of protein degradation, cell-cycle progression, gene transcription and signal transduction. The ubiquitination pathway is fine tuned and controlled, in part, by deubiquitination enzymes, which remove ubiquitin from proteins. Misregulation of the ubiquitination pathway may contribute problems in the protein quantity regulation, which may be associated, e.g., with malignant transformation, and oncogenesis through oncogenic counterparts of normally processed ubiquitinated proteins. Other clinical problems will often result from excessive or insufficient protein levels. Therefore, understanding the ubiquitination roles, e.g., in immune function, will increase our understanding of cell biology, which should have relevance, e.g., to malignant transformation.

Furthermore, growth of normal resting B cells (also referred to as “B lymphocytes”) involves two distinct steps. First, the resting cells are activated to pass from the Go to G₁ phase of the cell cycle. See, e.g., Alberts, et al. (eds. 1989) Molecular Biology of the Cell Garland Publ., NY; and Darnell, et al. (1990) Molecular Cell Biology Freeman, NY. Next, the activated cells are induced to proliferate. See, e.g., Paul, ed. (1989) Fundamental Immunology, 2nd ed., Raven Press, NY; and the third edition. Several factors have been identified that induce growth of B cells, including interleukin-1 (IL-1), IL-2, IL-4, IL-10, and IL-13. In addition, antibodies against certain B cell surface molecules have been demonstrated to promote B cell proliferation. T cells (also referred to as “T lymphocytes”) are also induced to proliferate by certain factors, which include phytohemagglutinin, anti-T cell receptor monoclonal antibodies, anti-CD3 monoclonal antibodies, and other agents.

B7 (CD80) and B70 (CD86) are the second “group” of molecules which strongly mediate B and T cell interaction. These molecules, on B cells, interact with their ligands CD28 and CTLA-4 on T cells. These interactions are major co-stimulatory signals for activation of both B and T cells.

During the last 15 years, it has become apparent that B7 (CD80) and B70 (CD86) play fundamental functions in T cell and B cell activation Numerous in vitro and in vivo experiments have demonstrated that these two pairs of molecules represent important targets for immunosuppression. See, e.g., Banchereau, et al. (1994) Ann. Rev. Immunol. 12:881-922; van Kooten, et al. (1996) Adv. Immunol. 61:1-77; Linsley and Ledbetter (1993) Ann. Rev. Immunol. 11:191-212.

In 1995, another molecule called RP105 was cloned from mouse splenic cells. See Miyake, et al (1995) J. Immunol. 154:3333-3340. Monoclonal antibodies against RP105 also induce strong proliferation of mouse B cells and protects mouse B cells from irradiation-induced apoptosis in a similar fashion to anti-CD40 antibody or CD40-ligand. See Miyake, et al. (1994) J. Exp. Med. 180:1217-1224.

The RP105 molecule and its ligand MD1 may be an additional pair of molecules that play key roles in the activation of T cells and B cells. See Miyake, et al. (1998) J. Immunol. 161:1349-1353; and Chan, et al., (1998) J. Exp. Med. 188:93-101 However, the human sequence of MD1, has remained undetermined. The present invention provides this and also provides a previously undescribed second human homolog of mouse MD1, (i.e., MD2).

Many factors have been identified which influence the differentiation process of precursor cells, or regulate the physiology or migration properties of specific cell types. These observations indicate that other factors exist whose functions in immune function were heretofore unrecognized. These factors provide for biological activities whose spectra of effects may be distinct from known differentiation or activation factors. The absence of knowledge about the structural, biological, and physiological properties of the regulatory factors which regulate cell physiology in vivo prevents the modulation of the effects of such factors. Thus, medical conditions where regulation of the development or physiology of relevant cells is required remains unmanageable.

Thus, significant therapeutic needs exist in the areas of cytokine regulation of physiology, protein degradation, and B cell signaling. The present invention provides important insights and developments in these areas.

### SUMMARY OF THE INVENTION

The present invention reveals the existence of various novel genes, and proteins derived therefrom.

In one embodiment, the invention provides a previously unknown chemokine-motif containing molecule which is designated human HCC5. In another embodiment, the invention provides genes exhibiting similarity to deubiquinating enzymes. In yet another embodiment, the invention provides genes exhibiting similarity to ligands for receptor proteins on B cells, e.g., the RP105 surface protein.

The present invention provides a composition of matter selected from: a substantially pure or recombinant HCC5 protein or peptide exhibiting identity over a length of at least 12 amino acids to SEQ ID NO: 2; an isolated natural sequence HCC5 of SEQ ID NO: 2; a fusion protein comprising HCC5 sequence; a substantially pure or recombinant Dub11 protein or peptide exhibiting identity over a length of at least about 12 amino acids to SEQ ID NO: 9 or 11; an isolated natural sequence Dub11 of SEQ ID NO: 9 or 11; and a fusion protein comprising Dub11 sequence; a substantially pure or recombinant Dub12 protein or peptide exhibiting identity over a length of at least about 12 amino acids to SEQ ID NO: 13 or 15; an isolated natural sequence Dub12 of SEQ ID NO: 13 or 15; and a fusion protein comprising Dub12 sequence; a substantially pure or recombinant primate MD1 protein or peptide exhibiting identity over a length of at least about 12 amino acids to SEQ ID NO: 19; an isolated natural sequence primate MD1 of SEQ ID NO: 19; and a fusion protein comprising primate MD1 sequence; a substantially pure or recombinant primate MD2 protein or peptide exhibiting identity over a length of at least about 12 amino acids to SEQ ID NO: 21 or 23; an isolated natural sequence primate MD2 of SEQ ID NO: 21 or 23; and a fusion protein comprising primate MD2 sequence; a substantially pure or recombinant rodent MD2 protein or peptide exhibiting identity over a length of at least about 12 amino acids to SEQ ID NO: 25 or 26; an isolated natural sequence rodent MD2 of SEQ ID NO: 25 or 26; and a fusion protein comprising rodent MD2 sequence.

In preferred embodiments, the substantially pure or isolated protein is: A) an HCC5, wherein: the length of identity is at least 17 amino acids; the length is at least 21 amino acids; or the length is at least 27 amino acids; or B) a Dub11, wherein: the length of identity is at least 17 amino acids; the length is at least 21 amino acids; or the length is at least 27 amino acids; or C) a Dub12, wherein: the length of identity is at least 17 amino acids; the length is at least 21 amino acids; or the length is at least 27 amino acids; or D) a primate MD1, wherein: the length of identity is at least 17 amino acids; the length is at least 21 amino acids; or the length is at least 27 amino acids; or E) a primate MD2, wherein: the length of identity is at least 17 amino acids; the length is at least 21 amino acids; or the length is at least 27 amino acids; or F) a rodent MD2, wherein: the length of identity is at least 17 amino acids; the length is at least 21 amino acids; or the length is at least 27 amino acids.

Other preferred embodiments include those wherein the: A) HCC5: polypeptide: is from a primate, including a human; comprises at least one polypeptide segment of SEQ ID NO: 2; exhibits a plurality of portions exhibiting the identity; is a natural allelic variant of HCC5; has a length at least about 30 amino acids; exhibits at least two non-overlapping epitopes which are specific for a primate HCC5; exhibits a sequence identity over a length of at least 35 amino acids to an HCC5; exhibits at least two non-overlapping epitopes which are specific for a HCC5; is glycosylated; is a synthetic polypeptide; is attached to a solid substrate; is conjugated to another chemical moiety; is a 5-fold or less substitution from natural sequence; or is a deletion or insertion variant from a natural sequence; or B) the Dub11: protein comprises a mature sequence of Table 2; or polypeptide: is from a primate, including a human; comprises at least one polypeptide segment of SEQ ID NO: 4; exhibits a plurality of portions exhibiting the identity; is a natural allelic variant of Dub11; has a length at least about 30 amino acids; exhibits at least two non-overlapping epitopes which are specific for a primate Dub11; exhibits a sequence identity over a length of at least about 35 amino acids to a Dub11; exhibits at least two non-overlapping epitopes which are specific for a Dub11; is glycosylated; is a synthetic polypeptide; is attached to a solid substrate; is conjugated to another chemical moiety; is a 5-fold or less substitution from natural sequence; or is a deletion or insertion variant from a natural sequence; or C) the Dub12: comprises a mature sequence of Table 2; or protein or peptide: is from a primate, including a human; comprises at least one polypeptide segment of SEQ ID NO: 4; exhibits a plurality of portions exhibiting the identity; is a natural allelic variant of Dub12; has a length at least about 30 amino acids; exhibits at least two non-overlapping epitopes which are specific for a primate Dub12; exhibits a sequence identity over a length of at least about 35 amino acids to a Dub12; exhibits at least two non-overlapping epitopes which are specific for a Dub12; is glycosylated; is a synthetic polypeptide; is attached to a solid substrate; is conjugated to another chemical moiety; is a 5-fold or less substitution from natural sequence; or is a deletion or insertion variant from a natural sequence; or D) the primate MD1: comprises a mature sequence of Table 3; or polypeptide: is from a primate, including a human; comprises at least one polypeptide segment of SEQ ID NO: 19; exhibits a plurality of portions exhibiting the identity; is a natural allelic variant of primate MD1; has a length at least about 30 amino acids; exhibits at least two non-overlapping epitopes which are specific for a primate MD1; exhibits a sequence identity over a length of at least about 35 amino acids to a primate MD1; exhibits at least two non-overlapping epitopes which are specific for a primate MD1; is glycosylated; is a synthetic polypeptide; is attached to a solid substrate; is conjugated to another chemical moiety; is a 5-fold or less substitution from natural sequence; or is a deletion or insertion variant from a natural sequence; or E) the primate MD2: comprises a mature sequence of Table 3; or polypeptide: is from a primate, including a human; comprises at least one polypeptide segment of SEQ ID NO: 21 or 23; exhibits a plurality of portions exhibiting the identity; is a natural allelic variant of primate MD2; has a length at least about 30 amino acids; exhibits at least two non-overlapping epitopes which are specific for a primate MD2; exhibits a sequence identity over a length of at least about 35 amino acids to a primate MD2; exhibits at least two non-overlapping epitopes which are specific for a primate MD2; is glycosylated; is a synthetic polypeptide; is attached to a solid substrate; is conjugated to another chemical moiety; is a 5-fold or less substitution from natural sequence; or is a deletion or insertion variant from a natural sequence; or F) the rodent MD2: comprises a mature sequence of Table 3; or polypeptide: is from a rodent, including a mouse; comprises at least one polypeptide segment of SEQ ID NO: 25 or 26; exhibits a plurality of portions exhibiting the identity; is a natural allelic variant of rodent MD2; has a length at least about 30 amino acids; exhibits at least two non-overlapping epitopes which are specific for a rodent MD2; exhibits a sequence identity over a length of at least about 35 amino acids to a rodent MD2; exhibits at least two non-overlapping epitopes which are specific for a rodent MD2; is glycosylated; is a synthetic polypeptide; is attached to a solid substrate; is conjugated to another chemical moiety; is a 5-fold or less substitution from natural sequence; or is a deletion or insertion variant from a natural sequence.

Additional compositions are provided, e.g., A) a sterile HCC5 polypeptide; or B) the HCC5 polypeptide and: a carrier, wherein the carrier is: an aqueous compound, including water, saline, and/or buffer; and/or formulated for oral, rectal, nasal, topical, or parenteral administration; or another chemokine, including one selected from the group of HCC1, HCC2, HCC3, and HCC4; or an antibody antagonist for a chemokine, including one selected from the group of HCC1, HCC2, HCC3, and HCC4; or C) a sterile Dub11 polypeptide; or D) the Dub11 polypeptide and: a carrier, wherein the carrier is: an aqueous compound, including water, saline, and/or buffer; and/or formulated for oral, rectal, nasal, topical, or parenteral administration; or E) a sterile Dub12 polypeptide; or F) the Dub12 polypeptide and: a carrier, wherein the carrier is: an aqueous compound, including water, saline, and/or buffer; and/or formulated for oral, rectal, nasal, topical, or parenteral administration; or G) a sterile MD1 polypeptide; or H) the MD1 polypeptide and: 1) a carrier, wherein the carrier is: an aqueous compound, including water, saline, and/or buffer; and/or formulated for oral, rectal, nasal, topical, or parenteral administration; or I) a sterile MD2 polypeptide; the MD2 polypeptide and: a carrier, wherein the carrier is: an aqueous compound, including water, saline, and/or buffer; and/or formulated for oral, rectal, nasal, topical, or parenteral administration.

Fusion protein embodiments include those comprising: mature protein sequence of Table 1; mature protein sequence of Table 2; mature protein sequence of Table 3; a detection or purification tag, including a FLAG, His6, or Ig sequence; or sequence of another chemokine protein with the protein described.

The invention further provides a kit comprising such a protein or polypeptide, and: a compartment comprising the protein or polypeptide; and/or instructions for use or disposal of reagents in the kit.

Antibody and binding compound embodiments include a binding compound comprising an antigen binding portion from an antibody, which specifically binds to a natural: A) HCC5 polypeptide, wherein: the polypeptide is a primate polypeptide; the binding compound is an Fv, Fab, or Fab2 fragment; the binding compound is conjugated to another chemical moiety; or the antibody: is raised against a peptide sequence of a mature polypeptide sequence of Table 1; is raised against a mature HCC5; is raised to a purified HCC5; is immunoselected; is a polyclonal antibody; binds to a denatured HCC5; exhibits a Kd to antigen of at least 30 μM; is attached to a solid substrate, including a bead or plastic membrane; is in a sterile composition; or is detectably labeled, including a radioactive or fluorescent label; or B) Dub11 polypeptide, wherein: the polypeptide is a primate polypeptide; the binding compound is an Fv, Fab, or Fab2 fragment; the binding compound is conjugated to another chemical moiety; or the antibody: is raised against a peptide sequence of a mature polypeptide sequence of Table 2; is raised against a mature Dub11; is raised to a purified Dub11; is immunoselected; is a polyclonal antibody; binds to a denatured Dub11; exhibits a Kd to antigen of at least 30 μM; is attached to a solid substrate, including a bead or plastic membrane; is in a sterile composition; or is detectably labeled, including a radioactive or fluorescent label; or C) Dub12 polypeptide, wherein: the polypeptide is a primate polypeptide; the binding compound is an Fv, Fab, or Fab2 fragment; the binding compound is conjugated to another chemical moiety; or the antibody: is raised against a peptide sequence of a mature polypeptide sequence of Table 2; is raised against a mature Dub12; is raised to a purified Dub12; is immunoselected; is a polyclonal antibody; binds to a denatured Dub12; exhibits a Kd to antigen of at least 30 μM; is attached to a solid substrate, including a bead or plastic membrane; is in a sterile composition; or is detectably labeled, including a radioactive or fluorescent label; or E) a primate MD1 polypeptide, wherein: the polypeptide is a human polypeptide; the binding compound is an Fv, Fab, or Fab2 fragment; the binding compound is conjugated to another chemical moiety; or the antibody: is raised against a peptide sequence of a mature polypeptide sequence of Table 3; is raised against a mature MD1; is raised to a purified MD1; is immunoselected; is a polyclonal antibody; binds to a denatured MD1; exhibits a Kd to antigen of at least 30 μM; is attached to a solid substrate, including a bead or plastic membrane; is in a sterile composition; or is detectably labeled, including a radioactive or fluorescent label; or F) a primate MD2 polypeptide, wherein: the polypeptide is a human polypeptide; the binding compound is an Fv, Fab, or Fab2 fragment; the binding compound is conjugated to another chemical moiety; or the antibody: is raised against a peptide sequence of a mature polypeptide sequence of Table 3; is raised against a mature MD2; is raised to a purified MD2; is immunoselected; is a polyclonal antibody; binds to a denatured MD2; exhibits a Kd to antigen of at least 30 μM; is attached to a solid substrate, including a bead or plastic membrane; is in a sterile composition; or is detectably labeled, including a radioactive or fluorescent label; or G) a rodent MD2 polypeptide, wherein: the polypeptide is a primate polypeptide; the binding compound is an Fv, Fab, or Fab2 fragment; the binding compound is conjugated to another chemical moiety; or the antibody: is raised against a peptide sequence of a mature polypeptide sequence of Table 3; is raised against a mature rodent MD2; is raised to a purified rodent MD2; is immunoselected; is a polyclonal antibody; binds to a denatured rodent MD2; exhibits a Kd to antigen of at least 30 μM; is attached to a solid substrate, including a bead or plastic membrane; is in a sterile composition; or is detectably labeled, including a radioactive or fluorescent label.

Preferred kits include those comprising such a binding compound, and: a compartment comprising the binding compound; and/or instructions for use or disposal of reagents in the kit.

The invention provides methods of producing an antigen:antibody complex, comprising contacting such an antibody with: A) a primate HCC5 polypeptide or B) a primate Dub11 polypeptide; or C) a primate Dub12 polypeptide; or D) a primate MD1 polypeptide; or E) a primate MD2 polypeptide; or F) a rodent MD2 polypeptide or peptide; thereby allowing the complex to form.

Related compositions include those comprising: a sterile binding compound; or the binding compound, as described, and: a carrier, wherein the carrier is: an aqueous compound, including water, saline, and/or buffer; and/or formulated for oral, rectal, nasal, topical, or parenteral administration; or an antibody antagonist for another chemokine, including one selected from the group of HCC1, HCC2, HCC3, and HCC4.

Nucleic acid compositions are provided, e.g., an isolated or recombinant nucleic acid encoding a polypeptide or fusion protein, wherein: A) the HCC5: polypeptide is from a primate, including a human; or nucleic acid: encodes an antigenic peptide sequence of Table 1; encodes a plurality of antigenic peptide sequences of Table 1; exhibits identity over at least 25 nucleotides to a natural cDNA encoding the segment; is an expression vector; further comprises an origin of replication; is from a natural source; comprises a detectable label; comprises synthetic nucleotide sequence; is less than 6 kb, preferably less than 3 kb; is from a primate, including a human; comprises a natural full length coding sequence; is a hybridization probe for a gene encoding the HCC5 polypeptide; is a PCR primer, PCR product, or mutagenesis primer; or further encodes another chemokine, including one selected from the group of HCC1, HCC2, HCC3, and HCC4; or B) the Dub11: polypeptide is from a primate, including a human; or nucleic acid: encodes an antigenic peptide sequence of Table 2; encodes a plurality of antigenic peptide sequences of Table 2; exhibits identity over at least 25 nucleotides to a natural cDNA encoding the segment; is an expression vector; further comprises an origin of replication; is from a natural source; comprises a detectable label; comprises synthetic nucleotide sequence; is less than 6 kb, preferably less than 3 kb; is from a primate, including a human; comprises a natural full length coding sequence; is a hybridization probe for a gene encoding the Dub11 polypeptide; or is a PCR primer, PCR product, mutagenesis primer; or C) the Dub12: polypeptide is from a primate, including a human; or nucleic acid: encodes an antigenic peptide sequence of Table 2; encodes a plurality of antigenic peptide sequences of Table 2; exhibits identity over at least 25 nucleotides to a natural cDNA encoding the segment; is an expression vector; further comprises an origin of replication; is from a natural source; comprises a detectable label; comprises synthetic nucleotide sequence; is less than 6 kb, preferably less than 3 kb; is from a primate, including a human; comprises a natural full length coding sequence; is a hybridization probe for a gene encoding the Dub11 polypeptide; or is a PCR primer, PCR product, or mutagenesis primer; or D) the primate MD1: polypeptide is from a human, including a human; or nucleic acid: encodes an antigenic peptide sequence of Table 3; encodes a plurality of antigenic peptide sequences of Table 3; exhibits identity over at least 25 nucleotides to a natural cDNA encoding the segment; is an expression vector; further comprises an origin of replication; is from a natural source; comprises a detectable label; comprises synthetic nucleotide sequence; is less than 6 kb, preferably less than 3 kb; is from a primate, including a human; comprises a natural full length coding sequence; is a hybridization probe for a gene encoding the Dub11 polypeptide; or is a PCR primer, PCR product, or mutagenesis primer; or E) the primate MD2: polypeptide is from a human; or nucleic acid: encodes an antigenic peptide sequence of Table 3; encodes a plurality of antigenic peptide sequences of Table 3; exhibits identity over at least 25 nucleotides to a natural cDNA encoding the segment; is an expression vector; further comprises an origin of replication; is from a natural source; comprises a detectable label; comprises synthetic nucleotide sequence; is less than 6 kb, preferably less than 3 kb; is from a human; comprises a natural full length coding sequence; is a hybridization probe for a gene encoding the primate MD2 polypeptide; or is a PCR primer, PCR product, or mutagenesis primer; or F) the rodent MD2: polypeptide is from a mouse; or nucleic acid: encodes an antigenic peptide sequence of Table 3; encodes a plurality of antigenic peptide sequences of Table 3; exhibits identity over at least 25 nucleotides to a natural cDNA encoding the segment; is an expression vector; further comprises an origin of replication; is from a natural source; comprises a detectable label; comprises synthetic nucleotide sequence; is less than 6 kb, preferably less than 3 kb; is from a mouse; comprises a natural full length coding sequence; is a hybridization probe for a gene encoding the rodent MD2 polypeptide; or is a PCR primer, PCR product, or mutagenesis primer.

The invention further provides a cell or tissue comprising such a recombinant nucleic acid, including wherein the cell is: a prokaryotic cell; a eukaryotic cell; a bacterial cell; a yeast cell; an insect cell; a mammalian cell; a mouse cell; a primate cell; or a human cell.

Other embodiments include a kit comprising such a nucleic acid, and: a compartment comprising the nucleic acid; a compartment comprising a nucleic acid encoding another chemokine, including HCC1, HCC2, HCC3, and HCC4; and/or instructions for use or disposal of reagents in the kit.

Other preferred nucleic acids include those which hybridize under wash conditions of 45° C. and less than 2M salt: to SEQ ID NO: 1; to SEQ ID NO: 8 or 10; to SEQ ID NO: 12 or 14; to SEQ ID NO: 18; to SEQ ID NO: 20 or 22; or to SEQ ID NO: 24; or wherein the wash conditions are at 55° C. and/or 500 mM salt; 60° C. and/or 200 mM salt; or wherein the wash conditions are at 65° C. and/or 150 mM salt.

Additional methods are provided, e.g., modulating physiology or development of a cell or tissue culture cells comprising exposing the cell to an agonist or antagonist of HCC5, primate MD1, primate MD2, or rodent MD2. It also provides a method of detecting specific binding to a compound, comprising: contacting the compound to a composition selected from: an antigen binding site which specifically binds to a HCC5 chemokine; an antigen binding site which specifically binds to a HCC5 chemokine; an expression vector encoding a HCC5 chemokine or fragment thereof; an expression vector encoding a HCC5 chemokine or fragment thereof; a substantially pure polypeptide which is specifically recognized by the described antigen binding site; a substantially pure polypeptide which is specifically recognized by the described antigen binding site; a substantially pure HCC5 chemokine or peptide, or a fusion protein comprising a 30 amino acid sequence portion of HCC5 chemokine sequence; a substantially pure HCC5 chemokine or peptide, or a fusion protein comprising a 30 amino acid sequence portion of HCC5 chemokine sequence; and detecting binding of the compound to the composition.

### DETAILED DESCRIPTION

Outline

I. General

II. Definitions

III. Nucleic Acids

IV. Making Proteins

V. Antibodies

a. antibody production

b. immunoassays

VI. Purified proteins

VII. Physical Variants

VIII. Binding Agent:Antigen Complexes

IX. Functional Variants

X. Uses

XI. Kits

XII. Ligand Receptor Isolation

I. General

The present invention provides mammalian, e.g., primate, DNA sequences encoding proteins which exhibit structural properties or motifs characteristic of various proteins.

In one embodiment, the invention provides a chemokine. For a review of the chemokine family, see, e.g., Lodi, et al. (1994) Science 263:1762-1767; Gronenborn and Clore (1991) Protein Engineering 4:263-269; Miller and Kranger (1992) Proc. Nat'l Acad. Sci. USA 89:2950-2954; Matsushima and Oppenheim (1989) Cytokine 1:2-13; Stoeckle and Baker (1990) New Biol. 2:313-323; Oppenheim, et al. (1991) Ann. Rev. Immunol. 9:617-648; Schall (1991) Cytokine 3:165-183; and Thomson (ed. 1994) The Cytokine Handbook 2d ed. Academic Press, NY.

The new chemokine described herein is designated HCC5 which is a CC chemokine. See Table 1. The descriptions below are directed, for exemplary purposes, to the human HCC5 natural allele described, but are likewise applicable to allelic and/or polymorphic variants, e.g., from other individuals, as well as splicing variants, e.g., natural forms. Based on sequence analysis of the chemokine protein sequences described below, it is apparent that HCC5 belongs to the CC chemokine family. See, e.g., human stem cell mobilizing chemokine (CKbeta-1): Kreider, et al. (1997) Patent WO 9715594 (SEQ ID NO: 3) is GenBank Accession number 97P-W17659; human macrophage inflammatory protein-1-gamma (MIP-1): Adams, et al. (1995) Patent WO 9517092 (SEQ ID NO: 4) is GenBank Accession number 95P-R76128; human MIP-4: a chemoattractant for leukocytes; Adams, et al. (1997) Patent WO 9634891 (SEQ ID NO: 5) is GenBank Accession number 96P-WO7203; human pituitary expressed chemokine (PGEC): Bandman, et al. Patent WO 9616979 (SEQ ID NO: 6) is GenBank Accession number 96P-R95691; and human chemokine HCC1: Forsmann, et al. (1998) Patent WO 9741230 (SEQ ID NO: 7) is GenBank Accession number 97P-W38171.

These genes will allow isolation of other mammalian genes encoding proteins related to this, further extending the family beyond the specific embodiment described. The procedure is broadly set forth below.

The present invention also provides mammalian, e.g., primate, DNA sequences encoding proteins which exhibit structural properties of a deubiquitinating protein. These proteins are designated deubiquitinating protein 11 (Dub11) and deubiqutinating protein 12 (Dub12). See Table 2. For a review of the superfamily of deubiquitinating enzymes see, e.g., Hochstrasser (1995) Curr. Opin. Cell Biol. 7:215-223; Wilkinson, et al. (1995) Biochemistry 34:14535-14546; Baker, et al. (1992) J. Biol. Chem. 267:23364-23375; and Papa and Hochstrasser (1993) Nature 366:313-319. However, the deubiquitinating enzymes have also been reported to have additional functions besides deubiquitination. See, e.g., Hochstrasser (1996) Cell 84:813-815; Hicke and Riezman (1996) Cell 84:277-287; and Chen, et al. (1996) Cell 84:853-862.

The descriptions below are directed, for exemplary purposes, to the human Dub11 and human Dub12 natural alleles described, but are likewise applicable to allelic and/or polymorphic variants, e.g., from other individuals, as well as splicing variants, e.g., natural forms, and species variants from other primates or other species. These genes will allow isolation of other primate genes encoding proteins related to this, further extending the family beyond the specific embodiments described.

The present invention also provides mammalian, e.g., primate, and rodent e.g., mouse DNA sequences encoding proteins which exhibit structural properties of ligands for proteins exhibiting a leucine-rich protein motif (LRR) that is important, e.g., in immune function. These proteins are designated herein human MD1, human MD2, and murine MD2. See Table 3. The human MD1 is a primate homologue of the previously described rodent MD1, see, e.g., Miyake, et al. (1998) J. Immunol. 161:1348-1353, while human MD2 and mouse MD2 are newly discovered MD1 homologues. For a general review of LRR proteins, see, e.g., Kobe and Deisenhofer (1994) Trends Biochem. Sci. 19:412. For the role of LRR in specific immune defenses, see, e.g., Jones, et al. (1994) Science 266:789; Dixon, et al. (1996) Cell 84:451; and Baker, et al. (1997) Science 276:726.

Similar sequences for proteins in other species should also be available. The descriptions below are directed, for exemplary purposes, to the human MD1, human MD2, and rodent, e.g., mouse MD2 natural alleles described, but are likewise applicable to allelic and/or polymorphic variants, e.g., from other individuals, as well as splicing variants, e.g., natural forms, and species variants.

These genes will allow isolation of other primate or rodent genes encoding proteins related to these, further extending the family beyond the specific embodiment described. The procedure is broadly set forth below.

TABLE 1
Primate, e.g., human, HCC5 chemokine nucleic acid sequence
(SEQ ID NO: 1) and derived amino acid sequence (SEQ ID NO: 2). A
signal sequence should be found in the full length natural gene, but
based upon related genes, variable forms may be produced by different
cell types.

| | |
|---|---|
| TTGGCCTTAG GGACCAAGCT TTTATCATCG TCAGTGGGAC TTAACCTGTC TTAAAAGTGC | 60 |
| TGCTTCTCCT ACACTCGCTC AAGATCCCGA GTCAGCTGTA TTATGGCATC CTATTAGTCA | 120 |
| GGCAGCCTGT GCTTCAAGCC CGTAGTTGTA TTCATCCCCT AAAGGGGCCA TTCCGTTTGT | 180 |
| ATCATCACAT GTCCTCAGTG GGTCCATGTG TATATCAAGG ACATGATGCA GA | 232 |
| Leu Ala Leu Gly Thr Lys Leu Leu Ser Ser Ser Val Gly Leu Asn Leu | |
| 1 5 10 15 | |
| Ser Xaa Lys Cys Cys Phe Ser Tyr Thr Arg Ser Arg Ser Arg Val Ser | |
| 20 25 30 | |
| Cys Ile Met Ala Ser Tyr Xaa Ser Gly Ser Leu Cys Phe Lys Pro Val | |
| 35 40 45 | |
| Val Val Phe Ile Pro Xaa Arg Gly His Ser Val Cys Ile Ile Thr Cys | |
| 50 55 60 | |
| Pro Gln Trp Val His Val Tyr Ile Lys Asp Met Met Gln | |
| 65 70 75 | |
| LALGTKLLSS SVGLNLSxKC CFSYTRSRSR VSCIMASYxS GSLCFKPVVV FIPxRGHSVC | |
| IITCPQWVHV YIKDMMQ | |

ClustalW alignment of chemokine protein
sequences related to I-ICC5. See, e.g., human chemokine stem cell
mobilizing chemokine (CKbeta-1) : Kreider, et al. (1997) Patent WO
9715594 (SEQ ID NO: 3) is GenEank Accession number 97P-W17659;
macrophage inflammatory protein-1-gamma (NIP-1γ): Adams, et al.
(1995) Patent WO 9517092 (SEQ ID NO: 4) is GenBank Accession number
95P-R76128; human MIP-4, a chemoattractant for leukocytes: Adams, et
al. (1997) Patent WO 9634891 (SEQ ID NO: 5) is GenBank Accession
number 96P-WO7203; pituitary expressed chemokine (PGEC): Bandman, et
al. (19xx) Patent WO 9616979 (SEQ ID NO: 6) is GenBank Accession
number 96P-R95691; and HCC1: Forsmann, et al. (1998) Patent WO
9741230 (SEQ ID NO: 7) is GenBank Accession number 97P-W38171.

| | | | |
|---|---|---|---|
| HCC5 | 1 | LALGTKLLSSSVGLNLSXKCCFSYTRSRSRVSCIM | 35 |
| CKbeta-1 | 1 | TKTESSSRGPYHPSECCFTYTTYKIPRQRIM | 31 |
| MIP-1 | 1 | MKISVAAIPFFLLITIALGTKTESSSRGPYHPSECCFTYTTYKIPRQRIM | 50 |
| MIP-4 | 1 | MKISVAAIPFFLLITIALGTKTESSSRGPYHPSECCFTYTTYKIPRQRIM | 50 |
| PGEC | 1 | MKISVAAIPFFLLITIALGTKTESSSRGPYHPSECCFTYTTYKIPRQRIM | 50 |
| HCC1 chemokine | 1 | MKISVAAIPFFLLITIALGTKTESSSRGPYHPSECCFTYTTYKIPRQRIM | 50 |
| | | ** *** * ***.** . ** | |
| HCC5 | 36 | ASYXSGSLCFKPVVVFIPXRGHSVCIITCPQWVHVYIKDMMQ | 77 |
| CKbeta-1 | 32 | DYYETNSQCSKPGIVFITXRGHSVCTNPSDKWVQDYIKDMK | 72 |
| MIP-1 | 51 | DYYETNSQCSKPGIVFITKRGHSVCTNPSDKWVQDYIKDMKENTKTESSS | 100 |
| MIP-4 | 51 | DYYETNSQCSKPGIVFITKRGHSVCTNPSDKWVQDYIKDMKEN | 93 |
| PGEC | 51 | DYYETNSQCSKPGIVFITKRGHSVCTNPSDKWVQDYIKDMKEN | 93 |
| HCC1 chemokine | 51 | DYYETNSQCSKPGIVFITKRGHSVCTNPSDKWVQDYIKDMKEN | 93 |
| | | * ..* * ** .*** ****** . .**. ***** | |
| HCC5 | 78 | | 77 |
| CKbeta-1 | 73 | | 72 |
| MIP-1 | 101 | RGPYHPSECCFTYTTYKIPRQRIMDYYETNSQCSKPGIVFITX | 143 |
| MIP-4 | 94 | | 93 |
| PGEC | 94 | | 93 |
| HCC1 chemokine | 94 | | 93 |

TABLE 2
Primate, e.g., human, Dub11 (SEQ ID NO: 8 and 10) and Dub12
(SEQ ID NO: 12 and 14) deubiquitination nucleic acid sequences, and
amino acid sequences (SEQ ID NO: 9, 11, 13, and 15).

| | |
|---|---|
| Dub11 (SEQ ID NO: 8 and 9) | |
| ATG CCT TTC CCC GGC CCA CAA GCA GGT AGA TCT TCC ACT CTA AAG GAC | 48 |
| Met Pro Phe Pro Gly Pro Gln Ala Gly Arg Ser Ser Thr Leu Lys Asp | |
| 1 5 10 15 | |
| ACC ACC CCT CCA TCC CAC CAA ATA TTT GGA AGG CTC CTG GAA GAT CTC | 96 |
| Thr Thr Pro Pro Ser His Gln Ile Phe Gly arg Leu Leu Glu Asp Leu | |
| 20 25 30 | |
| CAA ATC CAA GTG TCT CCC ACT GCC CAC GGC ATT CCA GAC ACT TTT GAC | 144 |
| Gln Ile Gln Val Ser Pro Thr Ala His Gly Ile Pro Asp Thr Phe Asp | |
| 35 40 45 | |
| CCT TAC CTG GAC ATC GCC CTG GAT ATC CAG GCA GCT CAG AGT GTC CAG | 192 |
| Pro Tyr Leu Asp Ile Ala Leu Asp Ile Gln Ala Ala Gln Ser Val Gln | |
| 50 55 60 | |
| CAA GCT TTG GAA CAG TTG GTG AAG CCC GAA GAA CTC AAT GGA GAG AAT | 240 |
| Gln Ala Leu Glu Gln Leu Val Lys Pro Glu Glu Leu Asn Gly Glu Asn | |
| 65 70 75 80 | |
| GCC TAT CAT TGT GGT CTT TGT CTC CAG AGG GCG CCG GCC TCC AAG ACG | 288 |
| Ala Tyr His Cys Gly Leu Cys Leu Gln Arg Ala Pro Ala Ser Lys Thr | |
| 85 90 95 | |
| TTA ACT TTA CAC ACC TCT GCC AAG GTC CTC ATC CTT GTC TTG AAG AGA | 336 |
| Leu Thr Leu His Thr Ser Ala Lys Val Leu Ile Leu Val Leu Lys Arg | |
| 100 105 110 | |
| TTC TCC GAT GTC ACA GGC AAC AAG ATT GCC AAG AAT GTG CAA TAT CCT | 384 |
| Phe Ser Asp Val Thr Gly Asn Lys Ile Ala Lys Asn Val Gln Tyr Pro | |
| 115 120 125 | |
| GAG TGC CTT GAC ATG CAG CCA TAC ATG TCT CAG CAG AAC ACA GGA CCT | 432 |
| Glu Cys Leu Asp Met Gln Pro Tyr Met Ser Gln Gln Asn Thr Gly Pro | |
| 130 135 140 | |
| CTT GTC TAT GTC CTC TAT GCT GTG CTG GTC CAC GCT GGG TGG AGT TGT | 480 |
| Leu Val Tyr Val Leu Tyr Ala Val Leu Val His Ala Gly Trp Ser Cys | |
| 145 150 155 160 | |
| CAC AAC GGA CAT TAC TTC TCT TAT GTC AAA GCT CAA GAA GGC CAG TGG | 528 |
| His Asn Gly His Tyr Phe Ser Tyr Val Lys Ala Gln Glu Gly Gln Trp | |
| 165 170 175 | |
| TAT AAA ATG GAT GAT GCC GAG GTC ACC GCC TCT AGC ATC ACT TCT GTC | 576 |
| Tyr Lys Met Asp Asp Ala Glu Val Thr Ala Ser Ser Ile Thr Ser Val | |
| 180 185 190 | |
| CTG AGT CAA CAG GCC TAC GTC CTC TTT TAC ATC CAG AAG AGT GAA TGG | 624 |
| Leu Ser Gln Gln Ala Tyr Val Leu Phe Tyr Ile Gln Lys Ser Glu Trp | |
| 195 200 205 | |
| GAA AGA CAC AGT GAG AGT GTG TCA AGA GGC AGG GAA CCA AGA GCC CTT | 672 |
| Glu Arg His Ser Glu Ser Val Ser Arg Gly Arg Glu Pro Arg Ala Leu | |
| 210 215 220 | |
| GGC GCA GAA GAC ACA GAC AGG CGA GCA ACT CAA GGA GAG CTC AAG AGA | 720 |
| Gly Ala Glu Asp Thr Asp Arg Arg Ala Thr Gln Gly Glu Leu Lys Arg | |
| 225 230 235 240 | |
| GAC CAC CCC TGC CTC CAG GCC CCC GAG TTG GAC GAG CAC TTG GTG GAA | 768 |
| Asp His Pro Cys Leu Gln Ala Pro Glu Leu Asp Glu His Leu Val Glu | |
| 245 250 255 | |
| AGA GCC ACT CAG GAA AGC ACC TTA GAC CAC TGG AAA TTC CTT CAA GAG | 816 |
| Arg Ala Thr Gln Glu Ser Thr Leu Asp His Trp Lys Phe Leu Gln Glu | |
| 260 265 270 | |
| CAA AAC AAA ACG AAG CCT GAG TTC AAC GTC AGA AAA GTC GAA GGT ACC | 864 |
| Gln Asn Lys Thr Lys Pro Glu Phe Asn Val Arg Lys Val Glu Gly Thr | |
| 275 280 285 | |
| CTG FCCT CCC GAC GTA CTT GTG ATT CAT CAA TCA AAA TAC AAG TGT GGG | 912 |
| Leu Pro Pro Asp Val Leu Val Ile His Gln Ser Lys Tyr Lys Cys Gly | |
| 290 295 300 | |
| ATG AAG AAC CAT CAT CCT GAA CAG CAA AGC TCC CTG CTA AAC CTC TCT | 960 |
| Met Lys Asn His His Pro Glu Gln Gln Ser Ser Leu Leu Asn Leu Ser | |
| 305 310 315 320 | |
| TCG ACG ACC CCG ACA CAT CAG GAG TCC ATG AAC ACT GGC ACA CTC GCT | 1008 |
| Ser Thr Thr Pro Thr His Gln Glu Ser Met Asn Thr Gly Thr Leu Ala | |
| 325 330 335 | |
| TCC CTG CGA GGG AGG GCC AGG AGA TCC AAA GGG AAG AAC AAA CAC AGC | 1056 |
| Ser Leu Arg Gly Arg Ala Arg Arg Ser Lys Gly Lys Asn Lys His Ser | |
| 340 345 350 | |
| AAG AGG GCT CTG CTT GTG TGC CAG TG | 1082 |
| Lys Arg Ala Leu Leu Val Cys Gln | |
| 355 360 | |
| MPFPGPQAGR SSTLKDTTPP SHQIFGRLLE DLQIQVSPTA HGIPDTFDPY LDIALDIQAA | |
| QSVQQALEQL VKPEELNGEN AYHCGLCLQR APASKTLTLH TSAKVLILVL KRFSDVTGNK | |
| IAKNVQYPEC LDMQPYMSQQ NTGPLVYVLY AVLVHAGWSC HNGHYFSYVK AQEGQWYKMD | |
| DAEVTASSIT SVLSQQAYVL FYIQKSEWER HSESVSRGRE PRALGAEDTD RRATQGELKR | |
| DHPCLQAPEL DEHLVERATQ ESTLDHWKFL QEQNKTKPEF NVRKVEGTLP PDVLVIHQSK | |
| YKCGMKNHHP EQQSSLLNLS STTPTHQESM NTGTLASLRG RARRSKGKNK HSKRALLVCQ | |
| updated Dub11 sequence (SEQ ID NO: 10 and 11): | |
| atg gag gac gac tca ctc tac ttg gga ggt gag tgg cag ttc aac cac | 48 |
| Met Glu Asp Asp Ser Leu Tyr Leu Gly Gly Glu Trp Gln Phe Asn His | |
| 1 5 10 15 | |
| ttt tca aaa ctc aca tct tct cgg cca gat gca gct ttt gct gaa atc | 96 |
| Phe Ser Lys Leu Thr Ser Ser Arg Pro Asp Ala Ala Phe Ala Glu Ile | |
| 20 25 30 | |
| cag cgg act tct ctc cct gag aag tca cca ctc tca tct gag gcc cgt | 144 |
| Gln Arg Thr Ser Leu Pro Glu Lys Ser Pro Leu Ser Ser Glu Ala Arg | |
| 35 40 45 | |
| gtc gac ctc tgt gat gat ttg gct cct gtg gca aga cag ctt gct ccc | 192 |
| Val Asp Leu Cys Asp Asp Leu Ala Pro Val Ala Arg Gln Leu Ala Pro | |
| 50 55 60 | |
| agg gag aag ctt cct ctg agt agc agg aga cct gct gcg gtg ggg gct | 240 |
| Arg Glu Lys Leu Pro Leu Ser Ser Arg Arg Pro Ala Ala Val Gly Ala | |
| 65 70 75 80 | |
| ggg ctc cag aat atg gga aat acc tgc tac gag aac gct tcc ctg cag | 288 |
| Gly Leu Aln Asn Met Gly Asn Thr Cys Tyr Glu Asn Ala Ser Leu Gln | |
| 85 90 95 | |
| tgc ctg aca tac aca ccg ccc ctt gcc aac tac atg ctg tcc cgg gag | 336 |
| Cys Leu Thr Tyr Thr Pro Pro Leu Ala Asn Tyr Met Leu Ser Arg Glu | |
| 100 105 110 | |
| cac tct caa aca tgt cag cgt ccc aag tgc tgc atg ctc tgt act atg | 384 |
| His Ser Gln Thr Cys Gln Arg Pro Lys Cys Cys Met Leu Cys Thr Met | |
| 115 120 125 | |
| caa gct cac atc aca tgg gcc ctc cac agt cct ggt cat gtc atc cag | 432 |
| Gln Ala His Ile Thr Trp Ala Leu His Ser Pro Gly His Val Ile Gln | |
| 130 135 140 | |
| ccc tca cag gca ttg gct gct ggc ttc cat aga ggc aag cag gaa gat | 480 |
| Pro Ser Gln Ala Leu Ala Ala Gly Phe His Arg Gly Lys Gln Glu Asp | |
| 145 150 155 160 | |
| gcc cat gaa ttt ctc atg ttc act gtg gat gcc atg aaa aag gca tgc | 528 |
| Ala His Glu Phe Leu Met Phe Thr Val Asp Ala Met Lys Lys Ala Cys | |
| 165 170 175 | |
| ctt ccc ggc cac aag cag gta gat cat cac tct aag gac acc acc ctc | 576 |
| Leu Pro Gly His Lys Gln Val Asp His His Ser Lys Asp Thr Thr Leu | |
| 180 185 190 | |
| atc cac caa ata ttt gga ggc tgc tgg aga tct caa atc aag tgt ctc | 624 |
| Ile His Gln Ile Phe Gly Gly Cys Trp Arg Ser Gln Ile Lys Cys Leu | |
| 195 200 205 | |
| cac tgc cac ggg att cca gac act ttt gac cct tac ctg gac atc gcc | 672 |
| His Cys His Gly Ile Pro Asp Thr Phe asp Pro Tyr Leu Asp Ile Ala | |
| 210 215 220 | |
| ctg gat atc cag gca gct cag agt gtc aag caa gct ttg gaa cag ttg | 720 |
| Leu Asp Ile Gln Ala Ala Gln Ser Val Lys Gln Ala Leu Glu Gln Leu | |
| 225 230 235 240 | |
| gtg aag ccc gaa gaa ctc aat gga gag aat gcc tat cat tgt ggt ctt | 768 |
| Val Lys Pro Glu Glu Leu Asn Gly Glu Asn Ala tyr His Cys Gly Leu | |
| 245 250 255 | |
| tgt ctc cag agg gcg ccg gcc tcc aag acg tta act tta cac act tct | 816 |
| Cys Leu Gln Arg Ala Pro Ala Ser Lys Thr Leu Thr Leu His Thr Ser | |
| 260 265 270 | |
| gcc aag gtc ctc atc ctt gta ttg aag aga ttc tcc gat gtc aca ggc | 864 |
| Ala Lys Val Leu Ile Leu Xaa Leu Lys Arg Phe Ser Asp Val Thr Gly | |
| 275 280 285 | |
| aac aaa ctt gcc aag aat gtg caa tat cct gag tgc ctt gac atg cag | 912 |
| Asn Lys Leu Ala Lys Asn Val Gln Tyr Pro Glu Cys Leu Asp Met Gln | |
| 290 295 300 | |
| cca tac atg tct cag cag aac aca gga cct ctt gtc tat gtc ctc tat | 960 |
| Pro Tyr Met Ser Gln Gln Asn Thr Gly Pro Leu Val Tyr Val Leu Tyr | |
| 305 310 315 320 | |
| gct gtg ctg gtc cac gct ggg tgg agt tgt cac aac gga cat tac ttc | 1008 |
| Ala Val Leu Val His Ala Gly Trp Ser Cys His Asn Gly His Tyr Phe | |
| 325 330 335 | |
| tct tat gtc aaa gct caa gaa ggc cag tgg tat aaa atg gat gat gcc | 1056 |
| Ser Tyr Val Lys Ala Gln Glu Gly Gln Trp Tyr Lys Met Asp Asp Ala | |
| 340 345 350 | |
| gag gtc acc gcc tct agc atc act tct gtc ctg agt caa cag gcc tac | 1104 |
| Glu Val Thr Ala Ser Ser Ile Thr Ser Val Leu Ser Gln Gln Ala Tyr | |
| 355 360 365 | |
| gtc ctc ttt tac atc cag aag agt gaa tgg gaa aga cac agt gag agt | 1152 |
| Val Leu Phe Tyr Ile Gln Lys Ser Glu Trp Glu Arg His Ser Glu Ser | |
| 370 375 380 | |
| gtg tca aga ggc agg gaa cca aga gcc ctt ggc gca gaa gac aca gac | 1200 |
| Val Ser Arg Gly Arg Glu Pro Arg Ala Leu Gly Ala Glu Asp Thr Asp | |
| 385 390 395 400 | |
| agg cga gca acg caa gga gag ctc aag aga gac cac ccc tgc ctc cag | 1248 |
| Arg Arg Ala Thr Gln Gly Glu Leu Lys Arg Asp His Pro Cys Leu Gln | |
| 405 410 415 | |
| gcc ccc gag ttg gac gag cac ttg gtg gaa aga gcc act cag gaa agc | 1296 |
| Ala Pro Glu Leu Asp Glu His Leu Val Glu Arg Ala Thr Gln Glu Ser | |
| 420 425 430 | |
| acc tta gac cac tgg aaa ttc ctt caa gag caa aac aaa acg aag cct | 1344 |
| Thr Leu Asp His Trp Lys Phe Leu Gln Glu Gln Asn Lys Thr Lys Pro | |
| 435 440 445 | |
| gag ttc aac gtc aga aaa gtc gaa ggt acc ctg cct ccc gac gta ctt | 1392 |
| Glu Phe Asn Val Arg Lys Val Glu Gly Thr Leu Pro Pro Asp Val Leu | |
| 450 455 460 | |
| gtg att cat caa tca aaa tac aag tgt ggg ata aag aac cat cat cct | 1440 |
| Val Ile His Gln ser Lys Tyr Lys Cys Gly Met Lys Asn His His Pro | |
| 465 470 475 480 | |
| gaa cag caa agc tcc ctg cta aac ctc tct tcg acg acc ccg aca cat | 1488 |
| Glu Gln Gln Ser Ser Leu Leu Asn Leu Ser Ser Thr Thr Pro Thr His | |
| 485 490 495 | |
| cag gag tcc atg aac act ggc aca ctc gct tcc ctg cga ggg agg gcc | 1536 |
| Gln Glu Ser Met Asn Thr Gly Thr Leu Ala Ser Leu Arg Gly Arg Ala | |
| 500 505 510 | |
| agg aga tcc aaa ggg aag aac aaa cac agc aag agg gct ctg ctt gtg | 1584 |
| Arg Arg Ser Lys Gly Lys Asn Lys His Ser Lys Arg Ala Leu Leu Val | |
| 515 520 525 | |
| tgc cag tgatctcagt ggaagtaccg acccacacgt aggggtgcac acacacacgc | 1640 |
| Cys Gln | |
| 530 | |
| acacacacag acacacacat aactacaccc agaagcgcgc tga | 1683 |
| MEDDSLYLGG EWQFNHFSKL TSSRPDAAFA EIQRTSLPEK SPLSSEARVD LCDDLAPVAR | |
| QLAPREKLPL SSRRPAAVGA GLQNMGNTCY ENASLQCLTY TPPLANYMLS REHSQTCQRP | |
| KCCMLCTMQA HITWALHSPG HVIQPSQALA AGFHRGKQED AHEFLMFTVD AMKKACLPGH | |
| KQVDHHSKDT TLIHQIFGGC WRSQIKCLHC HGIPDTFDPY LDIALDIQAA QSVKQALEQL | |
| VKPEELNGEN AYHCGLCLQR APASKTLTLH TSAKVLILxL KRFSDVTGNK LAKNVQYPEC | |
| LDMQPYMSQQ NTGPLVYVLY AVLVHAGWSC HNGHYFSYVK AQEGQWYKMD DAEVTASSIT | |
| SVLSQQAYVL FYIQKSEWER HSESVSRGRE PRALGAEDTD RRATQGELKR DHPCLQAPEL | |
| DEHLVERATQ ESTLDHWKFL QEQNKTKPEF NVAKVEGTLP PDVLVIHQSK YKCGMKNHHP | |
| EQQSSLLNLS STTPTHQESM NTGTLASLRG RARRSKGKNK HSKRALLVCQ | |
| Dub12 (SEQ ID NO: 12 and 13): | |
| ATG GCT GTG CCA AGT TGG ATC GTC AAA CGC AGG CTA CTA CCT TGG TCC | 48 |
| Met Ala Val Pro Ser Trp Ile Val Lys Arg Arg Leu Leu Pro Trp Ser | |
| 1 5 10 15 | |
| ATC AAA TTT TTG GAG GGT ATC TCA GAT CAC GGC GTG AAG TGC TCC GTG | 96 |
| Ile Lys Phe Leu Glu Gly Ile Ser Asp His Gly Val Lys Cys Ser Val | |
| 20 25 30 | |
| TGC AAG AGC GTC TCG GAC ACC TAC GAC CCC TAC TTG GAC GTC GCG CTG | 144 |
| Cys Lys Ser Val Ser Asp Thr Tyr Asp Pro Tyr Leu Asp Val Ala Leu | |
| 35 40 45 | |
| GAG ATC CGG CAA GCT GCG AAT ATT GTG CGT GCT CTG GAA CTT TTT GTG | 192 |
| Glu Ile Arg Gln Ala Ala Asn Ile Val Arg Ala Leu Glu Leu Phe Val | |
| 50 55 60 | |
| AAA GCA GAT GTC CTG AGT GGA GAG AAT GCC TAC ATG TGT GCT AAA TGC | 240 |
| Lys Ala Asp Val Leu Ser Gly Glu Asn Ala Tyr Met Cys Ala Lys Cys | |
| 65 70 75 80 | |
| AAG AAG AAG GTT CCA GCC AGC AAG CGC TTC ACC ATC CAC AGA ACA TCC | 288 |
| Lys Lys Lys Val Pro Ala Ser Lys Arg Phe Thr Ile His Arg Thr Ser | |
| 85 90 95 | |
| AAC GTC TTA ACC CTT TCC CTC AAG CGC TTT GCC AAC TTC AGC GGG GGG | 336 |
| Asn Val Leu Thr Leu Ser Leu Lys Arg Phe Ala Asn Phe Ser Gly Gly | |
| 100 105 110 | |
| AAG ATC ACC AAG GAT GTA GGC TAT CCG GAA TTC CTC AAC ATA CGT CCG | 184 |
| Lys Ile Thr Lys Asp Val Gly Tyr Pro Glu Phe Leu Asn Ile Arg Pro | |
| 115 120 125 | |
| TAT ATG TCC CAG AAT AAT GGT GAT CCT GTC ATG TAT GGA CTC TAT GCT | 432 |
| Tyr Met Ser Gln Asn Asn Gly Asp Pro Val Mat Tyr Gly Leu Tyr Ala | |
| 130 135 140 | |
| GTC CTG GTG CAC TCG GGC TAC AGC TGC CAT GCC GGG CAC TAT TAC TGC | 480 |
| Val Leu Val His Ser Gly Tyr Ser Cys His Ala Gly His Tyr Tyr Cys | |
| 145 150 155 160 | |
| TAC GTG AAG GCA AGC AAT GGA CAG TGG TAC CAG ATG AAT GAT TCC TTG | 528 |
| Tyr Val Lys Ala Ser Asn Gly Gln Trp Tyr Gln Met Asn Asp Ser Leu | |
| 165 170 175 | |
| GTC CCA TTC CAG CAA CGT CCA AGT TGG TTT CTG AAA CCA GCA GGC CTA | 576 |
| Val Pro Phe Gln Gln Arg Pro Ser Trp Phe Leu Lys Pro Ala Gly Leu | |
| 180 185 190 | |
| AGT GGC TTG TTC TCA TCG GCG AAT TTC CAG GCT CTC AAG AAA AAT TCC | 624 |
| Ser Gly Leu Phe Ser Ser Ala Asn Phe Gln Ala Leu Lys Lys Asn Ser | |
| 195 200 205 | |
| CGA AGGAGCC TCC ATT TTC CAG GAA CAG GTT CCT TCC TCC CCT TCC CGG | 672 |
| Arg Arg Ala Ser Ile Phe Gln Glu Gln Val Pro Ser Ser Pro Ser Arg | |
| 210 215 220 | |
| GCG GCC CGA ATT GTG AAT TCC AGA TTC ATT CCC AGC AGG AAC CTC GGC | 720 |
| Ala Ala Arg Ile Val Asn Ser Arg Phe Ile Pro Ser Arg Asn Leu Gly | |
| 225 230 235 240 | |
| AAT GGG GAT TAT TTT | 735 |
| Asn Gly Asp Tyr Phe | |
| 245 | |
| MAVPSWIVKR RLLPWSIKFL EGISDHGVKC SVCKSVSDTY DPYLDVALEI RQAANIVRAL | |
| ELFVKADVLS GENAYMCAKC KKKVPASKRF TIHRTSNVLT LSLKRFANFS GGKITKDVGY | |
| PEFLNIRPYM SQNNGDPVMY GLYAVLVHSG YSCHAGHYYC YVKASNGQWY QMNDSLVPFQ | |
| QRPSWFLKPA GLSGLFSSAN FQALKKNSRR ASIFQEQVPS SPSRAARIVN SRFIPSRNLG | |
| NGDYF | |
| updated Dub12 sequence (SEQ ID NO: 14 and 15): | |
| atg cag aaa gcc tgc ctg aat ggc tgt gcc aag ttg gat cgt caa acg | 48 |
| Met Gln Lys Ala Cys Leu Asn Gly Cys Ala Lys Leu Asp Arg Gln Thr | |
| 1 5 10 15 | |
| cag gct act acc ttg gtc cat caa att ttt gga ggg tat ctc aga tca | 96 |
| Gln Ala Thr Thr Leu Val His Gln Ile Phe Gly Gly Tyr Leu Arg Ser | |
| 20 25 30 | |
| cgc gtg aag tgc tcc gtg tgc aag agc gtc tcg gac acc tac gac ccc | 144 |
| Arg Val Lys Cys Ser Val Cys Lys Ser Val Ser Asp Thr Tyr Asp Pro | |
| 35 40 45 | |
| tac ttg gac gtc gcg ctg gag atc cgg caa gct gcg aat att gtg cgt | 192 |
| Tyr Leu Asp Val Ala Leu Glu Ile Arg Gln Ala Ala Asn Ile Val Arg | |
| 50 55 60 | |
| gct ctg gaa ctt ttt gtg aaa gca gat gtc ctg agt gga gag aat gcc | 240 |
| Ala Leu Glu Leu Phe Val Lys Ala Asp Val Leu Ser Gly Glu Asn Ala | |
| 65 70 75 80 | |
| tac atg tgt gct aaa tgc aag aag aag gtt cca gcc agc aag cgc ttc | 288 |
| Tyr Met Cys Als Lys Cys Lys Lys Lys Val Pro Ala Ser Lys Arg Phe | |
| 85 90 95 | |
| acc atc cac aga aca tcc aac gtc tta acc ctt tcc ctc aag cgc ttt | 336 |
| Thr Ile His Arg Thr Ser Asn Val Leu Thr Leu Ser Leu Lys Arg Phe | |
| 100 105 110 | |
| gcc aac ttc agc ggg ggg aag atc acc aag gat gta ggc tat ccg gaa | 384 |
| Ala Asn Phe Ser Gly Gly Lys Ile Thr Lys Asp Val Gly Tyr Pro Glu | |
| 115 120 125 | |
| ttc ctc aac ata cgt ccg tat atg tcc cag aat aat ggt gat cct gtc | 432 |
| Phe Leu Asn Ile Arg Pro Tyr Met Ser Gln Asn Asn Gly Asp Pro Val | |
| 130 135 140 | |
| atg tat gga ctc tat gct gtc ctg gtg cac tcg ggc tac agc tgc cat | 480 |
| Met Tyr Gly Leu Tyr Ala Val Leu Val His Ser Gly Tyr Ser Cys His | |
| 145 150 155 160 | |
| gcc ggg cac tat tac tgc tac gtg aag gca agc aat gga cag tgg tac | 528 |
| Ala Gly His Tyr Tyr Cys Tyr Val Lys Ala Ser Asn Gly Gln Trp Tyr | |
| 165 170 175 | |
| cag atg aat gat tcc ttg gtc cat tcc agc aac gtc aag gtg gtt ctg | 576 |
| Gln Met Asn Asp Ser Leu Val His Ser Ser Asn Val Lys Val Val Leu | |
| 180 185 190 | |
| aac cag cag gcc tac gtg ctg ttc tat ctg cga att cca ggc tct aag | 624 |
| Asn Gln Gln Ala Tyr Val Leu Phe Tyr Leu Arg Ile Pro Gly Ser Lys | |
| 195 200 205 | |
| aaa agt ccc gag ggc ctc atc tcc agg aca ggc tcc tcc tcc ctt ccc | 672 |
| Lys Ser Pro Glu Gly Leu Ile Ser Arg Thr Gly Ser Ser Ser Leu Pro | |
| 210 215 220 | |
| ggc cgc ccg agt gtg att cca gat cac tcc aag aag aac atc ggc aat | 720 |
| Gly Arg Pro Ser Val Ile Pro Asp His Ser Lys Lys Asn Ile Gly Asn | |
| 225 230 235 240 | |
| ggg att att tcc tcc cca ctg act gga aag cga caa gac tct ggg acg | 768 |
| Gly Ile Ile Ser SEr Pro Leu Thr Gly Lys Arg Gln Asp Ser Gly Thr | |
| 245 250 255 | |
| atg aag aag ccg cac acc act gaa gag att ggt gtg ccc ata tcc agg | 816 |
| Met Lys Lys Pro His Thr Thr Glu Glu Ile Gly Val Pro Ile Ser Arg | |
| 260 265 270 | |
| aat ggc tcc acc ctg ggc ctg aag tcc cag aac ggc tgc att cct cca | 864 |
| Asn Gly Ser Thr Leu Gly Leu Lys Ser Gln Asn Gly Cys Ile Pro Pro | |
| 275 280 285 | |
| aag ctg ccc tcg ggg tcc cct tcc ccc aaa ctc tcc cag aca ccc aca | 912 |
| Lys Leu Pro SEr Gly Ser Pro Ser Pro Lys Leu Ser Gln Thr Pro Thr | |
| 290 295 300 | |
| cac atg cca acc atc cta gac gac cct gga aag aag gtg aag aag cca | 960 |
| His Met Pro Thr Ile Leu Asp Asp Pro Gly Lys Lys Val Lys Lys Pro | |
| 305 310 315 320 | |
| gct cct cca cag cac ttt tcc ccc aga act gct cag ggg ctg cct ggg | 1008 |
| Ala Pro Pro Aln His Phe Ser Pro Arg Thr Ala Gln Gly Leu Pro Gly | |
| 325 330 335 | |
| acc agc aac tcg aat agc agc aga tct ggg agc caa agg cag ggc tcc | 1056 |
| Thr Ser Asn Ser Asn Ser Ser ARg Ser Gly Ser Gln Arg Gln Gly Ser | |
| 340 345 350 | |
| tgg gac agc agg gat gtt gtc ctc tct acc tca cct aag ctc ctg gct | 1104 |
| Trp Asp Ser Arg Asp Val Val Leu Ser Thr Ser Pro Lys Leu Leu Ala | |
| 355 360 365 | |
| aca gcc act gcc aac ggg cat ggg ctg aag ggg aac gac gag agc gct | 1152 |
| Thr Ala Thr Ala Asn Gly His Gly Leu Lys Gly Asn Asp Glu Ser Ala | |
| 370 375 380 | |
| ggc ctc gac agg agg ggc tcc agc agc tcc agc cca gag cac tcg gcc | 1200 |
| Gly Leu Asp Arg Arg Gly Ser Ser Ser Ser Ser Pro Glu His Ser Ala | |
| 385 390 395 400 | |
| agc agc gac tcc acc aag gcc ccc cag acc ccc agg agt gga gcg gcc | 1248 |
| Ser Ser Asp Ser Thr Lys Ala Pro Gln Thr Pro Arg Ser Gly Ala Ala | |
| 405 410 415 | |
| cat ctc tgc gat tct cag gaa acg aac tgt tcc acc gct ggc cac tcc | 1296 |
| His Leu Cys Asp Ser Gln Glu Thr Asn Cys Ser Thr Ala Gly His Ser | |
| 420 425 430 | |
| aaa acg ccg cca agt gga gca gat tct aag acg gtg aag ctg aag tcc | 1344 |
| Lys Thr Pro Pro Ser Gly Ala Asp Ser Lys Thr Val Lys Leu Lys Ser | |
| 435 440 445 | |
| cct gtc ctg agc aac acc acc act gag cct gca agc acc atg tct cct | 1392 |
| Pro Val Leu Ser Asn Thr Thr Thr Glu Pro Ala Ser Thr Met Ser Pro | |
| 450 455 460 | |
| cca cca gcc aaa aaa ctg gcc ctt tct gcc aag aag gcc agc acc ctg | 1440 |
| Pro Pro Ala Lys Lys Leu Ala Leu Ser Ala Lys Lys Ala Ser Thr Leu | |
| 465 470 475 480 | |
| tgg agg gcg acc ggc aat gac ctc cgt cca cct ccc ccc tca cca tcc | 1488 |
| Trp Arg Ala Thr Gly Asn Asp Leu Arg Pro Pro Pro Pro Ser Pro Ser | |
| 485 490 495 | |
| tcc gac ctc acc cac ccc atg aaa acc tct cac ccc gtc gtt gcc tcc | 1536 |
| Ser Asp Leu Thr His Pro Met Lys Thr Ser His Pro Val Val Ala Ser | |
| 500 505 510 | |
| act tgg ccc gtc cat aga gcc agg gct gtg tca cct gct ccc caa tca | 1584 |
| Thr Trp Pro Val His Arg Ala Arg Ala Val Ser Pro Ala Pro Gln Ser | |
| 515 520 525 | |
| tcc agc cgc ctg caa ccc ccc ttc agc ccc cac ccc aca ttg ctg tcc | 1632 |
| Ser Ser Arg Leu Gln Pro Pro Phe Ser Pro His Pro Thr Leu Leu Ser | |
| 530 535 540 | |
| agt acc ccc aag ccc cca ggg acg tca gaa cca cgg agc tgc tcc tcc | 1680 |
| Ser Thr Pro Lys Pro Pro Gly Thr Ser Glu Pro Arg Ser Cys Ser Ser | |
| 545 550 555 | 560 |
| atc tcg acg gcg ctg cct cag gtc aac gag gac ctt gtg tct ctt cca | 1728 |
| Ile Ser Thr Ala Leu Pro Gln Val Asn Glu Asp Leu Val Ser Leu Pro | |
| 565 570 575 | |
| cac cag ttg cca gag gcc agt gag ccc ccc cag agc ccc tct gag aag | 1776 |
| His Gln Leu Pro Glu Ala Ser Alu Pro Pro Gln Ser Pro Ser Glu Lys | |
| 580 585 590 | |
| agg aaa aag acc ttt gtg gga gag ccg cag agg ctg ggc tca gag acg | 1824 |
| Arg Lys Lys Thr Phe Val Gly Glu Pro Gln Arg Leu Gly Ser Glu Thr | |
| 595 600 605 | |
| cgc ctc cca cag cac atc agg gag gcc act gcg gct ccc cac ggg aag | 1872 |
| Arg Leu Pro Gln His Ile Arg Glu Ala Thr Ala Ala Pro His Gly Lys | |
| 610 615 620 | |
| agg aag agg aag aag aag aag cgc ccg gag gac aca gct gcc agc gcc | 1920 |
| Arg Lys Arg Lys Lys Lys Lys Arg Pro Glu Asp thr Ala Ala Ser Ala | |
| 625 630 635 640 | |
| ctg cag gag ggg cag aca cag aga cag cct ggg agc ccc atg tac agg | 1968 |
| Leu Gln Glu Gly Gln Thr Gln Arg Gln Pro Gly Ser Pro Met Tyr Arg | |
| 645 650 655 | |
| agg gag ggc cag gca cag ctg ccc gct gtc aga cgg cag gaa gat ggc | 2016 |
| Arg Glu Gly Gln Ala Gln Leu Pro Ala Val Arg Arg Gln Glu Asp Gly | |
| 660 665 670 | |
| aca cag cca cag gtg aat ggc cag cag gtg gga tgt gtt acg gac ggc | 2064 |
| Thr Gln Pro Gln Val Asn Gly Gln Gln Val Gly Cys Val Thr Asp Gly | |
| 675 680 685 | |
| cac cac gcg agc agc agg aag cgg agg agg aaa gga gca gaa ggt ctt | 2112 |
| His His Ala Ser Ser Arg Lys Arg Arg Arg Lys Gly Ala Glu Gly Leu | |
| 690 695 700 | |
| ggt gaa gaa ggc ggc ctg cac cag gac cca ctt cgg cac agc tgc tct | 2160 |
| Gly Glu Glu Gly Gly Leu His Gln Asp Pro Leu Arg His Ser Cys Ser | |
| 705 710 715 720 | |
| ccc atg ggt gat ggt gat cca gag gcc atg gaa gag tct cca agg aaa | 2208 |
| Pro Met Gly Asp Gly Asp Pro Glu Ala Met Glu Glu Ser Pro Arg Lys | |
| 725 730 735 | |
| aag aaa aaa aaa aaa aac tcg agg ggg ggc ccg gta | 2244 |
| Lys Lys Lys Lys Lys Asn Ser Arg Gly Gly Pro Val | |
| 740 745 | |
| MQKACLNGCA KLDRQTQATT LVHQIFGGYL RSRVKCSVCK SVSDTYDPYL DVALEIRQAA | |
| NIVRALELFV KADVLSGENA YMCAKCKKKV PASKRFTIHR TSNVLTLSLK RFANFSGGKI | |
| TKDVGYPEFL NIRPYMSQNN GDPVMYGLYA VLVHSGYSCH AGHYYCYVKA SNGQWYQMND | |
| SLVHSSNVKV VLNQQAYVLF YLRIPGSKKS PEGLISRTGS SSLPGRPSVI PDHSKKNIGN | |
| GIISSPLTGK RQDSGTMKKP HTTEEIGVPI SRNGSTLGLK SQNGCIPPKL PSGSPSPKLS | |
| QTPTHMPTIL DDPGKKVKKP APPQHFSPRT AQGLPGTSNS NSSRSGSQRQ GSWDSRDVVL | |
| STSPKLLATA TANGHGLKGN DESAGLDRRG SSSSSPEHSA SSDSTKAPQT PRSGAAHLCD | |
| SQETNCSTAG HSKTPPSGAD SKTVKLKSPV LSNTTTEPAS TMSPPPAKKL ALSAKKASTL | |
| WRATGNDLRP PPPSPSSDLT HPMKTSHPVV ASTWPVHRAR AVSPAPQSSS RLQPPFSPHP | |
| TLLSSTPKPP GTSEPRSCSS ISTALPQVNE DLVSLPHQLP EASEPPQSPS EKRKKTFVGE | |
| PQRLGSETRL PQHIREATAA PHGKRKRKKK KRPEDTAASA LQEGQTQRQP GSPMYRREGQ | |
| AQLPAVRRQE DGTQPQVNGQ QVGCVTDGHH ASSRKRRRKG AEGLGEEGGL HQDPLRHSCS | |
| PMGDGDPEAM EESPRKKKKK KNSRGGPV | |

ClustalW alignment of deubiquitinating protein
sequences related to Dub11 and Dub12. See e.g., Zhu, et al. (1996)
Proc. Natl. Acad. Sci. USA 93:3275-3279 (SEQ ID NO: 16) is GenBank
Accession number U41636; and Zhu, et al. (1997) J. Biol. Chem.
272:51-57 (SEQ ID NO: 17) is GenBank Accession number U70368.

| | | | |
|---|---|---|---|
| Dub1 | 1 | MVVALSFPEADPALSSPDAPELHQDEAQVVEELTVNGKHSLSWESPQGPG | 50 |
| Dub11 | 1 | | 0 |
| Dub12 | 1 | | 0 |
| Dub2 | 1 | MVVSLSFPEADPALSSPGAQQLHQDEAQVVVELTANDKPSLSWECPQGPG | 50 |
| Dub1 | 51 | CGLQNTGNSCYLNAALQCLTHTPPLADYMLSQEHSQTCCSPEGCKLCAME | 100 |
| Dub11 | 1 | | 0 |
| Dub12 | 1 | | 0 |
| Dub2 | 51 | CGLQNTGNSCYLNAALQCLTHTPPLADYMLSQEYSQTCCSPEGCKMCAME | 100 |
| Dub1 | 101 | ALVTQSLLHSHSGDVMKPSHILTSAFHKHQQEDAHEFLMFTLETMHESCL | 150 |
| Dub11 | 1 | MPFPG------------------------------- | 5 |
| Dub12 | 1 | MAVPS------------------------------- | 5 |
| Dub2 | 101 | AHVTQSLLHSHSGDVMKPSQILTSAFHKHQQEDAHEFLMFTLETMHESCL | 150 |
| | | . * | |
| Dub1 | 151 | QVHRQSKPTSEDSSPIHDIFGGWWRSQIKCLLCQGTSDTYDRFLDIPLDI | 200 |
| Dub11 | 6 | QAGRSS--TLKDTTPSHQIFGRLLEDLQIVSPTAHGIDTFDPYLDIALDI | 53 |
| Dub12 | 6 | WIVKRR------LLPWSKFLEGISDHGVKCSVCKSVSDTYDPYLDVALEI | 49 |
| Dub2 | 151 | QVHRQSEPTSEDSSPIHDIFGGLWRSQIKCLHCQGTSDTYDRFLDVPLDI | 200 |
| | | . * . **.* .**. *.* | |
| Dub1 | 201 | SSAQSVKQALWDTEKSEELCGDNAYYCGKCRQKMPASKTLHVHIAPKVLM | 250 |
| Dub11 | 54 | QAAQSV-QALEQLVKPEELNGENAYHCG-CLQRAPASKTLTLHTSAKVLI | 101 |
| Dub12 | 50 | RQAANIVRALELFVK-DVLSGENAYMCAKCKKKVPASKRFTIHRTSNVLT | 98 |
| Dub2 | 201 | SSAQSVNQALWDTEKSEELRGENAYYCGRCRQKMPASKTLHIHSAPKVLL | 250 |
| | | * . .** * . * *.*** * * .. **** .* . ** | |
| Dub1 | 251 | VVLNRFSAFTGNKLDRKVSYPEFLDLKPYLSEPTGGPLPYALYAVLVHDG | 300 |
| Dub11 | 102 | LVLKRFSDVTGNKIAKNVQYPECLDMQPYMSQQNTGPLVYVLYAVLVHAG | 151 |
| Dub12 | 99 | LSLKRFANFSGGKITKDVGYPEFLNIRPYMSQNNGDPVMYGLYAVLVHSG | 148 |
| Dub2 | 251 | LVLKRFSAFMGNKLDRKVSYPEFLDLKPYLSQPTGGPLPYALYAVLVHEG | 300 |
| | | . * **. *.*. . * *** * ..**.*. . *. * ******* * | |
| Dub1 | 301 | ATSHSGHYFCCVKAGHGKWYKMDDTKVTRCDVTSVLNENAYVLFYVQQAN | 350 |
| Dub11 | 152 | WSCHNGHYFSYVKAQEGQWYKMDDAEVTASSITSVLSQQAYVLFYIQKSE | 201 |
| Dub12 | 149 | YSCHAGHYYCYVKASNGQWYQMNDSLVP-----------------FQQ-- | 179 |
| Dub2 | 301 | ATCHSGHYFSYVKARHGAWYKMDDTKVTSCDVTSVLNENAYVLFYVQQTD | 350 |
| | | ..* ***.. *** * **.* *. * *. | |
| Dub1 | 351 | LKQVSIDMPEGRINEVLDPEYQLKKSRRKKHKKKSPFTEDLGEPCENRDK | 400 |
| Dub11 | 202 | WERHSESVSRGREPRALGAEDTDRRATQGELKRDHPCLQAP-ELDEHLVE | 250 |
| Dub12 | 180 | --RPSWFLKPAGLSGLFSANFQALK------------------KNSRR-- | 207 |
| Dub2 | 351 | LKQVSIDMPEGRVHEVLDPEYQLKKSRRKKHKKKSPCTEDAGEPCKNREK | 400 |
| | | . * . | |
| Dub1 | 401 | RAIKETSLGKGKVLQEVNHKKAGQKHGNTKLMPQKQ-------------- | 436 |
| Dub11 | 251 | RATQESTLDHWKFLQE-------------------Q-------------- | 267 |
| Dub12 | 208 | -----ASIFQEQVPSS---------------------------------- | 218 |
| Dub2 | 401 | RATKETSLGEGKVZQEKNHKKAGQKHENTKLVPQEQNHQKLGQKHRINEI | 450 |
| | | ... . | |
| Dub1 | 437 | -----NHQKAGQNLRNTEVELDLPADAIVIHQPRSTANWGRDSPDKENQP | 481 |
| Dub11 | 268 | -----NKTKPEFNVR--KVEGTLPPDVLVIHQSKYKCGMKNHHPEQQSSL | 310 |
| Dub12 | 219 | ------SR-AARIVN------------------------SRFIP------ | 231 |
| Dub2 | 451 | LPQEQNHQKAGQSLRNTEGELDLPADAIVIHLLRSTENWGRDAPDKENQP | 500 |
| | | . * | |
| Dub1 | 482 | LHNADRLLTSQGPVNTWQLCRQEGRRRSKKGQNKNKQCQRLLLVC | 526 |
| Dub11 | 311 | LNLSSTTPTHQESMNTGTLASLRGRARRSKG--KNKHSKRALLVCQ | 354 |
| Dub12 | 232 | --------------------------RN--G-----NGDYF | 239 |
| Dub2 | 501 | WHNADRLLTSQDPVNTGQLCRQEGRRRSKKGKNKNKQGQRLLLVC | 545 |
| | | * * | |

TABLE 3

| | |
|---|---|
| MD1 and MD2 sequences. Primate, e.g., human, MD1 (SEQ ID | |
| NO: 18 and 19); primate, e.g., human, MD2 (SEQ ID NO: 20 and 21); | |
| updated primate, e.g., human, MD2 (SEQ ID NO: 22 and 23); rodent, | |
| e.g., mouse, MD2 (SEQ ID NO: 24 and 25) and splice variant (SEQ ID | |
| NO: 26). | |
| primate, e.g., human, MD1 (SEQ ID NO: 18 and 19): | |
| GGCACGAGCC CACC ATG AAG GGT TTC ACA GCC ACT CTC TTC CTC TGG ACT | 50 |
| Met Lys Gly Phe Thr Ala Thr Leu Phe Leu Trp Thr | |
| 1 5 10 | |
| CTC ATT TTT CCC AGC TGC AGT GGA GGC GGC GGT GGG AAA GCC TGG CCC | 98 |
| Leu Ile Phe Pro Set Cys Ser Gly Gly Gly Gly Gly Lys Ala Trp Pro | |
| 15 20 25 | |
| ACA CAC GTG GTC TGT AGC GAC AGC CGC TTG GAA GTG CTC TAC CAG AGT | 146 |
| Thr His Val Val Cys Ser Asp Ser Arg Leu Glu Val Leu Tyr Gln Ser | |
| 30 35 40 | |
| TGC GAT CCA TTA CAA GAT TTT GGC TTT TCT GTT GAA AAG TGT TCC AAG | 194 |
| Cys Asp Pro Leu Gln Asp Phe Gly Phe Ser Val Glu Lys Cys Ser Lys | |
| 45 50 55 60 | |
| CAA TTA AAA TCA AAT ATC AAC ATT AGA TTT GGA ATT ATT CTG AGA GAG | 242 |
| Gln Leu Lys Ser Asn Ile Asn Ile Arg Phe Gly Ile Ile Leu Arg Glu | |
| 65 70 75 | |
| GAC ATC AAA GAG CTT TTT CTT GAC CTA GCT CTC ATG TCT CAA GGC TCA | 290 |
| Asp Ile Lys Glu Leu Phe Leu Asp Leu Ala Leu Met Ser Gln Gly Ser | |
| 80 85 90 | |
| TCT GTT TTG AAT TTC TCC TAT CCC ATC TGT GAG GCG GCT CTG CCC AAG | 338 |
| Ser Val Leu Asn Phe Ser Tyr Pro Ile Cys Glu Ala Ala Leu Pro Lys | |
| 95 100 105 | |
| TTT TCT TTC TGT GGA AGA AGG AAA GGA GAG CAG ATT TAC TAT GCT GGG | 386 |
| Phe Ser Phe Cys Gly Arg Arg Lys Gly Glu Gln Ile Tyr Tyr Ala Gly | |
| 110 115 120 | |
| CCT GTC AAT AAT CCT GAA TTT ACT ATT CCT CAG GGA GAA TAC CAG GTT | 434 |
| Pro Val Asn Asn Pro Glu Phe Thr Ile Pro Gln Gly Glu Tyr Gln Val | |
| 125 130 135 140 | |
| 0 | |
| TTG CTG GAA CTG TAC ACT GAA AAA CGG TCC ACC GTG GCC TGT GCC AAT | 482 |
| Leu Leu Glu Leu Tyr Thr Glu Lys Arg Ser Thr Val Ala Cys Ala Asn | |
| 145 150 155 | |
| GCT ACT ATC ATG TGC TCC TGACTGTGGG CCTGTTAGCA AAAACTCACA | 530 |
| Ala Thr Ile Met Cys Ser | |
| 160 | |
| GCCAGCTGCA TCTCGTCGGG AACCTTCCAA GCTCCTCTGA CTGAACCTAC TGTGGGAGGA | 590 |
| GAAGCAGCTG ATGACAGAGA GAGGCTCTAC AAAGAAGCGC CCCCAAAGAG TGCAGCTGCT | 650 |
| AATTTTAGTC CCAGGACCAG ACATCCCCAG ACTCCACAGA TGTAATGAAG TCCCCGAATG | 710 |
| TATCTGTTTC TAAGGAGCCT CTTGGCAGTC CTTAAGCAGT CTTGAGGGTC CATCCTTTTT | 770 |
| CTCTAATTGG TCGCCTCCCA CCAGACTCAC CTGCTTTTCA ACTTTTTAGG AGTGCTTCCT | 830 |
| CACACGTTAC CAATAATAAA GAAAGCTGGC CACCAAAAAA AAAAAAAAAA AAAAAAAAAA | 890 |
| MKGFTATLFL WTLIFPSCSG GGGGKAWPTH VVCSDSRLEV LYQSCDPLQD FGFSVEKCSK | |
| QLKSNINIRF GIILREDIKE LFLDLALMSQ GSSVLNFSYP ICEAALPKFS FCGRRKGEQI | |
| YYAGPVNNPE FTIPQGEYQV LLELYTEKRS TVACANATIM CS | |
| primate, e.g., human MD2 (SEQ ID NO: 20 and 21) | |
| CCC CTG TTT TCT TCC ATA TTT ACT GAA GCT CAG AAG CAG TAT TGG GTC | 48 |
| Pro Leu Phe Ser Ser Ile Phe Thr Glu Ala Gln Lys Gln Tyr Trp Val | |
| 1 5 10 15 | |
| TGC AAC TCA TCC GAT GCA AGT ATT TCA TAC ACC TAC TGT GAT AAA ATG | 96 |
| Cys Asn Ser Ser Asp Ala Ser Ile Ser Tyr Thr Tyr Cys Asp Lys Met | |
| 20 25 30 | |
| CAA TAC CCA ATT TCA ATT AAT GTT AAC CCC TGT ATA G AATTGAAAGG | 143 |
| Gln Tyr Pro Ile Ser Ile Asn Val Asn Pro Cys Ile | |
| 35 40 | |
| ATCCAAAGGA TTATTGCACA TTTTCTACAT TCCAAGGAGA GATTTAAAGC AATTATATTT | 203 |
| CAATCTCTAT ATAACTGTCA ACACCATGAA TCTTCCAAAG CGCAAAGAAG TTATTTGCCG | 263 |
| AGGATCTGAT GACGATTACT CTTTTTGCAG AGCTCTGAAG GGAGAGACTG TGAATACAAC | 323 |
| AATATCATTC TCCTTCAAGG GAATAAAATT TTCTAAGGGA AAATACAAAT GTGTTGTTGA | 383 |
| AGCTATTTCT GGGAGCCCAG AAGAAATGCT CTTTTGCTTG GAGTTTGTCA TCCTACACCA | 443 |
| ACCTAATTCA AATTAGAATA AATTGAGTAT TTAAAAAAAA AAA | 486 |
| PLFSSIFTEA QKQYWVCNSS DASISYTYCD KMQYPISINV NPCI | |
| updated primate, e.g., human, MD2 (SEQ ID NO: 22 and 23): | |
| atg ttc cca ttt ctg ttt ttt tcc acc ctg ttt tct tcc ata ttt act | 48 |
| Met Phe Pro Phe Leu Phe Phe Ser Thr Leu Phe Ser Ser Ile Phe Thr | |
| 1 5 10 15 | |
| gaa gct cag aag cag tat tgg gtc tgc aac tca tcc gat gca agt att | 96 |
| Glu Ala Gln Lys Gln Tyr Trp Val Cys Asn Ser Ser Asp Ala Ser Ile | |
| 20 25 30 | |
| tca tac acc tac tgt gat aaa atg caa tac cca att tca att aat gtt | 144 |
| Ser Tyr Thr Tyr Cys Asp Lys Met Gln Tyr Pro Ile Ser Ile Asn Val | |
| 35 40 45 | |
| aac ccc tgt ata gaa ttg aaa gga tcc aaa gga tta ttg cac att ttc | 192 |
| Asn Pro Cys Ile Glu Leu Lys Gly Ser Lys Gly Leu Leu His Ile Phe | |
| 50 55 60 | |
| tac att cca agg aga gat tta aag caa tta tat ttc aat ctc tat ata 240 | |
| Tyr Ile Pro Arg Arg Asp Leu Lys Gln Leu Tyr Phe Asn Leu Tyr Ile | |
| 65 70 75 80 | |
| act gtc aac acc atg aat ctt cca aag cgc aaa gaa gtt att tgc cga | 288 |
| Thr Val Asn Thr Met Asn Leu Pro Lys Arg Lys Glu Val Ile Cys Arg | |
| 85 90 95 | |
| gga tct gat gac gat tac tct ttt tgc aga gct ctg aag gga gag act | 336 |
| Gly Ser Asp Asp Asp Tyr Ser Phe Cys Arg Ala Leu Lys Gly Glu thr | |
| 100 105 110 | |
| gtg aat aca aca ata tca ttc tcc ttc aag gga ata aaa ttt tct aag | 384 |
| Val Asn Thr Thr Ile Ser Phe Ser Phe Lys Gly Ile Lys Phe Ser Lys | |
| 115 120 125 | |
| gga aaa tac aaa tgt gtt gtt gaa gct att tct ggg agc cca gaa gaa | 432 |
| Gly Lys Tyr Lys Cys Val Val Glu Ala Ile Ser Gly Ser Pro Glu Glu | |
| 130 135 140 | |
| atg ctc ttt tgc ttg gag ttt gtc atc cta cac caa cct aat tca aat | 480 |
| Met Leu Phe Cys Leu Glu Phe Val Ile Leu His Gln Pro Asn Ser Asn | |
| 145 150 155 160 | |
| tag | 483 |
| MFPFLFFSTL FSSIFTEAQK QYWVCNSSDA SISYTYCDKM QYPISINVNP CIELKGSKGL | |
| LHIFYIPRRD LKQLYFNLYI TVNTMNLPKR KEVICRGSDD DYSFCRALKG ETVNTTISFS | |
| FKGIKFSKGK YKCVVEAISG SPEEMLFCLE FVILHQPNSN | |
| rodent, e.g., mouse, MD2 (SEQ ID NO: 24 and 25): | |
| GTCGAGTCCG ATGGTCTTC TGGCGAGTTT AAAGTATCGG AGATATTAAA TC ATG | 55 |
| Met | |
| 1 | |
| TTG CCA TTT ATT CTC TTT TCG ACG CTG CTT TCT CCC ATA TTG ACT GAA | 103 |
| Leu Pro Phe Ile Leu Phe Ser Thr Leu Leu Ser Pro Ile Leu Thr Glu | |
| 5 10 15 | |
| TCT GAG AAG CAA CAG TGG TTC TGC AAC TCC TCC GAT GCA ATT ATT TCC | 151 |
| Ser Glu Lys Gln Gln Trp Phe Cys Asn Ser Ser Asp Ala Ile Ile Ser | |
| 20 25 30 | |
| TAC AGT TAT TGT GAT GAC TTG AAA TTC CCT ATT TCA ATT AGT TCT GAA | 199 |
| Tyr Ser Tyr Cys Asp His Leu Lys Phe Pro Ile Ser Ile Ser Ser Glu | |
| 35 40 45 | |
| CCC TGC ATA AGA CTG AGG GGA ACC AAT GGA TTT GTG CAT GTT GAG TTC | 247 |
| Pro Cys Ile Arg Leu Arg Aly Thr Asn Gly Phe Val His Val Glu Phe | |
| 50 55 60 65 | |
| ATT CCA AGA GGA AAC TTA AAA TAT TTA TAT TTC AAC CTA TTC ATC AGT | 295 |
| Ile Pro Arg Gly Asn Leu Lys Tyr Leu Tyr Phe Asn Leu Phe Ile Ser | |
| 70 75 80 | |
| GTC AAC TCC ATA GAG TTG CCG AAG CGT AAG GAA GTT CTG TGC CAT GGA | 343 |
| Val Asn Ser Ile Glu Leu Pro Lys Arg Lys Glu Val Leu Cys His Gly | |
| 85 90 95 | |
| CAT GAT GAT GAC TAT TCT TTT TGC AGA GCT CTG AAA GGA GGA TAT GCT | 391 |
| His Asp Asp Asp Tyr Ser Phe Cys Arg Ala Leu Lys Gly Gly Tyr Ala | |
| 100 105 110 | |
| ATT TAGAAAATAT GAGACTGTGA ATACATCAAT ACCATTCTCT TTCGAGGGAA | 444 |
| Ile | |
| TACTATTTCC TAAGGGCCAT TACAGATGTG TTGCAGAAGC TATTGCTGGG GATA | 498 |
| MLPFILFSTL LSPILTESEK QQWFCNSSDA IISYSYCDHL KFPISISSEP CIRLRGTNGF | |
| VHVEFIPRGN LKYLYFNLFI SVNSIELPKR KEVLCHGHDD DYSFCRALKG GYAI | |
| rodent, e.g., mouse, MD2 splice variant (SEQ ID NO: 26) from deletion | |
| of nucleotides 383 through 404: | |
| MLPFILFSTLLSPILTESEKQQWFCNSSDAIISYSYCDHLKFPISISSEPCIRLRGTNGFVHVEFIPRG | |
| NLKYLYFNLFISVNSIELPKRKEVLCHGHDDDYSFCRALKGETVNTSIPFSFEGILFPKGHYRCVAEAI | |
| AGD | |

TABLE 4
Clusta1W alignment of MD protein sequenqes related to
huMD1, huMD2, and mouse MD2. See, e.g., MD1 mouse, Miyake, et al.
(1998) J. Immunol. 161:1348-1353 (SEQ ID NO: 27) is GenEank Accession
number AB007599; and MD1 Chicken, Burk, et al. (1991) EMBO J.
10:3713-3719 (SEQ ID NO: 29) is GenBank Accession number X60450
564850.

| | |
|---|---|
| MD1_HUMAN | MKGFTATLFL WTLIFPSCSG GGGGKAWPTH VVCSDSGLEV LYQSCDPLQD |
| MD1_MOUSE | MNGVAAALLV WILTSPSSSD HGSENGWPKH TACNSGGLEV VYQSCDPLQD |
| MD2_HUMAN | .......... ...PLFSSIF T...EAQKQY WVCNSSDASI SYTYCDKMQY |
| MD2_MOUSE | MLPFILFSTL LSPILT.... ....ESEKQQ WFCNSSDAII SYSYCDHLKF |
| MD1_CHICK | MKTLNVLALV LVLLCINAS. ....TEWPTH TVCKEENLEI YYKSCDPQQD |
| MD1_HUMAN | .FGFSVEKCS KQLKSNINIR FGIILREDIK ELFLDLALMS QGSSVLNFSY |
| MD1_MOUSE | .FGLSIDQCS KQIQSNLNIR FGIILRQDIR KLFLDITLMA KGSSILNYSY |
| MD2_HUMAN | PISINVNPCI ELKGSKGLLH IFYIPRRDLK QLYFNLYITV NTMNLPKRKE |
| MD2_MOUSE | PISISSEPCI RLRGTNGFVH VEFIPRGNLK YLYFNLFISV NSIELPKRKE |
| MD1_CHICK | .FAFSIDRCS DVTTHTFDIR AAMVLRQSIK ELYAKVDLII NGKTVLSYSE |
| MD1_HUMAN | PICEAALPKF SFCGRRKGEQ IYYAGPVNNP EFTIPQGEYQ VLLELYT.EK |
| MD1_MOUSE | PLCEEDQPKF SFCGRRKGEQ IYYAGPVNNP GLDVPQGEYQ LLLELYN.EN |
| MD2_HUMAN | VICRGSDDDY SFCRALKGET VNTTISFSFK GIKFSKGKYK CVVEAISGSP |
| MD2_MOUSE | VLCHGHDDDY SFCRALKGET VNTSIPFSFE GILFPKGHYR CVAEAIAGD- |
| MD1_CHICK | TLCGPGLSKL IFCGKKKGEH LYYEGPITLG IKEIPQGDYT ITARLTN.ED |
| MD1_HUMAN | RSTVACANAT IMCS.... |
| MD1_MOUSE | RATVACANAT VTSS.... |
| MD2_HUMAN | EEMLFCLEFV ILHQPNSN |
| MD2_MOUSE | ---------- -------- |
| MD1_CHICK | RATVACADFT VKNYLDY. |

The chemokine protein of this invention is defined in part by its physicochemical and biological properties. The biological properties of the chemokine described herein, e.g., HCC5, is defined, in part, by its amino acid sequence, and mature size, and various structural features conserved across chemokines. See, e.g., Wells (1997) J. Leuk. Biol. 61:545-550. The HCC5 chemokine is most closely related in sequence to the chemokines, Human chemokine HCCl; Pituitary expressed chemokine (PGEC); Human MIP-4 (a chemoattractant for leukocytes); Macrophage inflammatory protein-1-gamma (MIP-1γ); and Stem cell mobilizing chemokine (CKbeta-1).

The proteins sharing motifs characteristic of deubiquitinating proteins of the invention described herein, e.g., Dub11 and Dub12, are also defined in part, by their physicochemical, biological, and structural properties. The biological properties of the chemokine described herein, e.g., Dub11 and Dub12, are defined, in part, by their amino acid sequences, and mature size, and various structural features conserved across other deubiquitinating proteins, e.g., Dub1 and Dub2. See, e.g., Zhu, et al. (1996) Proc. Natl. Acad. Sci. USA 93:3275-3279 (SEQ ID NO: 16; GenBank Accession number U41636); and Zhu, et al. (1997) J. Biol. Chem. 272:51-57 (SEQ ID NO: 17; GenBank Accession number U70368).

The MD proteins of the invention were discovered through searches and careful analysis of database sequences, e.g., MD1 and MD2 are also defined in part by their physicochemical, biological, and structural properties. The biological properties of these LLR-like proteins described herein, e.g., primate MD1, primate MD2, and rodent MD2 are defined, in part, by their amino acid sequence, mature size, and various structural features conserved across other MD proteins, e.g., chicken MD and mouse MD. See, e.g., MD1 mouse, Miyake, et al. (1998) J. Immunol. 161:1348-1353 (SEQ ID NO: 27; GenBank Accession number AB007599); and MD1 Chicken, Burk, et al. (1991) EMBO J. 10:3713-3719 (SEQ ID NO: 29; GenBank Accession number X60450 S64850).

One of ordinary skill will readily recognize that some sequence variations may be tolerated in the proteins described herein, e.g., conservative substitutions or positions remote from the critical residues for receptor interaction or important tertiary structure features, without altering significantly the biological activity of the molecule. Antigenic activity will be retained where epitopes on the protein are conserved. Conversely, non-conservative substitutions may be adapted to delete selected functions or activities. Some predictability on biological effect of changes may be derived from comparisons among species and allelic variations in sequences of these and related chemokines.

It is possible that the HCC5 may actually be an antagonist of one, some, or all, of many related chemokines. In such case, combination compositions may be desired. For example, a combined group of functional agonists and antagonists for specific receptors may be called for, e.g., a combination of chemokines and antibody antagonists of others. In addition, HCC5 may be useful to block HIV or HTLV infection, which viruses may use the respective receptors for infection.

The HCC5 chemokine is seemingly specifically expressed, since its sequence has not appeared from many sources. The structural similarity to other chemokines suggests that signals important in inflammation, cell differentiation, and development are mediated by it. See, e.g., Gilbert (1991) Developmental Biology (3d ed.) Sinauer Associates, Sunderland, Mass.; Browder, et al. (1991) Developmental Biology (3d ed.) Saunders, Philadelphia, Pa.; Russo, et al. (1992) Development: The Molecular Genetic Approach Springer-Verlag, New York, N.Y.; and Wilkins (1993) Genetic Analysis of Animal Development (2d.ed.) Wiley-Liss, New York, N.Y. Moreover, HCC5 expression or responsiveness should serve as a marker, e.g., to define certain cell subpopulations.

The HCC5 chemokine was discovered through searches and careful analysis of database sequences. The HCC5 sequence was discovered in a cDNA library from pooled bulk breast tumor tissue. Absence of overlapping sequences from other sources suggests extremely specific tissue expression, or highly regulated expression. Amino acid homology analysis suggests that the HCC5 gene encodes a member of a group of related family of chemokines. See, e.g., human stem cell mobilizing chemokine (CKbeta-1): Kreider, et al. (1997) Patent WO 9715594 (SEQ ID NO: 3) is GenBank Accession number 97P-W17659; macrophage inflammatory protein-1-gamma (MIP-1): Adams, et al. (1995) Patent WO 9517092 (SEQ ID NO: 4) is GenBank Accession number 95P-R76128; human MIP-4: a chemoattractant for leukocytes; Adams, et al. (1997) Patent WO 9634891 (SEQ ID NO: 5) is GenBank Accession number 96P-WO7203; pituitary expressed chemokine (PGEC): Bandman, et al. Patent WO 9616979 (SEQ ID NO: 6) is GenBank Accession number 96P-R95691; and human chemokine HCC1: Forsmann, et al. (1998) Patent WO 9741230 (SEQ ID NO: 7) is GenBank Accession number 97P-W38171.

Northern blot analysis can be performed using standard methods, see, e.g., Maniatis, et al. (1982) Molecular Cloning: A Laboratory Manual Cold Spring Harbor Laboratory, Cold Spring Harbor Press, NY; Sambrook, et al. (1989) Molecular Cloning: A Laboratory Manual (2d ed.) Vols. 1-3, CSH Press, NY; Ausubel, et al., Bioloay Greene Publishing Associates, Brooklyn, NY; and Ausubel, et al. (1987 and Supplements) Current Protocols in Molecular Biology Wiley/Greene, NY. See also below.

II. Definitions

The term “binding composition” refers to molecules that bind with affinity and selectivity to the HCC5 chemokine, e.g., in an antibody-antigen interaction. However, other compounds, e.g., receptor proteins, may also specifically and/or selectively associate with HCC5 to the exclusion of other molecules. Typically, the association will be in a natural physiologically relevant protein-protein interaction, either covalent or non-covalent, and may include members of a multiprotein complex, including carrier compounds or dimerization partners. The molecule may be a polymer, or chemical reagent. No implication as to whether a HCC5 chemokine is necessarily a convex shaped molecule, e.g., the ligand or the receptor of a ligand-receptor interaction, is necessarily represented, other than whether the interaction exhibits similar specificity, e.g., specific affinity. A functional analog may be a ligand with structural modifications, e.g., a mutein, or may be a wholly unrelated molecule, e.g., which has a molecular shape which interacts with the appropriate ligand binding determinants. The ligands may serve as agonists or antagonists of the receptor, see, e.g., Goodman, et al. Goodman & Gilman's: The Pharmacological Bases of Therapeutics (current edition) Pergamon Press, Tarrytown, N.Y.

The term “binding agent:antigen complex”, as used herein, refers to a complex of a binding agent and antigen, e.g., an HCC5 chemokine protein that is formed by specific binding of the binding agent to, e.g., the HCC5 chemokine protein. Specific or selective binding of the binding agent means that the binding agent has a specific binding site, e.g., antigen binding site, that recognizes a site on the HCC5 chemokine protein. For example, antibodies raised to a HCC5 chemokine protein and recognizing an epitope on the chemokine protein are capable of forming a binding agent:HCC5 chemokine protein complex by specific binding. Typically, the formation of a binding agent:HCC5 chemokine protein complex allows the measurement of HCC5 chemokine protein in a mixture of other proteins and biologics. The term “antibody:HCC5 chemokine protein complex” refers to an embodiment in which the binding agent, e.g., is the antigen binding portion from an antibody. The antibody may be monoclonal, polyclonal, or a binding fragment of an antibody, e.g., an Fab or F(ab)2 fragment. The antibody will preferably be a polyclonal antibody for cross-reactivity testing purposes.

“Homologous” nucleic acid sequences, when compared, exhibit significant similarity, or identity. The standards for homology in nucleic acids are either measures for homology generally used in the art by sequence comparison and/or phylogenetic relationship, or based upon hybridization conditions. Hybridization conditions are described in greater detail below.

An “isolated” nucleic acid is a nucleic acid, e.g., an RNA, DNA, or a mixed polymer, which is substantially separated from other biologic components which naturally accompany a native sequence, e.g., proteins and flanking genomic sequences from the originating species. The term embraces a nucleic acid sequence which has been removed from its naturally occurring environment, and includes recombinant or cloned DNA isolates and chemically synthesized analogs, or analogs biologically synthesized by heterologous systems. A substantially pure molecule includes isolated forms of the molecule. Alternatively, a purified species may be separated from host components from a recombinant expression system. Generally, the nucleic acid will be in a vector or fragment less than about 50 kb, usually less than about 30 kb, typically less than about 10 kb, and preferably less than about 6 kb.

An isolated nucleic acid will usually contain homogeneous nucleic acid molecules, but will, in some embodiments, contain nucleic acids with minor sequence heterogeneity. This heterogeneity is typically found at the polymer ends or portions not critical to a desired biological function or activity.

As used herein, the term “HCC5 chemokine protein,” shall encompass, when used in a protein context, a protein having amino acid sequence, particularly from the chemokine motif portion, shown in Table 1, SEQ ID NO: 2, or a significant fragment characteristic of such a protein, preferably a natural embodiment. The invention also embraces a polypeptide which exhibits similar structure to HCC5 chemokine, e.g., which interacts with HCC5 chemokine specific binding components. These binding components, e.g., antibodies, typically bind to a HCC5 chemokine with high affinity, e.g., at least about 100 nM, usually better than about 30 nM, preferably better than about 10 nM, and more preferably at better than about 3 nM.

As used herein, the term “Dub11 protein,” or “Dub12 protein,” shall encompass, when used in a protein context, a protein having amino acid sequences, particularly from Table 2, e.g., SEQ ID NO: 9 or 11, or 13 or 15, or a significant fragment characteristic of such a protein, preferably a natural embodiment. The invention also embraces a polypeptide which exhibits similar structure to “Dub11 protein” or “Dub12 protein”, e.g., which interacts with “Dub11 protein,” or “Dub12 protein” specific binding components. These binding components, e.g., antibodies, typically bind to a “Dub11 protein” or “Dub12 protein” with high affinity, e.g., at least about 100 nM, usually better than about 30 nM, preferably better than about 10 nM, and more preferably at better than about 3 nM. Still higher affinities are possible, e.g., 100 pM, 30 pM, 100 fM, etc.

As used herein, the terms “primate MD1 protein,” “primate MD2 protein,” or “rodent MD2 protein,” shall encompass, when used in a protein context, a protein having amino acid sequences, e.g., from Table 3, particularly the coding portions of SEQ ID NO: 19; 21 or 23; or 25 or 26, or a significant fragment characteristic of such a protein, preferably a natural embodiment. The invention also embraces a polypeptide which exhibits similar structure to “primate MD1 protein,” “primate MD2 protein,” or “rodent MD2 protein,”, e.g., which interacts with “primate MD1 protein,” “primate MD2 protein,” or “rodent MD2 protein,” specific binding components. These binding components, e.g., antibodies, typically bind to a “primate MD1 protein,” “primate MD2 protein,” or “rodent MD2 protein,” with high affinity, e.g., at least about 100 nM, usually better than about 30 nM, preferably better than about 10 nM, and more preferably at better than about 3 nM.

The term “polypeptide” or “protein” as used herein includes a significant fragment or segment or chemokine motif portion of, e.g., a HCC5 chemokine; a significant fragment or segment of the deubiquitinating motif portion of, e.g., Dub11 or Dub12; a significant fragment or segment or the LRR motif portion of, e.g., primate MD1, primate MD2, or rodent MD2; and encompasses a stretch of amino acid residues of at least about 8 amino acids, generally at least 10 amino acids, more generally at least 12 amino acids, often at least 14 amino acids, more often at least 16 amino acids, typically at least 18 amino acids, more typically at least 20 amino acids, usually at least 22 amino acids, more usually at least 24 amino acids, preferably at least 26 amino acids, more preferably at least 28 amino acids, and, in particularly preferred embodiments, at least about 30 or more amino acids, e.g., 35, 40, 45, 50, 60, 70, etc. The segments may have amino and carboxy termini, with appropriate lengths, e.g., starting at residue 1, 2, 3, etc., and ending at the carboxy-terminus, e.g., residue 61, 60, 59, 58, etc. Preferred fragments include those running from, e.g., 1-24; 24-40; 40-61; or intact structural segments (such as helical or beta sheet segments, see PHD (Rost and Sander (1994) Proteins 19:55-72) and DSC (King and Sternberg (1996) Protein Sci. 5:2298-2310) structural analysis software). The invention encompasses proteins comprising a plurality of the segments.

A “recombinant” nucleic acid is defined either by its method of production or its structure. In reference to its method of production, e.g., a product made by a process, the process is use of recombinant nucleic acid techniques, e.g., involving human intervention in the nucleotide sequence, typically selection or production. Alternatively, it can be a nucleic acid made by generating a sequence comprising fusion of two fragments which are not naturally contiguous to each other, but is meant to exclude products of nature, e.g., naturally occurring mutants. These encompass, for example, products made by transforming cells with a non-naturally occurring vector, constructs producing operable association of the gene with a heterologous promoter, and nucleic acids comprising sequence derived using a synthetic oligonucleotide process. Such is often done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the commonly available natural forms. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., fusion, polypeptide. Specifically included are synthetic nucleic acids which, by genetic code redundancy, encode polypeptides similar to fragments of these antigens, and fusions of sequences from various different species variants.

“Solubility” is reflected by sedimentation measured in Svedberg units, which are a measure of the sedimentation velocity of a molecule under particular conditions. The determination of the sedimentation velocity was classically performed in an analytical ultracentrifuge, but is typically now performed in a standard ultracentrifuge. See, Freifelder (1982) Physical Biochemistry (2d ed.) W.H. Freeman & Co., San Francisco, Calif.; and Cantor and Schimmel (1980) Biophysical Chemistry parts 1-3, W.H. Freeman & Co., San Francisco, Calif. As a crude determination, a sample containing a putatively soluble polypeptide is spun in a standard full sized ultracentrifuge at about 50K rpm for about 10 minutes, and soluble molecules will remain in the supernatant. A soluble particle or polypeptide will typically be less than about 30S, more typically less than about 15S, usually less than about 10S, more usually less than about 6S, and, in particular embodiments, preferably less than about 4S, and more preferably less than about 3S. Solubility of a polypeptide or fragment depends upon the environment and the polypeptide. Many parameters affect polypeptide solubility, including temperature, electrolyte environment, size and molecular characteristics of the polypeptide, and nature of the solvent. Typically, the temperature at which the polypeptide is used ranges from about 4° C. to about 65° C. Usually the temperature at use is greater than about 18° C. and more usually greater than about 22° C. For diagnostic purposes, the temperature will usually be about room temperature or warmer, but less than the denaturation temperature of components in the assay. For therapeutic purposes, the temperature will usually be body temperature, typically about 37° C. for humans, though under certain situations the temperature may be raised or lowered in situ or in vitro.

The size and structure of the polypeptide should generally be in a substantially stable state, and usually not in a denatured state. The polypeptide may be associated with other polypeptides in a quaternary structure, e.g., to confer solubility, or associated with lipids or detergents in a manner which approximates natural lipid bilayer interactions.

The solvent will usually be a biologically compatible buffer, of a type used for preservation of biological activities, and will usually approximate a physiological solvent. Usually the solvent will have a neutral pH, typically between about 5 and 10, and preferably about 7.5. On some occasions, a detergent will be added, typically a mild non-denaturing one, e.g., CHS (cholesterylhemisuccinate) or CHAPS (3-[3-cholamidopropyl-dimethylammonio]-1-propane sulfonate), or a low enough concentration as to avoid significant disruption of structural or physiological properties of the protein.

“Substantially pure” in a protein context typically means that the protein is isolated from other contaminating proteins, nucleic acids, and other biologicals derived from the original source organism. Purity, or “isolation” may be assayed by standard methods, and will ordinarily be at least about 50% pure, more ordinarily at least about 60% pure, generally at least about 70% pure, more generally at least about 80% pure, often at least about 85% pure, more often at least about 90% pure, preferably at least about 95% pure, more preferably at least about 98% pure, and in most preferred embodiments, at least 99% pure. Similar concepts apply, e.g., to antibodies or nucleic acids.

“Substantial similarity” in the nucleic acid sequence comparison context means either that the segments, or their complementary strands, when compared, are identical when optimally aligned, with appropriate nucleotide insertions or deletions, in at least about 50% of the nucleotides, generally at least 56%, more generally at least 59%, ordinarily at least 62%, more ordinarily at least 65%, often at least 68%, more often at least 71%, typically at least 74%, more typically at least 77%, usually at least 80%, more usually at least about 85%, preferably at least about 90%, more preferably at least about 95 to 98% or more, and in particular embodiments, as high at about 99% or more of the nucleotides. Alternatively, substantial similarity exists when the segments will hybridize under selective hybridization conditions, to a strand, or its complement, typically using a sequence derived, e.g., from SEQ ID NO: 1. Typically, selective hybridization will occur when there is at least about 55% similarity over a stretch of at least about 30 nucleotides, preferably at least about 65% over a stretch of at least about 25 nucleotides, more preferably at least about 75%, and most preferably at least about 90% over about 20 nucleotides. See Kanehisa (1984) Nuc. Acids Res. 12:203-213. The length of similarity comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides, e.g., 150, 200, etc.

The invention also provides compositions which exhibit a plurality of distinct, e.g., nonoverlapping, segments of the specified length. Typically, the plurality will be at least two, more usually at least three, and preferably 5, 7, or even more. While the length minima are provided, longer lengths, of various sizes, may be appropriate, e.g., one of length 7, and two of length 12.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optical alignment of sequences for comparison can be conducted, e.g., by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Nat'l Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.); the NCBI, run by the National Institutes of Health; or by visual inspection (see generally Ausubel, et al., supra).

One example of a useful algorithm is PILEUP. PILEUP creates a multiple sequence alignment from a group of related sequences using progressive, pairwise alignments to show relationship and percent sequence identity. It also plots a tree or dendrogram showing the clustering relationships used to create the alignment. PILEUP uses a simplification of the progressive alignment method of Feng and Doolittle (1987) J. Mol. Evol. 35:351-360. The method used is similar to the method described by Higgins and Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described Altschul, et al. (1990) J. Mol. Biol. 215:403-410. Software for performing BLAST analyses is publicly available through the National Center for Biotechnology Information (http:www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff (1989) Proc. Nat'l Acad. Sci. USA 89:10915) alignments (B) of 50, expectation (E) of 10, M=5, N=4, and a comparison of both strands.

In addition to calculating percent sequence identity, the BLAST algorithm also performs a statistical analysis of the similarity between two sequences (see, e.g., Karlin and Altschul (1993) Proc. Nat'l Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences of polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

“Stringent conditions”, in referring to homology or substantial similarity in the hybridization context, will be stringent combined conditions of salt, temperature, organic solvents, and other parameters, typically those controlled in hybridization reactions. The combination of parameters is more important than the measure of any single parameter. See, e.g., Wetmur and Davidson (1968) J. Mol. Biol. 31:349-370. A nucleic acid probe which binds to a target nucleic acid under stringent conditions is specific for the target nucleic acid. Such a probe is typically more than 11 nucleotides in length, and is sufficiently identical or complementary to a target nucleic acid over the region specified by the sequence of the probe to bind the target under stringent hybridization conditions. Preferably, the available coding segment will hybridize to natural message under wash conditions of about 55° C., or 60° C. or 65° C.; and low salt, e.g., 200 mM. Hybridization under stringent conditions should give a background of at least 2-fold over background, preferably at least 3-5 or more.

HCC5 chemokines; Dub11 or Dub12 proteins; and MD1 or MD2 proteins from other mammalian species can be cloned and isolated by cross-species hybridization of closely related species. Similarity may be relatively low between distantly related species, and thus hybridization of relatively closely related species is advisable. Alternatively, preparation of an antibody preparation which exhibits less species specificity may be useful in expression cloning approaches.

The phrase “specifically binds to an antibody” or “specifically immunoreactive with”, when referring to a protein or peptide, refers to a binding reaction which is determinative of the presence of the protein in the presence of a heterogeneous population of proteins and other biological components. Thus, under designated immunoassay conditions, the specified antibodies bind to a particular protein and do not significantly bind other proteins present in the sample. Specific binding to an antibody under such conditions may require an antibody that is selected for its specificity or selectivity for a particular protein. For example, antibodies raised to the HCC5 chemokines; Dub11 or Dub12 proteins; and MD1 or MD2 protein's immunogen with the amino acid sequence depicted in, respectively, SEQ ID NO: 2; 9 or 11; 13 or 15; 21 or 23; 25 or 26, can be selected to obtain antibodies specifically immunoreactive with HCC5 chemokines; Dub11 or Dub12 proteins; and MD1 or MD2 proteins and not with other proteins. The antibodies may be species specific, e.g., also recognizing polymorphic and splicing or developmental variants.

A significant “fragment” in a nucleic acid context is a contiguous segment of at least about 17 nucleotides, generally at least about 22 nucleotides, ordinarily at least about 29 nucleotides, more often at least about 35 nucleotides, typically at least about 41 nucleotides, usually at least about 47 nucleotides, preferably at least about 55 nucleotides, and in particularly preferred embodiments will be at least about 60 or more nucleotides. Analogous concepts apply to polypeptides.

III. Nucleic Acids

Nucleic acids which encodes the described proteins, or fragments thereof, can be obtained by chemical synthesis, screening cDNA libraries, or screening genomic libraries prepared from a wide variety of cell lines or tissue samples. See, e.g., Okayama and Berg (1982) Mol. Cell. Biol. 2:161-170; Gubler and Hoffman (1983) Gene 25:263-269; and Glover (ed. 1984) DNA Cloning: A Practical Approach, IRL Press, Oxford. Alternatively, the sequences provided herein provide useful PCR primers or allow synthetic or other preparation of suitable genes encoding a receptor; including, naturally occurring embodiments.

DNA can be expressed in a wide variety of host cells for the synthesis of a full-length protein, or fragments, which can in turn, e.g., be used to generate polyclonal or monoclonal antibodies; for binding studies; for construction and expression of modified molecules; for structure/function studies; and for controls in detection assays. Each antigen or its fragments can be expressed in host cells that are transformed or transfected with appropriate expression vectors. These molecules can be substantially purified to be free of protein or cellular contaminants, other than those derived from the recombinant host, and therefore are particularly useful in pharmaceutical compositions when combined with a pharmaceutically acceptable carrier and/or diluent. The antigen, or portions thereof, may be expressed as fusions with other proteins.

HCC5 chemokines; Dub11 or Dub12 proteins; and MD1 or MD2 proteins are exemplary of structurally and functionally related proteins. The soluble HCC5 chemokine protein will serve to transmit signals between different cell types. The preferred embodiments of HCC5 chemokines; Dub11 or Dub12 proteins; and MD1 or MD2 proteins as disclosed, will be useful in standard procedures to isolate genes from different individuals or other species, e.g., warm blooded animals, such as birds and mammals. Cross hybridization will allow isolation of related genes encoding proteins from individuals, strains, or species. They will be useful for isolating genes from domestic pets, e.g., dogs and cats, and livestock, e.g., horse, pigs, cattle, sheep, chickens, turkeys, fish, etc. A number of different approaches are available to successfully isolate a suitable nucleic acid clone based upon the information provided herein. Southern blot hybridization studies can qualitatively determine the presence of homologous genes in human, monkey, rat, mouse, dog, cat, cow, and rabbit genomes under specific hybridization conditions.

Complementary sequences will also be used as probes or primers. Based upon identification of the likely amino terminus, other peptides should be particularly useful, e.g., coupled with anchored vector or poly-A complementary PCR techniques or with complementary DNA of other peptides.

Techniques for nucleic acid manipulation of genes encoding HCC5 chemokines; Dub11 or Dub12 proteins; and MD1 or MD2 proteins, such as subcloning nucleic acid sequences encoding polypeptides into expression vectors, labeling probes, DNA hybridization, and the like are described generally in Sambrook, et al. (1989) Molecular Cloning: A Laboratory Manual (2nd ed.) Vol. 1-3, Cold Spring Harbor Laboratory, Cold Spring Harbor Press, NY, which is incorporated herein by reference. This reference manual is hereinafter referred to as “Sambrook, et al.”

There are various methods of isolating DNA sequences encoding HCC5 chemokines; Dub11 or Dub12 proteins; and MD1 or MD2 proteins. For example, DNA is isolated from a genomic or cDNA library using labeled oligonucleotide probes having sequences identical or complementary to portions or all of the sequences disclosed herein. Full-length probes may be used, or oligonucleotide probes may be generated by comparison of the sequences disclosed. Such probes can be used directly in hybridization assays to isolate DNA encoding HCC5 chemokines; Dub11 or Dub12 proteins; and MD1 or MD2 proteins, or probes can be designed for use in amplification techniques such as PCR, for the isolation of DNA encoding HCC5 chemokines; Dub11 or Dub12 proteins; and MD1 or MD2 proteins. Reverse translation computer programs can also provide alternative nucleic acid sequences which encode the same proteins. Methods for extension of partial sequence are readily available, and well known in the art.

To prepare a cDNA library, mRNA is isolated from cells which express a HCC5 chemokine; a Dub11 or Dub12 protein; or an MD1 or MD2 protein. cDNA is prepared from the mRNA and ligated into a recombinant vector. The vector is transfected into a recombinant host for propagation, screening, and cloning. Methods for making and screening cDNA libraries are well known. See Gubler and Hoffman (1983) Gene 25:263-269 and Sambrook, et al.

For a genomic library, the DNA can be extracted from tissue and either mechanically sheared or enzymatically digested to yield fragments of about 12-20 kb. The fragments are then separated by gradient centrifugation and cloned in bacteriophage lambda vectors. These vectors and phage are packaged in vitro, as described in Sambrook, et al. Recombinant phage are analyzed by plaque hybridization as described in Benton and Davis (1977) Science 196:180-182. Colony hybridization is carried out as generally described in e.g., Grunstein, et al. (1975) Proc. Nat'l Acad. Sci. USA. 72:3961-3965.

DNA encoding an HCC5 chemokines; Dub11 or Dub12 proteins; and MD1 or MD2 proteins can be identified in either cDNA or genomic libraries by its ability to hybridize with the nucleic acid probes described herein, e.g., in colony or plaque hybridization assays. The corresponding DNA regions are isolated by standard methods familiar to those of skill in the art. See, e.g., Sambrook, et al.

Various methods of amplifying target sequences, such as the polymerase chain reaction, can also be used to prepare DNA encoding an HCC5 chemokine; a Dub11 or Dub12 protein; or an MD1 or MD2 protein. Polymerase chain reaction (PCR) technology is used to amplify such nucleic acid sequences directly from mRNA, from cDNA, and from genomic libraries or cDNA libraries. The isolated sequences encoding an HCC5 chemokine; a Dub11 or Dub12 protein; or an MD1 or MD2 protein may also be used as templates for PCR amplification.

Typically, in PCR techniques, oligonucleotide primers from two 5′ regions flanking the DNA region to be amplified are synthesized. The polymerase chain reaction is then carried out using the two primers. See Innis, et al. (eds. 1990) PCR Protocols: A Guide to Methods and Applications Academic Press, San Diego, Calif. Primers can be selected to amplify the entire regions encoding a full-length HCC5 chemokine protein or to amplify smaller DNA segments as desired. Once such regions are PCR-amplified, they can be sequenced and oligonucleotide probes can be prepared from sequence obtained using standard techniques. These probes can then be used to isolate DNAs encoding an HCC5 chemokine; a Dub11 or Dub12 protein; or an MD1 or MD2 protein.

Oligonucleotides for use as probes are usually chemically synthesized according to the solid phase phosphoramidite triester method first described by Beaucage and Carruthers (1983) Tetrahedron Lett. 22(20):1859-1862, or using an automated synthesizer, as described in Needham-VanDevanter, et al. (1984) Nucleic Acids Res. 12:6159-6168. Purification of oligonucleotides is performed, e.g., by native acrylamide gel electrophoresis or by anion-exchange HPLC as described in Pearson and Regnier (1983) J. Chrom. 255:137-149. The sequence of the synthetic oligonucleotide can be verified using, e.g., the chemical degradation method of Maxam and Gilbert in Grossman and Moldave (eds. 1980) Methods in Enzymology 65:499-560 Academic Press, New York.

An isolated nucleic acid encoding a portion of an HCC5 chemokine; a Dub11 or Dub12 protein; or an MD1 or MD2 protein was identified. The nucleotide sequence and corresponding open reading frame are provided in SEQ ID NO: 1; 8 or 10; 12 or 14; 20 or 22; 22; or 24. The invention provides compositions which exhibit a plurality of distinct, e.g., nonoverlapping, segments of the specified length. Typically, the plurality will be at least two, more usually at least three, and preferably 5, 7, or even more. While the length minima are provided, longer lengths, of various sizes, may be appropriate, e.g., one of length 7, and two of length 12.

The HCC5 chemokine exhibits limited similarity to portions of known chemokines. See, e.g., Matsushima and Oppenheim (1989) Cytokine 1:2-13; Oppenheim, et al. (1991) Ann. Rev. Immunol. 9:617-648; Schall (1991) Cytokine 3:165-183; and Gronenborn and Clore (1991) Protein Engineering 4:263-269. Other features of comparison are apparent between the HCC5 chemokine and chemokine families. See, e.g., Lodi, et al. (1994) Science 263:1762-1766. In particular, β-sheet and α-helix residues can be determined using, e.g., RASMOL program, see Sayle and Milner-White (1995) TIBS 20:374-376; or Gronenberg, et al. (1991) Protein Engineering 4:263-269; and other structural features are defined in Lodi, et al. (1994) Science 263:1762-1767. These secondary and tertiary features assist in defining further the C, CC, CXC, and CX3C structural features, along with spacing of appropriate cysteine residues.

This invention provides isolated DNA or fragments to encode an HCC5 chemokine; a Dub11 or Dub12 protein; or an MD1 or MD2 protein. In addition, this invention provides isolated or recombinant DNA which encodes a protein or polypeptide which is capable of hybridizing under appropriate conditions, e.g., high stringency, with the DNA sequences described herein. The biologically active protein or polypeptide can be an intact ligand, or fragment, and have an amino acid sequence as disclosed in SEQ ID NO: 2; 9 or 11; 13 or 15; 21 or 23; 25 or 26, particularly natural embodiments. Preferred embodiments will be full length natural sequences, or fragments of at least about 6,000 daltons, more preferably at least about 8,000 daltons. Further, this invention contemplates the use of isolated or recombinant DNA, or fragments thereof, which encode proteins which are homologous to an HCC5 chemokine; a Dub11 or Dub12 protein; or an MD1 or MD2 protein or which were isolated using cDNA encoding an HCC5 chemokine; a Dub11 or Dub12 protein; or an MD1 or MD2 protein. The isolated DNA can have the respective regulatory sequences in the 5′ and 3′ flanks, e.g., promoters, enhancers, poly-A addition signals, and others. Also embraced are methods for making expression vectors with these sequences, or for making, e.g., expressing and purifying, protein products.

Recombinant clones derived from the genomic sequences, e.g., containing introns, will be useful for transgenic studies, including, e.g., transgenic cells and organisms, and for gene therapy. See, e.g., Goodnow (1992) “Transgenic Animals” in Roitt (ed.) Encyclopedia of Immunology, Academic Press, San Diego, pp. 1502-1504; Travis (1992) Science 256:1392-1394; Kuhn, et al. (1991) Science 254:707-710; Capecchi (1989) Science 244:1288; Robertson (1987 ed.) Teratocarcinomas and Embryonic Stem Cells: A Practical Approach, IRL Press, Oxford; and Rosenberg (1992) J. Clinical Oncology 10:180-199.

IV. Making Protein

DNAs which encode an HCC5 chemokine; a Dub11 or Dub12 protein; or an MD1 or MD2 protein or fragments thereof can be obtained by chemical synthesis, screening cDNA libraries, or by screening genomic libraries prepared from a wide variety of cell lines or tissue samples. Methods for doing so, or making expression vectors, are described herein.

These DNAs can be expressed in a wide variety of host cells for the synthesis of a full-length protein or fragments which can in turn, e.g., be used to generate polyclonal or monoclonal antibodies; for binding studies; for construction and expression of modified molecules; and for structure/function studies. Each HCC5 chemokine; Dub11 or Dub12 protein; or MD1 or MD2 protein or its fragments can be expressed in host cells that are transformed or transfected with appropriate expression vectors. These molecules can be substantially purified to be free of protein or cellular contaminants, other than those derived from the recombinant host, and therefore are particularly useful in pharmaceutical compositions when combined with a pharmaceutically acceptable carrier and/or diluent. The antigen, e.g., an HCC5 chemokine; a Dub11 or Dub12 protein; or an MD1 or MD2 protein, or portions thereof, may be expressed as fusions with other proteins or possessing an epitope tag.

Expression vectors are typically self-replicating DNA or RNA constructs containing the desired antigen gene or its fragments, usually operably linked to appropriate genetic control elements that are recognized in a suitable host cell. The specific type of control elements necessary to effect expression will depend upon the eventual host cell used. Generally, the genetic control elements can include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. Expression vectors also usually contain an origin of replication that allows the vector to replicate independently from the host cell.

The vectors of this invention contain DNAs which encode an HCC5 chemokine; a Dub11 or Dub12 protein; or an MD1 or MD2 protein, or a fragment thereof, typically encoding, e.g., a biologically active or immunogenic polypeptide, or protein. The DNA can be under the control of a viral promoter and can encode a selection marker. This invention further contemplates use of such expression vectors which are capable of expressing eukaryotic cDNA coding for an HCC5 chemokine; a Dub11 or Dub12 polypeptide; or an MD1 or MD2 polypeptide in a prokaryotic or eukaryotic host, where the vector is compatible with the host and where the eukaryotic cDNA coding for the protein is inserted into the vector such that growth of the host containing the vector expresses the cDNA in question. Usually, expression vectors are designed for stable replication in their host cells or for amplification to greatly increase the total number of copies of the desirable gene per cell. It is not required that an expression vector replicate in a host cell, e.g., it is possible to effect transient expression of the protein or its fragments in various hosts using vectors that do not contain a replication origin that is recognized by the host cell. It is also possible to use vectors that cause integration of an HCC5 chemokine gene; a Dub11 or Dub12 gene; or an MD1 or MD2 gene or its fragments into the host DNA by recombination, or to integrate a promoter which controls expression of an endogenous gene.

Vectors, as used herein, contemplate plasmids, viruses, bacteriophage, integratable DNA fragments, and other vehicles which enable the integration of DNA fragments into the genome of the host. Expression vectors are specialized vectors which contain genetic control elements that effect expression of operably linked genes. Plasmids are the most commonly used form of vector, but many other forms of vectors which serve an equivalent function are suitable for use herein. See, e.g., Pouwels, et al. (1985 and Supplements) Cloning Vectors: A Laboratory Manual Elsevier, N.Y.; and Rodriguez, et al. (eds. 1988) Vectors: A Survey of Molecular Cloning Vectors and Their Uses Buttersworth, Boston, Mass.

For purposes of this invention, DNA sequences are operably linked when they are functionally related to each other. For example, DNA for a presequence or secretory leader is operably linked to a polypeptide if it is expressed as a preprotein or participates in directing the polypeptide to the cell membrane or in secretion of the polypeptide. A promoter is operably linked to a coding sequence if it controls the transcription of the polypeptide; a ribosome binding site is operably linked to a coding sequence if it is positioned to permit translation. Usually, operably linked means contiguous and in reading frame, however, certain genetic elements such as repressor genes are not contiguously linked but still bind to operator sequences that in turn control expression. See e.g., Rodriguez, et al., Chapter 10, pp. 205-236; Balbas and Bolivar (1990) Methods in Enzymol. 185:14-37; and Ausubel, et al. (1993) Current Protocols in Molecular Biology, Greene and Wiley, NY.

Suitable host cells include prokaryotes, lower eukaryotes, and higher eukaryotes. Prokaryotes include both gram negative and gram positive organisms, e.g., E. coli and B. subtilis. Lower eukaryotes include yeasts, e.g., S. cerevisiae and Pichia, and species of the genus Dictyostelium. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. As used herein, E. coli and its vectors will be used generically to include equivalent vectors used in other prokaryotes. A representative vector for amplifying DNA is pBR322 or its derivatives. Vectors that can be used to express HCC5 chemokines; Dub11 or Dub12 proteins; and MD1 or MD2 proteins or HCC5 chemokines; Dub11 or Dub12 protein; and MD1 or MD2 protein fragments include, but are not limited to, such vectors as those containing the lac promoter (pUC-series); trp promoter (pBR322-trp); Ipp promoter (the pIN-series); lambda-pP or pR promoters (pOTS); or hybrid promoters such as ptac (pDR540). See, e.g., Brosius, et al. (1988) “Expression Vectors Employing Lambda-, trp-, lac-, and Ipp-derived Promoters”, in Rodriguez and Denhardt (eds.) Vectors: A Survey of Molecular Cloning Vectors and Their Uses 10:205-236 Buttersworth, Boston, Mass.

Lower eukaryotes, e.g., yeasts and Dictyostelium, may be transformed with an HCC5 chemokine; a Dub11 or Dub12 gene; or an MD1 or MD2 sequence containing vectors. For purposes of this invention, the most common lower eukaryotic host is the baker's yeast, Saccharomyces cerevisiae. It will be used generically to represent lower eukaryotes although a number of other strains and species are also available. Yeast vectors typically consist of a replication origin (unless of the integrating type), a selection gene, a promoter, DNA encoding the desired protein or its fragments, and sequences for translation termination, polyadenylation, and transcription termination. Suitable expression vectors for yeast include such constitutive promoters as 3-phosphoglycerate kinase and various other glycolytic enzyme gene promoters or such inducible promoters as the alcohol dehydrogenase 2 promoter or metallothionine promoter. Suitable vectors include derivatives of the following types: self-replicating low copy number (such as the YRp-series), self-replicating high copy number (such as the YEp-series); integrating types (such as the YIp-series), or mini-chromosomes (such as the YCp-series).

Higher eukaryotic tissue culture cells are typically the preferred host cells for expression of the functionally active HCC5 chemokine protein. In principle, many higher eukaryotic tissue culture cell lines may be used, e.g., insect baculovirus expression systems, whether from an invertebrate or vertebrate source. However, mammalian cells are preferred to achieve proper processing, both cotranslationally and posttranslationally. Transformation or transfection and propagation of such cells is routine. Useful cell lines include HeLa cells, Chinese hamster ovary (CHO) cell lines, baby rat kidney (BRK) cell lines, insect cell lines, bird cell lines, and monkey (COS) cell lines. Expression vectors for such cell lines usually include an origin of replication, a promoter, a translation initiation site, RNA splice sites (e.g., if genomic DNA is used), a polyadenylation site, and a transcription termination site. These vectors also may contain a selection gene or amplification gene. Suitable expression vectors may be plasmids, viruses, or retroviruses carrying promoters derived, e.g., from such sources as from adenovirus, SV40, parvoviruses, vaccinia virus, or cytomegalovirus. Representative examples of suitable expression vectors include pcDNA1; pCD, see Okayama, et al. (1985) Mol. Cell Biol. 5:1136-1142; pMC1neo Poly-A, see Thomas, et al. (1987) Cell 51:503-512; and baculovirus vectors such as pAC 373 or pAC 610. See, e.g., Miller (1988) Ann. Rev. Microbiol. 42:177-199. Usually, expression vectors are designed for stable replication in their host cells or for amplification to greatly increase the total number of copies of the desirable gene per cell. It is not always necessary to require that an expression vector replicate in a host cell, e.g., it is possible to effect transient expression of the antigen or its fragments in various hosts using vectors that do not contain a replication origin that is recognized by the host cell. It is also possible to use vectors that cause integration of a gene or its fragments into the host DNA by recombination, or to integrate a promoter which controls expression of an endogenous gene. See, e.g., Treco, et al. WO96/29411 or U.S. Ser. No. 08/406,030.

Adenovirus techniques are available for expression of the genes in various cells and organs. See, e.g., Hitt, et al. (1997) Adv. Pharmacol. 40:137-195; and literature from Quantum Biotechnologies, Montreal, Canada. Animals may be useful to determine the effects of the gene on various developmental or physiologically functional animal systems.

It is likely that an HCC5 chemokine; a Dub11 or Dub12 protein; or an MD1 or MD2 protein need not be glycosylated to elicit biological responses. However, it will occasionally be desirable to express an HCC5 chemokine; a Dub11 or Dub12 polypeptide; or an MD1 or MD2 polypeptide in a system which provides a specific or defined glycosylation pattern. In this case, the usual pattern will be that provided naturally by the expression system. However, the pattern will be modifiable by exposing the polypeptide, e.g., in unglycosylated form, to appropriate glycosylating proteins introduced into a heterologous expression system. See, e.g., Luckow and Summers (1988) Bio/Technology 6:47-55; and Kaufman (1990) Meth. Enzymol. 185:487-511. Preferred prokaryotic forms lack eukaryotic glycosylation patterns. However, the pattern will be modifiable by exposing the polypeptide, e.g., an unglycosylated form, to appropriate glycosylating proteins introduced into a heterologous expression system. For example, the desired gene may be cotransformed with one or more genes encoding mammalian or other glycosylating enzymes. Using this approach, certain mammalian glycosylation patterns will be achievable or approximated in prokaryote or other cells. It is further understood that over glycosylation may be detrimental to HCC5 chemokine; Dub11 or Dub12 protein; or an MD1 or MD2 protein biological activity, and that one of skill may perform routine testing to optimize the degree of glycosylation which confers optimal biological activity.

An HCC5 chemokine; a Dub11 or Dub12 protein; or an MD1 or MD2 protein, or a fragment thereof, may be engineered to be phosphatidyl inositol (PI) linked to a cell membrane, but can be removed from membranes by treatment with a phosphatidyl inositol cleaving enzyme, e.g., phosphatidyl inositol phospholipase-C. This releases the antigen in a biologically active form, and allows purification by standard procedures of protein chemistry. See, e.g., Low (1989) Biochem. Biophys. Acta 988:427-454; Tse, et al. (1985) Science 230:1003-1008; and Brunner, et al. (1991) J. Cell Biol. 114:1275-1283.

Transformed cells include cells, preferably mammalian, that have been transformed or transfected with vectors containing a prostaglandin transporter gene, typically constructed using recombinant DNA techniques. Transformed host cells usually express the antigen or its fragments, but for purposes of cloning, amplifying, and manipulating its DNA, do not need to express the protein. This invention further contemplates culturing transformed cells in a nutrient medium, thus permitting the protein, or soluble fragments, to accumulate in the culture. Soluble protein can be recovered, either from the culture or from the culture medium, and membrane associated proteins may be prepared from suitable cell subfractions.

Now that HCC5 chemokines; Dub11 or Dub12 proteins; or MD1 or MD2 proteins have been characterized, fragments or derivatives thereof can be prepared by conventional processes for synthesizing peptides. These include processes such as are described in Stewart and Young (1984) Solid Phase Peptide Synthesis Pierce Chemical Co., Rockford, Ill.; Bodanszky and Bodanszky (1984) The Practice of Peptide Synthesis Springer-Verlag, New York, N.Y.; and Bodanszky (1984) The Principles of Peptide Synthesis Springer-Verlag, New York, N.Y. For example, an azide process, an acid chloride process, an acid anhydride process, a mixed anhydride process, an active ester process (for example, p-nitrophenyl ester, N-hydroxysuccinimide ester, or cyanomethyl ester), a carbodiimidazole process, an oxidative-reductive process, or a dicyclohexylcarbodiimide (DCCD)/additive process can be used. Solid phase and solution phase syntheses are both applicable to the foregoing processes. See also chemical ligation, e.g., Dawson, et al. (1994) Science 266:776-779, a method of linking long synthetic peptides by a peptide bond.

An amino group-protected amino acid is bound in sequence through condensation of its activated carboxyl group and the reactive amino group of the previously formed peptide or chain, to synthesize the peptide step by step. After synthesizing the complete sequence, the peptide is split off from the insoluble carrier to produce the peptide. This solid-phase approach is generally described by Merrifield et al. (1963) in J. Am. Chem. Soc. 85:2149-2156, which is incorporated herein by reference.

The prepared protein and fragments thereof can be isolated and purified from the reaction mixture by means of peptide separation, e.g., by extraction, precipitation, electrophoresis and various forms of chromatography, and the like. The HCC5 chemokine; Dub11 or Dub12 proteins; or an MD1 or MD2 proteins of this invention can be obtained in varying degrees of purity depending upon its desired use. Purification can be accomplished by use of known protein purification techniques or by the use of the antibodies or binding partners herein described, e.g., in immunoabsorbent affinity chromatography. This immunoabsorbent affinity chromatography is carried out by first linking the antibodies to a solid support and then contacting the linked antibodies with solubilized lysates of appropriate source cells, lysates of other cells expressing the ligand, or lysates or supernatants of cells producing the HCC5 chemokine; Dub11 or Dub12 protein; or MD1 or MD2 protein as a result of recombinant DNA techniques, see below.

Multiple cell lines may be screened for one which expresses, or alternatively does not express, an HCC5 chemokine; a Dub11 or Dub12 protein; or an MD1 or MD2 protein at a different level compared with other cells. Natural HCC5 chemokines; Dub11 or Dub12 proteins; or MD1 or MD2 proteins can be isolated from natural sources, or by expression from a transformed cell using an appropriate expression vector. Purification of the expressed protein is achieved by standard procedures, or may be combined with engineered means for effective purification at high efficiency from cell lysates or supernatants. Epitope or other tags, e.g., FLAG or His₆ segments, can be used for such purification features.

V. Antibodies

Antibodies can be raised to various HCC5 chemokines; Dub11 or Dub12 polypeptides; or MD1 or MD2 polypeptides, including individual, polymorphic, allelic, strain, or species variants, and fragments thereof, both in their naturally occurring (full-length) forms and in their recombinant forms. Additionally, antibodies can be raised to the polypeptides in their active forms or in their inactive forms, or in native or denatured forms. Anti-idiotypic antibodies may also be used. The antibodies may exhibit various binding specificities for species, individual or polymorphic variants

A. Antibody Production

A number of immunogens may be used to produce antibodies reactive with an HCC5 chemokine; a Dub11 or Dub12 protein; or an MD1 or MD2 protein. Recombinant or purified protein is the preferred immunogen for the production of monoclonal or polyclonal antibodies. Naturally occurring protein may also be used either in pure or impure form. Synthetic peptides, made using, e.g., the HCC5 chemokine; Dub11 or Dub12 polypeptide; or MD1 or MD2 polypeptide sequences described herein, may also be used as an immunogen for the production of antibodies to each respective antigen. Recombinant protein can be expressed in eukaryotic or prokaryotic cells as described herein, and purified as described. Naturally folded or denatured material can be used, as appropriate, for producing antibodies. Either monoclonal or polyclonal antibodies may be generated for subsequent use in immunoassays to qualitatively or quantitatively measure the protein.

Methods of producing polyclonal antibodies are known to those of skill in the art. Typically, an immunogen, preferably a purified protein, is mixed with an adjuvant and animals are immunized with the mixture. The animal's immune response to the immunogen preparation is monitored by taking test bleeds and determining the titer of reactivity to antigen of interest. When appropriately high titers of antibody to the immunogen are obtained, usually after repeated immunizations, blood is collected from the animal and antisera are prepared. Further fractionation of the antisera to enrich for IgG or antibodies reactive to the protein can be performed if desired. See, e.g., Harlow and Lane; or Coligan.

Monoclonal antibodies may be obtained by various techniques familiar to those skilled in the art. Typically, spleen cells from an animal immunized with a desired antigen are immortalized, commonly by fusion with a myeloma cell. See, Kohler and Milstein (1976) Eur. J. Immunol. 6:511-519, incorporated herein by reference. Alternative methods of immortalization include transformation with Epstein Barr Virus, oncogenes, or retroviruses, or other methods known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Antibodies, including binding fragments and single chain versions, against predetermined fragments of the desired antigen can be raised by immunization of animals with conjugates of the fragments with carrier proteins as described above. Monoclonal antibodies are prepared from cells secreting the desired antibody. These antibodies can be screened for binding to normal or defective HCC5 chemokine; Dub11 or Dub12 proteins; or MD1 or MD2 proteins, or screened for agonistic or antagonistic activity, e.g., mediated through a receptor. These monoclonal antibodies will usually bind with at least a KD of about 1 mM, more usually at least about 300 μM, typically at least about 10 μM, more typically at least about 30 μM, preferably at least about 10 μM, and more preferably at least about 3 μM or better.

In some instances, it is desirable to prepare monoclonal antibodies from various mammalian hosts, such as mice, rodents, primates, humans, etc. Description of techniques for preparing such monoclonal antibodies may be found in, e.g., Stites, et al. (eds.) Basic and Clinical Immunology (4th ed.) Lange Medical Publications, Los Altos, Calif., and references cited therein; Harlow and Lane (1988) Antibodies: A Laboratory Manual CSH Press; Goding (1986) Monoclonal Antibodies: Principles and Practice (2d ed.) Academic Press, New York, N.Y.; and particularly in Kohler and Milstein (1975) Nature 256:495-497, which discusses one method of generating monoclonal antibodies. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve selection of libraries of antibodies in phage or similar vectors. See, e.g., Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors; fluorescent moieties, chemiluminescent moieties, magnetic particles, and the like. Patents, teaching the use of such labels include U.S. Pat. Nos. 3,817,837; 3,850,752; 3,939,350; 3,996,345; 4,277,437; 4,275,149; and 4,366,241. Also, recombinant or chimeric immunoglobulins may be produced, see Cabilly, U.S. Pat. No. 4,816,567; or made in transgenic mice, see Mendez, et al. (1997) Nature Genetics 15:146-156. These references are incorporated herein by reference.

The antibodies of this invention are useful for affinity chromatography in isolating an HCC5 chemokine; a Dub11 or Dub12 protein; or an MD1 or MD2 protein. Columns can be prepared where the antibodies are linked to a solid support, e.g., particles, such as agarose, SEPHADEX, or the like, where a cell lysate or supernatant may be passed through the column, the column washed, followed by increasing concentrations of a mild denaturant, whereby purified antigen will be released. Likewise, antibody binding to the chemokine may be capable of neutralizing receptor binding, and may serve as a receptor antagonist. They may also be useful as Western blot detection reagents, or ELISA reagents.

The antibodies may also be used to screen expression libraries for particular expression products. Usually the antibodies used in such a procedure will be labeled with a moiety allowing easy detection of presence of antigen by antibody binding.

Antibodies to a selected antigen may be used for the identification of cell populations expressing such protein. By assaying the expression products of cells expressing an HCC5 chemokine; a Dub11 or Dub12 protein; or an MD1 or MD2 protein it is possible to diagnose disease, e.g., immune-compromised conditions.

Antibodies raised against the various antigens will also be useful to raise anti-idiotypic antibodies. These will be useful in detecting or diagnosing various immunological conditions related to expression of the antigens.

B. Immunoassays

A particular protein can be measured by a variety of immunoassay methods. For a review of immunological and immunoassay procedures in general, see Stites and Terr (eds. 1991) Basic and Clinical Immunology (7th ed.). Moreover, the immunoassays of the present invention can be performed in many configurations, which are reviewed extensively in Maggio (ed. 1980) Enzyme Immunoassay CRC Press, Boca Raton, Fla.; Tijan (1985) “Practice and Theory of Enzyme Immunoassays,” Laboratory Techniques in Biochemistry and Molecular Biology, Elsevier Science Publishers B.V., Amsterdam; and Harlow and Lane Antibodies, A Laboratory Manual, supra, each of which is incorporated herein by reference. See also Chan (ed. 1987) Immunoassay: A Practical Guide Academic Press, Orlando, Fla.; Price and Newman (eds. 1991) Principles and Practice of Immunoassays Stockton Press, NY; and Ngo (ed. 1988) Non-isotopic Immunoassays Plenum Press, NY.

Immunoassays for measurement of an HCC5 chemokine; a Dub11 or Dub12 protein; or an MD1 or MD2 protein can be performed by a variety of methods known to those skilled in the art. In brief, immunoassays to measure the protein can be either competitive or noncompetitive binding assays. In competitive binding assays, the sample to be analyzed competes with a labeled analyte for specific binding sites on a capture agent bound to a solid surface. Preferably the capture agent is an antibody specifically reactive with, e.g., an HCC5 chemokine; a Dub11 or Dub12 protein; or an MD1 or MD2 protein produced as described above. The concentration of labeled analyte bound to the capture agent is inversely proportional to the amount of free analyte present in the sample.

In a competitive binding immunoassay, e.g., an HCC5 chemokine; a Dub11 or Dub12 protein; or an MD1 or MD2 protein present in the sample competes with labeled protein for binding to a specific binding agent, for example, an antibody specifically reactive with an HCC5 chemokine; a Dub11 or Dub12 protein; or an MD1 or MD2 protein. The binding agent may be bound to a solid surface to effect separation of bound labeled protein from the unbound labeled protein. Alternately, the competitive binding assay may be conducted in liquid phase and a variety of techniques known in the art may be used to separate the bound labeled protein from the unbound labeled protein. Following separation, the amount of bound labeled protein is determined. The amount of protein present in the sample is inversely proportional to the amount of labeled protein binding.

Alternatively, a homogeneous immunoassay may be performed in which a separation step is not needed. In these immunoassays, the label on the protein is altered by the binding of the protein to its specific binding agent. This alteration in the labeled protein results in a decrease or increase in the signal emitted by label, so that measurement of the label at the end of the immunoassay allows for detection or quantitation of the protein.

An HCC5 chemokine; a Dub11 or Dub12 protein; or an MD1 or MD2 protein may also be determined by a variety of noncompetitive immunoassay methods. For example, a two-site, solid phase sandwich immunoassay may be used. In this type of assay, a binding agent for the protein, for example an antibody, is attached to a solid support. A second protein binding agent, which may also be an antibody, and which binds the protein at a different site, is labeled. After binding at both sites on the protein has occurred, the unbound labeled binding agent is removed and the amount of labeled binding agent bound to the solid phase is measured. The amount of labeled binding agent bound is directly proportional to the amount of protein in the sample.

Western blot analysis can be used to determine the presence of, e.g., an HCC5 chemokine; a Dub11 or Dub12 protein; or an MD1 or MD2 protein in a sample. Electrophoresis is carried out, for example, on a tissue sample suspected of containing the protein. Following electrophoresis to separate the proteins, and transfer of the proteins to a suitable solid support, e.g., a nitrocellulose filter, the solid support is incubated with an antibody reactive with the protein. This antibody may be labeled, or alternatively may be detected by subsequent incubation with a second labeled antibody that binds the primary antibody.

The immunoassay formats described above employ labeled assay components. The label may be coupled directly or indirectly to the desired component of the assay according to methods well known in the art. A wide variety of labels and methods may be used. Traditionally, a radioactive label incorporating ³H, ¹²⁵I, ³⁵S, ¹⁴C, or ³²P was used. Non-radioactive labels include ligands which bind to labeled antibodies, fluorophores, chemiluminescent agents, enzymes, and antibodies which can serve as specific binding pair members for a labeled ligand. The choice of label depends on sensitivity required, ease of conjugation with the compound, stability requirements, and available instrumentation. For a review of various labeling or signal producing systems which may be used, see U.S. Pat. No. 4,391,904, which is incorporated herein by reference.

Antibodies reactive with a particular protein can also be measured by a variety of immunoassay methods. For a review of immunological and immunoassay procedures applicable to the measurement of antibodies by immunoassay techniques, see Stites and Terr (eds.) Basic and Clinical Immunology (7th ed.) supra; Maggio (ed.) Enzyme Immunoassay, supra; and Harlow and Lane Antibodies: A Laboratory Manual, supra.

In brief, immunoassays to measure antisera reactive with, e.g., an HCC5 chemokine; a Dub11 or Dub12 protein; or an MD1 or MD2 protein can be either competitive or noncompetitive binding assays. In competitive binding assays, the sample analyte competes with a labeled analyte for specific binding sites on a capture agent bound to a solid surface. Preferably the capture agent is a purified recombinant HCC5 chemokine; Dub11 or Dub12 protein; or MD1 or MD2 protein produced as described above. Other sources of HCC5 chemokine; Dub11 or Dub12 protein; or MD1 or MD2 protein, including isolated or partially purified naturally occurring protein, may also be used. Noncompetitive assays include sandwich assays, in which the sample analyte is bound between two analyte-specific binding reagents. One of the binding agents is used as a capture agent and is bound to a solid surface. The second binding agent is labeled and is used to measure or detect the resultant complex by visual or instrument means. A number of combinations of capture agent and labeled binding agent can be used. A variety of different immunoassay formats, separation techniques, and labels can be also be used similar to those described above for the measurement of an HCC5 chemokine; a Dub11 or Dub12 protein; or an MD1 or MD2 protein.

VI. Purified Protein

Primate, e.g., human, HCC5 nucleotide and amino acid sequences are provided in SEQ ID NO: 1, and 2. Primate, e.g., human, Dub11 and Dub12 nucleotide and amino acid sequences are provided respectively in SEQ ID NO: 8 or 10, and 12 or 14; 9 or 11, and 13 or 15. Primate, e.g., human, MD1 and MD2 nucleotide and amino acid sequences are provided respectively in SEQ ID NO: 18 and 20 or 22; 19 and 21 or 23. Rodent, e.g., mouse, MD2 nucleotide and amino acid sequences are provided in SEQ ID NO: 24 and 25 or 26.

Purified protein or defined peptides are useful for generating antibodies by standard methods, as described above. Synthetic peptides or purified protein can be presented to an immune system to generate polyclonal and monoclonal antibodies. See, e.g., Coligan (1991) Current Protocols in Immunology Wiley/Greene, NY; and Harlow and Lane (1989) Antibodies: A Laboratory Manual Cold Spring Harbor Press, NY, which are incorporated herein by reference. Alternatively, a HCC5 chemokine or MD1 or MD2 receptor can be useful as a specific binding reagent, and advantage can be taken of its specificity of binding, for, e.g., purification of a HCC5 chemokine ligand or a or MD1 or MD2 ligand.

The specific binding composition can be used for screening an expression library made from a cell line which expresses an HCC5 chemokine; or an MD1 or MD2 protein . Many methods for screening are available, e.g., standard staining of surface expressed ligand, or by panning. Screening of intracellular expression can also be performed by various staining or immunofluorescence procedures. The binding compositions could be used to affinity purify or sort out cells expressing the ligand.

The peptide segments, along with comparison to homologous genes, can also be used to produce appropriate oligonucleotides to screen a library. The genetic code can be used to select appropriate oligonucleotides useful as probes for screening. In combination with polymerase chain reaction (PCR) techniques, synthetic oligonucleotides will be useful in selecting desired clones from a library, including natural allelic and polymorphic variants. Complementary sequences will also be used as probes, primers, or antisense strands. Various fragments should be particularly useful, e.g., coupled with anchored vector or poly-A complementary PCR techniques or with complementary DNA of other peptides.

The peptide sequences allow preparation of peptides to generate antibodies to recognize such segments, and allow preparation of oligonucleotides which encode such sequences. The sequence also allows for synthetic preparation, e.g., see Dawson, et al. (1994) Science 266:776-779. Since HCC5 and MD1 and MD2 will typically be secreted proteins, the genes will normally possess an N-terminal signal sequence, which is removed upon processing and secretion. However, the exact processing point may be vary in different cell types, and forms of different lengths are often detected. Prediction of the signal cleavage point can be performed, e.g., using the methods of Nielsen, et al. (1997) Protein Eng. 10:1-8. Analysis of the structural features in comparison with the most closely related reported sequences has revealed similarities with other cytokines, particularly the class of proteins known as CC and CXC chemokines.

VII. Physical Variants

This invention also encompasses proteins or peptides having substantial amino acid sequence similarity with an amino acid sequence of an HCC5 chemokine; a Dub11 or Dub12 protein; or an MD1 or MD2 protein. Natural variants include individual, polymorphic, allelic, strain, or species variants.

Amino acid sequence similarity, or sequence identity, is determined by optimizing residue matches, if necessary, by introducing gaps as required. This changes when considering conservative substitutions as matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. Homologous amino acid sequences include natural polymorphic, allelic, and interspecies variations in the protein sequence. Typical homologous proteins or peptides will have from 50-100% similarity (if gaps can be introduced), to 75-100% similarity (if conservative substitutions are included) with the amino acid sequence of, e.g., the HCC5 chemokine. Similarity measures will be at least about 50%, generally at least 60%, more generally at least 65%, usually at least 70%, more usually at least 75%, preferably at least 80%, and more preferably at least 80%, and in particularly preferred embodiments, at least 85% or more. See also Needleham, et al. (1970) J. Mol. Biol. 48:443-453; Sankoff, et al. (1983) Time Warps, String Edits, and Macromolecules: The Theory and Practice of Sequence Comparison Chapter One, Addison-Wesley, Reading, Mass.; and software packages from IntelliGenetics, Mountain View, Calif.; and the University of Wisconsin Genetics Computer Group, Madison, Wis. See also discussion of identity measures, above.

Nucleic acids encoding, e.g., an HCC5 chemokine; a Dub11 or Dub12 polypeptide; or an MD1 or MD2 polypeptide will typically hybridize respectively to the nucleic acid sequence of SEQ ID NO: 1; 8 or 10; 12 or 14; 20 or 22; or 24 under stringent conditions. For example, nucleic acids encoding HCC5 chemokine proteins will normally hybridize to the nucleic acid of SEQ ID NO: 1 under stringent hybridization conditions. Generally, stringent conditions are selected to be about 10° C. lower than the thermal melting point (Tm) for the probe sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.2 molar at pH 7 and the temperature is at least about 50° C. Other factors may significantly affect the stringency of hybridization, including, among others, base composition and size of the complementary strands, the presence of organic solvents such as formamide, and the extent of base mismatching. A preferred embodiment will include nucleic acids which will bind to disclosed sequences in 50% formamide and 200 mM NaCl at 55° C.

An isolated HCC5 chemokine; Dub11 or Dub12; or an MD1 or MD2 DNA can be readily modified by nucleotide substitutions, nucleotide deletions, nucleotide insertions, and short inversions of nucleotide stretches. These modifications result in novel DNA sequences which encode an HCC5 chemokine; a Dub11 or Dub12; or an MD1 or MD2 antigens, their derivatives, or proteins having highly similar physiological, immunogenic, or antigenic activity.

Modified sequences can be used to produce mutant antigens or to enhance expression. Enhanced expression may involve gene amplification, increased transcription, increased translation, and other mechanisms. Such mutant HCC5 chemokine; Dub11 or Dub12 polypeptide; or MD1 or MD2 derivatives include predetermined or site-specific mutations of the protein or its fragments. “Mutant HCC5 chemokine; Dub11 or Dub12 protein; or MD1 or MD2 protein” encompasses a polypeptide otherwise falling within the homology definition of the human HCC5 chemokine; Dub11 or Dub12 protein; or MD1 or MD2 protein as set forth above, but having an amino acid sequence which differs from that of an HCC5 chemokine; a Dub11 or Dub12 protein; or an MD1 or MD2 protein as found in nature, whether by way of deletion, substitution, or insertion. In particular, “site specific mutant HCC5 chemokine; Dub11 or Dub12 protein; or MD1 or MD2 protein” generally includes proteins having significant similarity with a protein having a sequence of SEQ ID NO: 2; 9 or 11; 13 or 15; 21 or 23; 25 or 26, e.g., natural embodiments, and as sharing various biological activities, e.g., antigenic or immunogenic, with those sequences, and in preferred embodiments contain most or all of the disclosed sequence. This applies also to polymorphic variants from different individuals. Similar concepts apply to different HCC5 chemokine; Dub11 or Dub12 protein; or MD1 or MD2 proteins, particularly those found in various warm blooded animals, e.g., mammals and birds. As stated before, it is emphasized that descriptions are generally meant to encompass other HCC5 chemokine; Dub11 or Dub12 protein; or MD1 or MD2 proteins, not limited to the specific HCC5 chemokine; Dub11 or Dub12 protein; or MD1 or MD2 proteins or the human or rodent embodiments specifically discussed.

Although site specific mutation sites are predetermined, mutants need not be site specific. HCC5 chemokine; Dub11 or Dub12 protein; or MD1 or MD2 protein mutagenesis can be conducted by making amino acid insertions or deletions. Substitutions, deletions, insertions, or any combinations may be generated to arrive at a final construct. Insertions include amino- or carboxyl-terminal fusions, e.g., epitope tags. Random mutagenesis can be conducted at a target codon and the expressed mutants can then be screened for the desired activity. Methods for making substitution mutations at predetermined sites in DNA having a known sequence are well known in the art, e.g., by M13 primer mutagenesis or polymerase chain reaction (PCR) techniques. See also, Sambrook, et al. (1989) and Ausubel, et al. (1987 and Supplements). The mutations in the DNA normally should not place coding sequences out of reading frames and preferably will not create complementary regions that could hybridize to produce secondary mRNA structure such as loops or hairpins.

The present invention also provides recombinant proteins, e.g., heterologous fusion proteins using segments from these proteins. A heterologous fusion protein is a fusion of proteins or segments which are naturally not normally fused in the same manner. Thus, the fusion product of an immunoglobulin with, e.g., an HCC5 chemokine; a Dub11 or Dub12 polypeptide; or an MD1 or MD2 polypeptide is a continuous protein molecule having sequences fused in a typical peptide linkage, typically made as a single translation product and exhibiting properties derived from each source peptide. A similar concept applies to heterologous nucleic acid sequences.

In addition, new constructs may be made from combining similar functional domains from other proteins. For example, protein-binding or other segments may be “swapped” between different new fusion polypeptides or fragments. See, e.g., Cunningham, et al. (1989) Science 243:1330-1336; and O'Dowd, et al. (1988) J. Biol. Chem. 263:15985-15992. Thus, new chimeric polypeptides exhibiting new combinations of specificities will result from the functional linkage of protein-binding specificities and other functional domains.

VIII. Binding Agent:Protein Complexes

An HCC5 chemokine; a Dub11 or Dub12 protein; or an MD1 or MD2 protein that specifically, or selectively, binds to or that is specifically immunoreactive with an antibody generated against a defined immunogen, such as an immunogen consisting of the amino acid sequence of SEQ ID NO: 2; 9 or 11; 13 or 15; 21 or 23; 25 or 26 is typically determined in an immunoassay. The immunoassay uses a polyclonal antiserum which was raised, e.g., to a protein of SEQ ID NO: 2; 9 or 11; 13 or 15; 21 or 23; 25 or 26. This antiserum is selected to have low crossreactivity against other chemokines and any such crossreactivity may be removed by immunoabsorption prior to use in the immunoassay.

To produce antisera for use in an immunoassay, the protein of SEQ ID NO: 2; 9 or 11; 13 or 15; 21 or 23; 25 or 26, is isolated as described herein. For example, recombinant protein may be produced in a mammalian cell line. An inbred strain of mice such as Balb/c is immunized with the protein of SEQ ID NO: 2; 9 or 11; 13 or 15; 21 or 23; 25 or 26, using a standard adjuvant, such as Freund's adjuvant, and a standard mouse immunization protocol (see Harlow and Lane, supra). Alternatively, a synthetic peptide, preferably near full length, derived from the sequences disclosed herein and conjugated to a carrier protein can be used an immunogen. Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross reactivity, e.g., against C, CC, CX3C, and CXC chemokines, using a competitive binding immunoassay such as the one described in Harlow and Lane, supra, at pages 570-573. Preferably two chemokines are used in this determination in conjunction with primate HCC5 chemokine.

Immunoassays in the competitive binding format can be used for the crossreactivity determinations. For example, a protein of SEQ ID NO: 2; 9 or 11; 13 or 15; 21 or 23; 25 or 26 can be immobilized to a solid support. Proteins added to the assay compete with the binding of the antisera to the immobilized antigen. The ability of the above proteins to compete with the binding of the antisera to the immobilized protein is compared to the protein of SEQ ID NO: 2; 9 or 11; 13 or 15; 21 or 23; 25 or 26. The percent crossreactivity for the above proteins is calculated, using standard calculations. Those antisera with less than 10% crossreactivity with each of the proteins listed above are selected and pooled. The cross-reacting antibodies are then removed from the pooled antisera by immunoabsorption with the above-listed proteins, e.g., HCC1, HCC2, HCC3, and HCC4.

The immunoabsorbed and pooled antisera are then used in a competitive binding immunoassay as described above to compare a second protein to the immunogen protein (e.g., the HCC5 chemokine motif of SEQ ID NO: 2). To make this comparison, the two proteins are each assayed at a wide range of concentrations and the amount of each protein required to inhibit 50% of the binding of the antisera to the immobilized protein is determined. If the amount of the second protein required is less than twice the amount of the protein, e.g., of SEQ ID NO: 2 that is required, then the second protein is the to specifically bind to an antibody generated to the immunogen.

It is understood that an HCC5 chemokine; a Dub11 or Dub12 protein; or an MD1 or MD2 protein is a species form of a group of homologous proteins across species that include closely related genes. For a particular gene product, such as the HCC5 chemokine; the Dub11 or Dub12 protein; or the MD1 or MD2 protein, the term refers not only to the amino acid sequences disclosed herein, but also to other proteins that are polymorphic, allelic, or non-allelic variants. It is also understood that the terms “an HCC5 chemokine”; “a Dub11 or Dub12 protein”; or “an MD1 or MD2 protein” includes nonnatural mutations introduced by deliberate mutation using conventional recombinant technology such as single site mutation, or by excising short sections of DNA encoding an HCC5 chemokine; a Dub11 or Dub12 protein; or an MD1 or MD2 protein, or by substituting new amino acids, or adding new amino acids. Such minor alterations should substantially maintain the immunoidentity of the original molecule and/or its biological activity. Thus, these alterations include proteins that are specifically immunoreactive with a designated naturally occurring HCC5 chemokine; Dub11 or Dub12 protein; or MD1 or MD2 protein, e.g., the HCC5 chemokine protein shown in SEQ ID NO: 2. The biological properties of the altered proteins can be determined by expressing the protein in an appropriate cell line and measuring an appropriate biological activity, e.g., a chemotactic effect. Particular protein modifications considered minor would include conservative substitution of amino acids with similar chemical properties, as described above for the HCC5 chemokine as a whole. By aligning a protein optimally with the appropriate protein and by using the conventional immunoassays described herein to determine immunoidentity, or by using lymphocyte chemotaxis assays, one can determine the protein compositions of the invention.

IX. Functional Variants

The blocking of physiological response to an HCC5 chemokine; a Dub11 or Dub12 protein; or an MD1 or MD2 protein may result from the inhibition of binding of the protein to its binding partner, e.g., through competitive inhibition. Thus, in vitro assays of the present invention will often use isolated protein, membranes from cells expressing a recombinant membrane associated HCC5 chemokine; Dub11 or Dub12 protein; or MD1 or MD2 protein, soluble fragments comprising receptor binding segments of these proteins, or fragments attached to solid phase substrates. These assays will also allow for the diagnostic determination of the effects of either binding segment mutations and modifications, or protein mutations and modifications, e.g., protein analogs. This invention also contemplates the use of competitive drug screening assays, e.g., where neutralizing antibodies to antigen or receptor fragments compete with a test compound for binding to the protein. In this manner, the antibodies can be used to detect the presence of a polypeptide which shares one or more antigenic binding sites of the protein and can also be used to occupy binding sites on the protein that might otherwise interact with a receptor.

“Derivatives” of HCC5 chemokine; Dub11 or Dub12 protein; or MD1 or MD2 antigens include amino acid sequence mutants, glycosylation variants, and covalent or aggregate conjugates with other chemical moieties. Covalent derivatives can be prepared by linkage of functionalities to groups which are found in an HCC5 chemokine; a Dub11 or Dub12 protein; or an MD1 or MD2 protein amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including, e.g., C3 to C18 normal alkyl, thereby forming alkanoyl aroyl species. Covalent attachment to carrier proteins may be important when immunogenic moieties are haptens.

In particular, glycosylation alterations are included, e.g., made by modifying the glycosylation patterns of a polypeptide during its synthesis and processing, or in further processing steps. Particularly preferred means for accomplishing this are by exposing the polypeptide to glycosylating enzymes derived from cells which normally provide such processing, e.g., mammalian glycosylation enzymes. Deglycosylation enzymes are also contemplated. Also embraced are versions of the same primary amino acid sequence which have other minor modifications, including phosphorylated amino acid residues, e.g., phosphotyrosine, phosphoserine, or phosphothreonine, or other moieties, including ribosyl groups or cross-linking reagents.

A major group of derivatives are covalent conjugates of an HCC5 chemokine; a Dub11 or Dub12 protein; or an MD1 or MD2 protein or fragments thereof with other proteins or polypeptides. These derivatives can be synthesized in recombinant culture such as N- or C-terminal fusions or by the use of agents known in the art for their usefulness in cross-linking proteins through reactive side groups. Preferred protein derivatization sites with cross-linking agents are at free amino groups, carbohydrate moieties, and cysteine residues.

Fusion polypeptides between an HCC5 chemokine; a Dub11 or Dub12 protein; or an MD1 or MD2 protein and other homologous or heterologous proteins are also provided. Many growth factors and cytokines are homodimeric entities, and a repeat construct may have various advantages, including lessened susceptibility to proteolytic degradation. Moreover, many receptors require ligand dimerization to transduce a signal, and various dimeric proteins or domain repeats can be desirable. Heterologous polypeptides may be fusions between different surface markers, resulting in, e.g., a hybrid protein exhibiting receptor binding specificity. Likewise, heterologous fusions may be constructed which would exhibit a combination of properties or activities of the derivative proteins. Typical examples are fusions of a reporter polypeptide, e.g., luciferase, with a segment or domain of a protein, e.g., a receptor-binding segment, so that the presence or location of the fused protein may be easily determined. See, e.g., Dull, et al., U.S. Pat. No. 4,859,609. Other gene fusion partners include bacterial β-galactosidase, trpE, Protein A, β-lactamase, alpha amylase, alcohol dehydrogenase, and yeast alpha mating factor. See, e.g., Godowski, et al. (1988) Science 241:812-816.

Such polypeptides may also have amino acid residues which have been chemically modified by phosphorylation, sulfonation, biotinylation, or the addition or removal of other moieties, particularly those which have molecular shapes similar to phosphate groups. In some embodiments, the modifications will be useful labeling reagents, or serve as purification targets, e.g., affinity ligands.

This invention also contemplates the use of derivatives of HCC5 chemokine; Dub11 or Dub12 protein; or MD1 or MD2 proteins other than variations in amino acid sequence or glycosylation. Such derivatives may involve covalent or aggregative association with chemical moieties. These derivatives generally fall into the three classes: (1) salts, (2) side chain and terminal residue covalent modifications, and (3) adsorption complexes, for example with cell membranes. Such covalent or aggregative derivatives are useful as immunogens, as reagents in immunoassays, or in purification methods such as for affinity purification of ligands or other binding ligands. For example, an HCC5 chemokine; a Dub11 or Dub12 protein; or an MD1 or MD2 antigen can be immobilized by covalent bonding to a solid support such as cyanogen bromide-activated SEPHAROSE, by methods which are well known in the art, or adsorbed onto polyolefin surfaces, with or without glutaraldehyde cross-linking, for use in the assay or purification of anti-HCC5 chemokine; anti-Dub11 or Dub12 protein; or anti-MD1 or MD2 protein antibodies or their binding partners. The HCC5 chemokine; Dub11 or Dub12 protein; or MD1 or MD2 proteins can also be labeled with a detectable group, e.g., radioiodinated by the chloramine T procedure, covalently bound to rare earth chelates, or conjugated to another fluorescent moiety fQr use in diagnostic assays. Purification of an HCC5 chemokine; a Dub11 or Dub12 protein; or an MD1 or MD2 protein may be effected by immobilized antibodies or receptor.

Isolated HCC5 chemokine; Dub11 or Dub12 protein; or MD1 or MD2 genes will allow transformation of cells, e.g., either species types or cells which lack corresponding proteins and exhibit negative background activity. Expression of transformed genes will allow isolation of antigenically pure cell lines, with defined or single specie variants. This approach will allow, for example, more sensitive detection and discrimination of the physiological effects of HCC5 chemokine receptor proteins. Subcellular fragments, e.g., cytoplasts or membrane fragments, can be isolated and used.

X. Uses

The present invention provides reagents which will find use in diagnostic applications as described elsewhere herein, e.g., in the general description for developmental abnormalities, or below in the description of kits for diagnosis.

An HCC5 chemokine; a Dub11 or Dub12 protein; or an MD1 or MD2 nucleotides, e.g., DNA or RNA, may be used as a component in a forensic assay. For instance, the nucleotide sequences provided may be labeled using, e.g., ³²P or biotin and used to probe standard restriction fragment polymorphism blots, providing a measurable character to aid in distinguishing between individuals or, e.g., species sources. Such probes may be used in well-known forensic techniques such as genetic fingerprinting. In addition, nucleotide probes made from HCC5; Dub11 or Dub12; and MD1 or MD2 sequences may be used in situ assays to detect chromosomal abnormalities. For instance, rearrangements in the human chromosome encoding an HCC5 chemokine gene; a Dub11 or Dub12 deubiquitinating gene; or an MD1 or MD2 gene may be detected via well-known in situ techniques, using HCC5 chemokine; Dub11 or Dub12 deubiquitinating; or MD1 or MD2 probes in conjunction with other known chromosome markers.

Antibodies and other binding agents directed towards HCC5 chemokine; a Dub11 or Dub12 protein; or an MD1 or MD2 proteins or nucleic acids may be used to purify a corresponding molecule. As described in the Examples below, antibody purification of HCC5 chemokine; Dub11 or Dub12 protein; or MD1 or MD2 components is both possible and practicable. Antibodies and other binding agents may also be used in a diagnostic fashion to determine whether an HCC5 chemokine; a Dub11 or Dub12; or an MD1 or MD2 component is present in a tissue sample or cell population using well-known techniques described herein. The ability to attach a binding agent to an HCC5 chemokine; a Dub11 or Dub12; or an MD1 or MD2 protein or nucleic acid provides a means to diagnose disorders associated with misregulation. Antibodies and other binding agents to a protein may also be useful as histological markers. As described in the examples below, HCC5; Dub11 or Dub12; or MD1 or MD2 expression are likely limited to specific tissue types. By directing an antibody or nucleic acid probe to an HCCS chemokine; Dub11 or Dub12; or MD1 or MD2 protein or nucleic acid it is possible to use the probe to distinguish tissue and cell types in situ or in vitro.

This invention also provides reagents with significant therapeutic value. The HCC5 chemokine (naturally occurring or recombinant), fragments thereof, and antibodies thereto, along with compounds identified as having binding affinity to a HCC5 chemokine, are useful in the treatment of conditions associated with abnormal physiology or development, including inflammatory conditions. Abnormal reactivity or inflammation may be modulated by appropriate therapeutic treatment using the compositions provided herein. For example, a disease or disorder associated with abnormal expression or abnormal signaling by a HCC5 chemokine is a target for an agonist or antagonist of the protein. The proteins likely play a role in regulation or development of hematopoietic cells, e.g., lymphoid cells, which affect immunological responses, or epithelial or neuronal cells.

Antagonists might be created by N-terminal modification, e.g., either truncation of addition of an N-terminal methionine. Since HCC5 is structurally related to the HCC chemokines, it may well exhibit similar behaviors and functions.

Other abnormal developmental conditions are known in cell types, e.g., breast, which possess HCC5 chemokine mRNA by sequence analysis. See Berkow (ed.) The Merck Manual of Diagnosis and Therapy, Merck & Co., Rahway, N.J.; and Thorn, et al. Harrison's Principles of Internal Medicine. McGraw-Hill, NY. Developmental or functional abnormalities, e.g., of the circulatory system, cause significant medical abnormalities and conditions which may be susceptible to prevention or treatment using compositions provided herein. Many chemokines have been demonstrated to exhibit angiostatic or angiogenic activities.

Certain chemokines have also been implicated in viral replication mechanisms, particularly the ligands for the CCR1, CCR3, and CCR5. See, e.g., Cohen (1996) Science 272:809-810; Feng, et al. (1996) Science 272:872-877; and Cocchi, et al. (1995) Science 270:1811-1816. The HCC5 chemokine may be useful in a similar context, alone or in combination with others. Because of the structural similarity to the other HCCs, it is likely that the HCC5 will bind to one or more of the receptors in the group or CCR1, CCR3, and CCR5. The HCC1 binds to CCR1 (Tsou, et al. (1998) J. Ex. Med. 188:603-608), and HCC2 binds to CCR1 and CCR3 (Youn, et al. (1997) J. Immunol. 159:5201-5205). The HCC3 is a splice variant of the HCC1, and probably will bind to CCR1. The HCC4 probably binds to a RANTES receptor, e.g., CCR1, CCR3, and/or CCR5. See Hedrick, et al. (1998) Blood 91:4242-4247. The HCC5 is likely to be expressed in selective tissues, e.g., breast, or only in specific developmental or physiological conditions.

The distribution of the HCC5 chemokines, especially in dendritic cells or in Th1 T cells, B cells, dendritic cells, and macrophages, suggest roles in immune functions, e.g., it will likely attract T cells and monocytes. Thus, the HCC5 chemokine is likely to recruit these cell types in vivo, thereby enhancing the immune response mediated by these cell types. The expression patterns appear consistent with a functional importance of the ligands in a TH1/TH2 regulation and/or response, including, e.g., in a cancer therapy. Thus, ligands and homologs are identified as possible immune adjuvants, e.g., for cellular responses, but also as possible adjuvants to modulate soluble antigen responses, e.g., vaccines.

Conversely, antagonists will have the opposite effects, and will be useful, e.g., in minimizing autoimmune or suppressing responses in desired contexts, e.g., in a tissue rejection situation. Antagonists may be muteins of the chemokine ligands, antibodies which block binding to receptor, or small drugs which interfere with the ligand-receptor interaction.

The Dub11 or Dub12 proteins (naturally occurring or recombinant), fragments thereof, and antibodies thereto, along with compounds identified as having binding affinity to Dub11 or Dub12, may be useful in the treatment of conditions associated with abnormal physiology or development, such as, e.g., uterine carcinoma associated with p53 dysregulation associated with human papilloma virus or mental retardation of Angelman syndrome (AS) due to disruption of the 5′ end of the UBE3A (E6-AP) gene which codes for a disubiquitination protein. Pharmacological intervention which alters the half-lives of cellular proteins associated with these diseases may have wide therapeutic potential. Specifically, prevention of p53 ubiquitination (and subsequent degradation) in human papilloma virus positive tumors, and perhaps all tumors retaining wild-type p53 but lacking the retinoblastoma gene function, could lead to programmed cell death. Additionally, specific inhibitors of p27 and cyclin B ubiquitination are predicted to be potent antiproliferative agents. Inhibitors of IkappaB ubiquitination should prevent NFkappaB activation and may have utility in a variety of autoimmune and inflammatory conditions. Finally, deubiquitination enzymes may be novel, potential drug targets as they also appear to regulate cell proliferation. These conditions or disease states may be modulated by appropriate therapeutic treatment using the deubiquitination compositions provided herein.

Conversely, methods for blocking the enzymatic activities should have the opposite effects. Small molecule drug screening to block enzymatic activity of the protein can be performed to identify entities which will block the deubiquitination, thereby affecting protein degradation pathways, as appropriate.

The T cell growth factor interleukin-2 (IL-2) regulates lymphocyte proliferation by inducing the expression of growth promoting genes. HTLV-1 transformed cell lines derived from Adult T-cell Leukemia (ATL) can exhibit constitutive activation of the IL-2-induced JAK/STAT pathway. See Migone, et al. (1998) Proc. Nat'l Acad. Sci. USA 95:3845-3850. ATL cell lines were examined for expression of IL-2 induced genes. It was found that the deubiquitinating enzyme Dub2 is constitutively expressed. See Zhu, et al. (1997) J. Biol. Chem. 272:51-57. Moreover, Dub2 expression conferred cytokine-independent proliferation on the interleukin-3-dependent murine Ba/F3 hematopoietic cell line. SCID mice (n=18) subcutaneously injected with Ba/F3 cells expressing Dub2, (but not a C to S inactive mutant of Dub2) developed tumors with a six week latency. Cells derived from these tumors exhibited constitutive tyrosine phosphorylation of STAT5 and also mimicked the ATL cell lines by exhibiting down-regulation of the protein tyrosine phosphatase SHP-1. These findings strongly indicate that Dub12 is an oncogene that, when constitutively expressed, can induce cytokine-independent growth in lymphocytes and may be implicated in leukemogenesis. It is likely that Dub2 controls cell growth by regulating the ubiquitin-dependent proteolysis or the ubiquitin-dependent state of a critical intracellular substrate. Functional similarity of the Dub11 and Dub12 would be expected. Thus, the biological role of Dub2 suggests similar important roles for the other Dub family members.

Screening for inhibitors of the Dub enzymes can also be easily accomplished using the known assays for activity. Such assays can be developed into high throughput screening efforts, testing, particularly, compounds known to affect protein turnover, or similar enzymatic sites. Small molecule antagonists of the enzymes, which will be membrane permeable, would be particularly desirable therapeutically.

The MD1 or MD2 proteins (naturally occurring or recombinant), fragments thereof, and antibodies thereto, along with compounds identified as having binding affinity to MD1 or MD2, should be useful in the treatment of conditions associated with abnormal physiology or development, such as, for example, the recognition of specific pathogenic molecules and the activation of B cell physiology. As indicated above, MD1 and MD2 exhibit structural motifs characteristic of ligands for the RP105 or BAS-1 surface receptors. Thus, soluble forms, antibodies, or small molecule drugs which disrupt intercellular signaling mediated by these receptors, will find use in modulating cellular response. These responses will be important in normal or abnormal clinical situations.

The HCC5, MD1, or MD2 (naturally occurring or recombinant), fragments thereof, and antibodies thereto, along with compounds identified as having binding affinity to them, should be useful in the treatment of conditions associated with abnormal physiology or development, including abnormal proliferation, e.g., cancerous conditions, or degenerative conditions. In particular, modulation of development of lymphoid cells will be achieved by appropriate therapeutic treatment using the compositions provided herein. For example, a disease or disorder associated with abnormal expression or abnormal signaling by a ligand or receptor should be a likely target for an agonist or antagonist of the antigen. The antigen plays a role in regulation or development of hematopoietic cells, e.g., lymphoid cells, which affect immunological responses, e.g., autoimmune disorders.

In particular, the antigen may provide a costimulatory signal to cell activation, or be involved in regulation of cell proliferation or differentiation. Thus, the HCC5, MD1, or MD2 will likely modulate cells which possess a receptor therefor, e.g., T cell mediated interactions with other cell types. These interactions would lead, in particular contexts, to modulation of cell growth, cytokine synthesis by those or other cells, or development of particular effector cells.

Moreover, the HCC5, MD1, or MD2 or antagonists could redirect T cell responses, e.g., between Th1 and Th2 polarization, or with Th0 cells, or may affect B cells or other lymphoid cell subsets. Among these agonists should be various antibodies which recognize the appropriate epitopes, e.g., which mimic binding of ligand or receptor to its partner. Alternatively, they may bind to epitopes which sterically can block receptor binding. Bone morphogenesis may be regulated by these receptor segments.

The ligands or receptors may provide a selective and powerful way to modulate immune responses in abnormal situations, e.g., autoimmune disorders, including rheumatoid arthritis, systemic lupus erythematosis (SLE), Hashimoto's autoimmune thyroiditis, as well as acute and chronic inflammatory responses in which T cell activation, expansion, and/or immunological T cell memory play an important role. See also Samter, et al. (eds.) Immunological Diseases vols. 1 and 2, Little, Brown and Co. Regulation of bone morphogenesis, T cell activation, expansion, and/or cytokine release by the naturally occurring secreted form of HCC5, MD1, or MD2, or an antagonist thereof, may be effected.

Recombinant HCC5 chemokine; Dub11 or Dub12 deubiquitinating; or MD1 or MD2 antibodies can be purified and then administered to a patient. These reagents can be combined for therapeutic use with additional active or inert ingredients, e.g., in conventional pharmaceutically acceptable carriers or diluents, e.g., immunogenic adjuvants, along with physiologically innocuous stabilizers and excipients. These combinations can be sterile filtered and placed into dosage forms as by lyophilization in dosage vials or storage in stabilized aqueous preparations. This invention also contemplates use of antibodies or binding fragments thereof, including forms which are not complement binding.

Drug screening using antibodies or receptor or fragments thereof can identify compounds having binding affinity to an HCC5 chemokine; a Dub11 or Dub12 protein; or an MD1 or MD2 protein, including isolation of associated components. Subsequent biological assays can then be utilized to determine if the compound has intrinsic stimulating activity and is therefore a blocker or antagonist in that it blocks the activity of the protein. Likewise, a compound having intrinsic stimulating activity can activate the receptor and is thus an agonist in that it simulates the activity of an HCC5 chemokine; a Dub11 or Dub12 protein; or an MD1 or MD2 protein . This invention further contemplates the therapeutic use of antibodies to an HCC5 chemokine; a Dub11 or Dub12 protein; or an MD1 or MD2 protein as antagonists.

The quantities of reagents necessary for effective therapy will depend upon many different factors, including means of administration, target site, physiological state of the patient, and other medicants administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman, et al. Goodman and Gilman's: The Pharmacological Bases of Therapeutics (current ed.) Pergamon Press; and Remington's Pharmaceutical Sciences (latest ed.) Mack Publishing Co., Easton, Pa. Methods for administration are discussed therein and below, e.g., for oral, intravenous, intraperitoneal, or intramuscular administration, transdermal diffusion, and others. Pharmaceutically acceptable carriers will include water, saline, buffers, and other compounds described, e.g., in the Merck Index, Merck & Co., Rahway, N.J. Dosage ranges would ordinarily be expected to be in amounts lower than 1 mM concentrations, typically less than about 10 μM concentrations, usually less than about 100 nM, preferably less than about 10 pM (picomolar), and most preferably less than about 1 fM (femtomolar), with an appropriate carrier. Slow release formulations, or a slow release apparatus will often be utilized for continuous administration. See, e.g., Langer (1990) Science 249:1527-1533.

Specific, or selective, antibodies can be purified and then administered to a patient, veterinary or human. These reagents can be combined for therapeutic use with additional active or inert ingredients, e.g., in conventional pharmaceutically acceptable carriers or diluents, e.g., immunogenic adjuvants, along with physiologically innocuous stabilizers, excipients, or preservatives. These combinations can be sterile filtered and placed into dosage forms as by lyophilization in dosage vials or storage in stabilized aqueous preparations. This invention also contemplates use of antibodies or binding fragments thereof, including forms which are not complement binding.

Another therapeutic approach included within the invention involves direct administration of reagents or compositions by any conventional administration techniques (e.g., but not restricted to local injection, inhalation, or administered systemically), to the subject with an immune, allergic, or trauma disorder. The reagents, formulations, or compositions included within the bounds and metes of the invention may also be targeted to specific cells by methods described herein. The actual dosage of reagent, formulation, or composition that modulates an immune, allergic, or trauma disorder depends on many factors, including the size and health of an organism, however one of ordinary skill in the art can use the following teachings describing the methods and techniques for determining clinical dosages. See, e.g., Spilker (1984) Guide to Clinical Studies and Developing Protocols, Raven Press Books, Ltd., New York, pp. 7-13, 54-60; Spilker (1991) Guide to Clinical Trials. Raven Press, Ltd., New York, pp. 93-101; Craig and Stitzel (eds. 1986) Modern Pharmacology, 2d ed., Little, Brown and Co., Boston, pp. 127-33; Speight (ed. 1987) Avery's Drug Treatment: Principles and Practice of Clinical Pharmacology and Therapeutics, 3d ed., Williams and Wilkins, Baltimore, pp. 50-56; Tallarida, et al. (1988) Principles in General Pharmacology, Springer-Verlag, New York, pp. 18-20). Generally, the dose will be in the range of about between 0.5 fg/ml and 500 μg/ml, inclusive, final concentration administered per day to an adult in a pharmaceutically acceptable carrier.

HCC5 chemokines; a Dub11 or Dub12 proteins; or an MD1 or MD2 proteins, fragments thereof, and antibodies to them or their fragments, antagonists, and agonists, may be administered directly to the host to be treated or, depending on the size of the compounds, it may be desirable to conjugate them to carrier proteins such as ovalbumin or serum albumin prior to their administration. Therapeutic formulations may be administered in many conventional dosage formulations, including sterile formulations. While it is possible for the active ingredient to be administered alone, it is preferable to present it as a pharmaceutical formulation. Formulations typically comprise at least one active ingredient, as defined above, together with one or more acceptable carriers thereof. Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy. See, e.g., Gilman, et al. (eds. 1990) Goodman and Gilman's: The Pharmacological Bases of Therapeutics (8th ed.) Pergamon Press; and (1990) Remington's Pharmaceutical Sciences (17th ed.) Mack Publishing Co., Easton, Pa.; Avis, et al. (eds. 1993) Pharmaceutical Dosage Forms: Parenteral Medications Dekker, NY; Lieberman, et al. (eds. 1990) Pharmaceutical Dosage Forms: Tablets Dekker, NY; and Lieberman, et al. (eds. 1990) Pharmaceutical Dosage Forms: Disperse Systems Dekker, NY. The therapy of this invention may be combined with or used in association with other therapeutic agents, e.g., with other similar reagents directed to the other chemokines, reagents which affect protein turnover, or MD intercellular signaling.

Both the naturally occurring and the recombinant forms of the HCC5 chemokine; the Dub11 or Dub12 protein; or the MD1 or MD2 proteins of this invention are particularly useful in kits and assay methods which are capable of screening compounds for binding activity to the proteins. Several methods of automating assays have been developed in recent years so as to permit screening of tens of thousands of compounds in a short period. See, e.g., Fodor, et al. (1991) Science 251:767-773, and other descriptions of chemical diversity libraries, which describe means for testing of binding affinity by a plurality of compounds. The development of suitable assays can be greatly facilitated by the availability of large amounts of purified, soluble HCC5 chemokine; Dub11 or Dub12; or an MD1 or MD2 as provided by this invention.

For example, antagonists can normally be found once the protein has been structurally defined. Testing of potential protein analogs is now possible upon the development of highly automated assay methods using a purified receptor. In particular, new agonists and antagonists will be discovered by using screening techniques described herein or well recognized in the art. Of particular importance are compounds found to have a combined binding affinity for multiple HCC5 chemokine; Dub11 or Dub12; or MD1 or MD2 binding partners, e.g., compounds which can serve as antagonists for species variants of an HCC5 chemokine; a Dub11 or Dub12 protein; or an MD1 or MD2 protein.

This invention is particularly useful for screening compounds by using recombinant protein in a variety of drug screening techniques. The advantages of using a recombinant protein in screening for specific ligands include: (a) improved renewable source of protein from a specific source; (b) potentially greater number of target proteins per cell giving better signal to noise ratio in assays; and (c) species variant specificity (theoretically giving greater biological and disease specificity).

One method of drug screening utilizes eukaryotic or prokaryotic host cells which are stably transformed with recombinant DNA molecules expressing the appropriate protein. Cells may be isolated which express, e.g., a protein in isolation from any others. Such cells, either in viable or fixed form, can be used for standard assays, e.g., in the case of the chemokine, ligand/receptor binding assays. See also, Parce, et al. (1989) Science 246:243-247; and Owicki, et al. (1990) Proc. Nat'l Acad. Sci. USA 87:4007-4011, which describe sensitive methods to detect cellular responses. Competitive assays are particularly useful, where the cells are contacted and incubated with a labeled antibody having known binding affinity to the ligand, such as ¹²⁵I-antibody, and a test sample whose binding affinity to the binding composition is being measured. The bound and free labeled binding compositions are then separated to assess the degree of ligand binding. The amount of test compound bound is inversely proportional to the amount of labeled receptor binding to the known source. Any one of numerous techniques can be used to separate bound from free ligand to assess the degree of ligand binding. This separation step could typically involve a procedure such as adhesion to filters followed by washing, adhesion to plastic followed by washing, or centrifugation of the cell membranes. Viable cells, or alternatively, in vitro assays, could also be used to screen for the effects of drugs on HCC5; Dub11 or Dub12; or MD1 or MD2 mediated functions. In the context of HCC5, e.g., second messenger levels, e.g., Ca⁺⁺; cell proliferation; inositol phosphate pool changes; etc., may be used. Some detection methods allow for elimination of a separation step, e.g., a proximity sensitive detection system. Calcium sensitive dyes will be useful for detecting Ca⁺⁺ levels, with a fluorimeter or a fluorescence cell sorting apparatus.

Another method utilizes membranes from transformed eukaryotic or prokaryotic host cells using the membranes as a source of HCC5; Dub11 or Dub12; or MD1 or MD2. These cells are stably transformed with DNA vectors directing the expression of HCC5; Dub11 or Dub12; or an MD1 or MD2, e.g., an engineered membrane bound form. Essentially, the membranes would be prepared from the cells and used, e.g., in the case of MD antigens, in a receptor/receptor binding assay, such as the competitive assay set forth above.

Still another approach is to use solubilized, unpurified or solubilized, purified protein from transformed eukaryotic or prokaryotic host cells. This allows for a “molecular” binding assay with the advantages of increased specificity, the ability to automate, and high drug test throughput.

Another technique for drug screening involves an approach which provides high throughput screening for compounds having suitable binding affinity to a protein, or antibody, and is described in detail in Geysen, European Patent Application 84/03564, published on Sep. 13, 1984. First, large numbers of different small peptide test compounds are synthesized on a solid substrate, e.g., plastic pins or some other appropriate surface, see Fodor, et al., supra. Then all the pins are reacted with solubilized, unpurified or solubilized, purified HCC5 chemokine antibody, and washed. The next step involves detecting bound HCC5; Dub11 or Dub12; or MD1 or MD2 antibody.

Rational drug design may also be based upon structural studies of the molecular shapes of the HCC5; Dub11 or Dub12; or MD1 or MD2 and other effectors or analogs. See, e.g., Methods in Enzymology vols. 202 and 203. In particular, the chemokine and surface antigens are feasible targets. Effectors may be other proteins which mediate other functions in response to ligand or receptor binding, or other proteins which normally interact with the receptor. One means for determining which sites interact with specific other proteins is a physical structure determination, e.g., x-ray crystallography or 2 dimensional NMR techniques. These will provide guidance as to which amino acid residues form molecular contact regions. For a detailed description of protein structural determination, see, e.g., Blundell and Johnson (1976) Protein Crystallography Academic Press, NY. These protein-protein interactions are subjects of protein structural analysis.

A purified HCC5 chemokine can be coated directly onto plates for use in the aforementioned drug screening techniques. However, non-neutralizing antibodies to these ligands can be used as capture antibodies to immobilize the respective ligand on the solid phase. Chemokine receptor screening methods are well known in the art, and the proposed chemokine-receptor pairings may be tested directly.

The matching of the MD and RP105 may also be easily tested. Identification of the counter receptor for the MD2 may include testing both the RP105 and BAS-1 genes, along with other screening methods, as described, e.g., below.

Screening for inhibitors of the Dub enzymes can also be easily accomplished using the known assays for activity. Such assays can be developed into high throughput screening efforts, testing, particularly, compounds known to affect protein turnover, or similar enzymatic sites.

XI. Kits

This invention also contemplates use of HCC5 chemokine proteins, fragments thereof, peptides, and their fusion products in a variety of diagnostic kits and methods for detecting the presence of protein or binding partner. Typically the kit will have a compartment containing either a defined HCC5; Dub11 or Dub12; or MD1 or MD2 peptide or gene segment or a reagent which recognizes one or the other, e.g., receptor fragments or antibodies. Alternatively, the kit may be developed to evaluate nucleic acid.

A kit for determining the binding affinity of a test compound to HCC5; Dub11 or Dub12; or MD1 or MD2 would typically comprise a test compound; a labeled compound, e.g., a binding partner such as a receptor or antibody having known binding affinity for HCC5; Dub11 or Dub12; or MD1 or MD2; a source of HCC5; Dub11 or Dub12; or MD1 or MD2 (naturally occurring or recombinant); and a means for separating bound from free labeled compound, such as a solid phase for immobilizing HCC5; Dub11 or Dub12; or MD1 or MD2. Once compounds are screened, those having suitable binding affinity to HCC5; Dub11 or Dub12; or MD1 or MD2 can be evaluated in suitable biological assays, as are well known in the art, to determine whether they act as agonists or antagonists to the receptor. The availability of recombinant HCC5; Dub11 or Dub12; or MD1 or MD2 polypeptides also provide well defined standards for calibrating such assays.

A preferred kit for determining the concentration of, e.g., HCC5; Dub11 or Dub12; or MD1 or MD2 in a sample would typically comprise a labeled compound, e.g., receptor or antibody, having known binding affinity for the HCC5; Dub11 or Dub12; or MD1 or MD2, a source of HCC5; Dub11 or Dub12; or MD1 or MD2(naturally occurring or recombinant), and a means for separating the bound from free labeled compound, for example, a solid phase for immobilizing HCC5; Dub11 or Dub12; or MD1 or MD2. Compartments containing reagents, and instructions, will normally be provided.

Antibodies, including antigen binding fragments, specific for HCC5; Dub11 or Dub12; or MD1 or MD2 or ligand fragments are useful in diagnostic applications to detect the presence of elevated levels of HCC5; Dub11 or Dub12; or MD1 or MD2 and/or its fragments. Such diagnostic assays can employ lysates, live cells, fixed cells, immunofluorescence, cell cultures, body fluids, and further can involve the detection of antigens related to the ligand in serum, or the like. Diagnostic assays may be homogeneous (without a separation step between free reagent and antigen-HCC5; Dub11 or Dub12; or MD1 or MD2 complex) or heterogeneous (with a separation step). Various commercial assays exist, such as radioimmunoassay (RIA), enzyme-linked immunosorbent assay (ELISA), enzyme immunoassay (EIA), enzyme-multiplied immunoassay technique (EMIT), substrate-labeled fluorescent immunoassay (SLFIA), and the like. For example, unlabeled antibodies can be employed by using a second antibody which is labeled and which recognizes the antibody to HCC5; Dub11 or Dub12; or MD1 or MD2 or to a particular fragment thereof. Similar assays have also been extensively discussed in the literature. See, e.g., Harlow and Lane (1988) Antibodies: A Laboratory Manual, CSH Press, NY; Chan (ed. 1987) Immunoassay: A Practical Guide Academic Press, Orlando, Fla.; Price and Newman (eds. 1991) Principles and Practice of Immunoassay Stockton Press, NY; and Ngo (ed. 1988) Non isotopic Immunoassay Plenum Press, NY.

Anti-idiotypic antibodies may have similar use to diagnose presence of antibodies against HCC5; Dub11 or Dub12; or MD1 or MD2, as such may be diagnostic of various abnormal states. For example, overproduction of HCC5 chemokine may result in production of various immunological or other medical reactions which may be diagnostic of abnormal physiological states, e.g., in cell growth, activation, or differentiation. Likewise for the Dub or MD proteins.

Frequently, the reagents for diagnostic assays are supplied in kits, so as to optimize the sensitivity of the assay. For the subject invention, depending upon the nature of the assay, the protocol, and the label, either labeled or unlabeled antibody or receptor, or labeled HCC5; Dub11 or Dub12; or MD1 or MD2 is provided. This is usually in conjunction with other additives, such as buffers, stabilizers, materials necessary for signal production such as substrates for enzymes, and the like. Preferably, the kit will also contain instructions for proper use and disposal of the contents after use. Typically the kit has compartments for each useful reagent. Desirably, the reagents are provided as a dry lyophilized powder, where the reagents may be reconstituted in an aqueous medium providing appropriate concentrations of reagents for performing the assay.

Many of the aforementioned constituents of the drug screening and the diagnostic assays may be used without modification, or may be modified in a variety of ways. For example, labeling may be achieved by covalently or non-covalently joining a moiety which directly or indirectly provides a detectable signal. In many of these assays, the protein, test compound, HCC5; Dub11 or Dub12; or MD1 or MD2, or antibodies thereto can be labeled either directly or indirectly. Possibilities for direct labeling include label groups: radiolabels such as ¹²⁵I, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups.

There are also numerous methods of separating the bound from the free ligand, or alternatively the bound from the free test compound. HCC5; Dub11 or Dub12; or MD1 or MD2 can be immobilized on various matrices followed by washing. Suitable matrices include plastic such as an ELISA plate, filters, and beads. Methods of immobilizing HCC5; Dub11 or Dub12; or MD1 or MD2. to a matrix include, without limitation, direct adhesion to plastic, use of a capture antibody, chemical coupling, and biotin-avidin. The last step in this approach involves the precipitation of ligand/receptor or ligand/antibody complex by any of several methods including those utilizing, e.g., an organic solvent such as polyethylene glycol or a salt such as ammonium sulfate. Other suitable separation techniques include, without limitation, the fluorescein antibody magnetizable particle method described in Rattle, et al. (1984) Clin. Chem. 30:1457-1461, and the double antibody magnetic particle separation as described in U.S. Pat. No. 4,659,678.

Methods for linking proteins or their fragments to the various labels have been extensively reported in the literature and do not require detailed discussion here. Many of the techniques involve the use of activated carboxyl groups either through the use of carbodiimide or active esters to form peptide bonds, the formation of thioethers by reaction of a mercapto group with an activated halogen such as chloroacetyl, or an activated olefin such as maleimide, for linkage, or the like. Fusion proteins will also find use in these applications.

Another diagnostic aspect of this invention involves use of oligonucleotide or polynucleotide sequences taken from the sequence of HCC5; Dub11 or Dub12; or MD1 or MD2 These sequences can be used as probes for detecting levels of the HCC5 chemokine message in samples from natural sources, or patients suspected of having an abnormal condition, e.g., cancer or developmental problem. The preparation of both RNA and DNA nucleotide sequences, the labeling of the sequences, and the preferred size of the sequences has received ample description and discussion in the literature. Normally an oligonucleotide probe should have at least about 14 nucleotides, usually at least about 18 nucleotides, and the polynucleotide probes may be up to several kilobases. Various labels may be employed, most commonly radionuclides, particularly ³²P However, other techniques may also be employed, such as using biotin modified nucleotides for introduction into a polynucleotide. The biotin then serves as the site for binding to avidin or antibodies, which may be labeled with a wide variety of labels, such as radionuclides, fluorophores, enzymes, or the like. Alternatively, antibodies may be employed which can recognize specific duplexes, including DNA duplexes, RNA duplexes, DNA-RNA hybrid duplexes, or DNA-protein duplexes. The antibodies in turn may be labeled and the assay carried out where the duplex is bound to a surface, so that upon the formation of duplex on the surface, the presence of antibody bound to the duplex can be detected. The use of probes to the novel anti-sense RNA may be carried out using many conventional techniques such as nucleic acid hybridization, plus and minus screening, recombinational probing, hybrid released translation (HRT), and hybrid arrested translation (HART). This also includes amplification techniques such as polymerase chain reaction (PCR).

Diagnostic kits which also test for the qualitative or quantitative presence of these and other markers are also contemplated. Diagnosis or prognosis may depend on the combination of multiple indications used as markers. Thus, kits may test for combinations of markers. See, e.g., Viallet, et al. (1989) Progress in Growth Factor Res. 1:89-97. Qualitative or quantitative expression of each chemokine may be evaluated by standard methods at the protein or mRNA levels.

XII. Binding Partner Isolation

Having isolated a binding partner of a specific interaction, methods exist for isolating the counter-partner. See, Gearing, et al. (1989) EMBO J. 8:3667-3676. For example, means to label HCC5; or MD1 or MD2 without interfering with the binding to its receptor can be determined. For example, an affinity label or epitope tag can be fused to either the amino- or carboxyl-terminus of the ligand. An expression library can be screened for specific binding of the HCC5 chemokine, e.g., by cell sorting, or other screening to detect subpopulations which express such a binding component. See, e.g., Ho, et al. (1993) Proc. Nat'l Acad. Sci. USA 90:11267-11271. Alternatively, a panning method may be used. See, e.g., Seed and Aruffo (1987) Proc. Nat'l Acad. Sci. USA 84:3365-3369. A two-hybrid selection system may also be applied making appropriate constructs with the available chemokine sequences. See, e.g., Fields and Song (1989) Nature 340:245-246. Standard Ca⁺⁺ flux methods can also be utilized. See, e.g., Coligan, et al. (eds. 1992 and periodic supplements) Current Protocols in Immunology Greene/Wiley, New York, N.Y.

Protein cross-linking techniques with label can be applied to isolate other binding partners of HCC5; Dub11 or Dub12; or MD1 or MD2. This would allow identification of proteins which specifically interact with HCC5; Dub11 or Dub12; or MD1 or MD2, e.g., in a ligand-receptor like manner. Typically, the chemokine family binds to receptors of the seven transmembrane receptor family, and the receptor for HCC5 is likely to exhibit a similar structure. In fact, it is likely that the HCC5 will bind to one or more of CCR1, CCR3, and CCR5. Thus, it is likely that the receptor will be found by expression in a system which is capable of expressing such a membrane protein in a form capable of exhibiting ligand binding capability.

The likely counter receptor structure for the MDs are RP105, BAS-1, and related genes. Associated proteins which bind to these, including the Dub proteins, may be identified using these techniques, among others.

Other methods can be used to determine the critical residues in the substrate, ligand, or receptor binding interactions. Mutational analysis can be performed, e.g., see Somoza, et al. (1993) J. Exp. Med. 178:549-558, to determine specific residues critical in the interaction and/or signaling. This will allow study of both extracellular domains, involved in the soluble ligand interaction, or intracellular domain of a transmembrane form, which provides interactions important in intracellular signaling.

For example, antagonists can normally be found once the antigen has been structurally defined, e.g., by tertiary structure data. Testing of potential interacting analogs is now possible upon the development of highly automated assay methods using a purified protein. In particular, new agonists and antagonists will be discovered by using screening techniques described herein. Of particular importance are compounds found to have a combined binding affinity for a spectrum of protein molecules, e.g., compounds which can serve as antagonists for species variants of the antigens.

The broad scope of this invention is best understood with reference to the following examples, which are not intended to limit the invention to specific embodiments.

### EXAMPLES

I. General Methods

Many of the standard methods below are described or referenced, e.g., in Maniatis, et al. (1982) Molecular Cloning, A Laboratory Manual Cold Spring Harbor Laboratory, Cold Spring Harbor Press, NY; Sambrook, et al. (1989) Molecular Cloning: A Laboratory Manual (2d ed.) Vols. 1-3, CSH Press, NY; Ausubel, et al. (1987 and Supplements) Current Protocols in Molecular Biology Wiley/Greene, NY; Innis, et al. (eds. 1990) PCR Protocols: A Guide to Methods and Applications Academic Press, NY; or Wells (1997) J. Leukoc. Biol. 61:545-550. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification,” Methods in Enzymology vol. 182, and other volumes in this series; and manufacturer's literature on use of protein purification products, e.g., Pharmacia, Piscataway, N.J., or Bio-Rad, Richmond, CA. Combination with recombinant techniques allow fusion to appropriate segments (epitope tags), e.g., to a FLAG sequence or an equivalent which can be fused, e.g., via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineerina, Principle and Methods 12:87-98, Plenum Press, NY; and Crowe, et al. (1992) QIAexpress: The High Level Expression & Protein Purification System QUIAGEN, Inc., Chatsworth, Calif.

Standard immunological techniques are described, e.g., in Hertzenberg, et al. (eds. 1996) Weir's Handbook of Experimental Immunology vols. 1-4, Blackwell Science; Coligan (1991) Current Protocols in Immunology Wiley/Greene, NY; and Methods in Enzymology volumes. 70, 73, 74, 84, 92, 93, 108, 116, 121, 132, 150, 162, and 163. Assays for neural cell biological activities are described, e.g., in Wouterlood (ed. 1995) Neuroscience Protocols modules 10, Elsevier; Methods in Neurosciences Academic Press; and Neuromethods Humana Press, Totowa, N.J. Methodology of developmental systems is described, e.g., in Meisami (ed.) Handbook of Human Growth and Developmental Biology CRC Press; and Chrispeels (ed.) Molecular Techniques and Approaches in Developmental Biology Interscience.

Computer sequence analysis is performed, e.g., using available software programs, including those from the GCG (U. Wisconsin) and GenBank sources. Public sequence databases were also used, e.g., from GenBank and others.

The FASTA (Pearson and Lipman, 1988) and BLAST (Altschul, et al. (1990) J. Mol. Biol. 215:403-410) programs were used to comb nonredundant protein and nucleotide databases (Benson, et al. (1994) Nucl. Acids Res. 22:3441-3444; Bairoch and Boeckmann (1994) Nucl. Acids Res. 22:3578-3580) with the resultant cDNA and encoded protein sequences. The sensitive search strategies of Altschul, et al. (1994) Nature Genet. 6:119-129; and Koonin, et al. (1994) EMBO J. 13:493-503; served as examples of how to locate distant structural homologues of protein chains. Multiple alignments of collected homologues were carried out with ClustalW (Thompson, et al. (1994) Comp. Applic. Biosci. 10:19-29) and MACAW (Schuler, et al. (1991) Proteins 9:180-190).

The membrane topologies of proteins, e.g., MD1, and a cohort of putative homologues were analyzed by a variety of methods that sought to determine the consensus number of domains, e.g., hydrophobic membrane-spanning helices and the likely cytoplasmic or extracellular exposure of the hydrophilic connecting loops. For single sequence analysis, the ALOM and MTOP (Klein, et al. (1985) Biochim. Biophys. Acta 815:468-476; and Hartmann, et al. (1989) Proc. Natl. Acad. Sci. USA 86:5786-5790) programs were accessed from the PSORT World-Wide Web site (Nakai and Kanehisa (1991) Proteins 11:95-110; and Nakai and Kanehisa (1992) Genomics 14:897-911); in turn, the TopPredII program (Claros and von Heijne (1994) Comp. Applic. Biosci. 10:685-686; MacIntosh PPC version) was used to parse chains into probable hydrophobic transmembrane regions, and further predict the localization by prevalence of charged residue types (von Heijne (1992) J. Mol. Biol. 225:487-494; and Sippos and von Heijne (1993) Eur. J. Biochem. 213:1333-1340). MEMSAT (Jones, et al. (1994) Biochem. 33:3038-3049; MS-DOS PC version) was likewise used to fit individual sequences into statistically-based topology models that render judgment on membrane spanning segments. Two Web-accessible programs that are able to make use of evolutionary data by analyzing multiply aligned sequences are PHD (Rost, et al. (1994) Comp. Applic. Biosci. 10:53-60; and Rost, et al. (1995) Protein Sci. 4:521-533) and TMAP (Persson and Argos (1994) J. Mol. Biol. 237:182-192); the former utilizes a neural network system to accurately predict the shared location of helical transmembrane segments in a protein family. Similar analysis of other proteins can be performed.

FACS analyses are described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

II. Isolation of Recombinant Clone

A clone encoding HCC5; Dub11 or Dub12; or MD1 or MD2 is isolated from a natural source by many different possible methods. Given the sequences provided herein, PCR primers or hybridization probes are selected and/or constructed to isolate either genomic DNA segments or cDNA reverse transcripts. Standard methods exist for extension of partial sequences. See, e.g., Sambrook, et al. or Coligan, et al.; Gibbons, et al. (1991) Proc. Nat'l Acad. Sci. USA 88:8563-8567; Parker, et al. (1991) Nucleic Acids Res. 19:3055-3060; Shyamala and Ames (1989) Gene 84:1-8; Verhasselt, et al. (1992) DNA Seq. 2:281-287; and Chiang, et al. (1995) BioTechniques 18:36. Genomic sequence analysis may be used, since the cell sources seem to be rare. Genetic and polymorphic or allelic variants are isolated by screening a population of individuals.

PCR based detection is performed by standard methods, preferably using primers from opposite ends of the coding sequence, but flanking segments might be selected for specific purposes.

Alternatively, hybridization probes are selected. Particular AT or GC contents of probes are considered depending upon the expected homology and mismatching expected. Appropriate stringency conditions are selected to balance an appropriate positive signal to background ratio. Successive washing steps are used to collect clones of greater homology.

Further clones are isolated using an antibody based selection procedure. Standard expression cloning methods are applied including, e.g., FACS staining of membrane associated expression product. The antibodies are used to identify clones producing a recognized protein. Alternatively, antibodies are used to purify HCC5; Dub11 or Dub12; or MD1 or MD2, with protein sequencing and standard means to isolate a gene encoding that protein.

Genomic sequence based methods will also allow for identification of sequences naturally available, or otherwise, which exhibit homology to the provided sequences.

III. Isolation of a Homologous Counterpart Clone

Similar methods are used as above to isolate an appropriate homologous genes. Similar source materials are used to isolate natural genes, including genetic, polymorphic, allelic, or strain variants. Other species variants are also isolated using similar methods. Alternatively, gene databases may be searched for the appropriate motifs.

Genes may be isolated from related primates, rodents, birds, etc. In particular, domestic animals and pets will be of interest, e.g., rodents, lagomorphs, carnivores, artiodactyla, perissodactyla, and primates.

IV. Chromosome Mapping by in situ Hybridization

Chromosome spreads are prepared. In situ hybridization is performed on chromosome preparations obtained from phytohemagglutinin-stimulated human lymphocytes cultured for 72 h. 5-bromodeoxyuridine was added for the final seven hours of culture (60 μg/ml of medium), to ensure a posthybridization chromosomal banding of good quality.

A PCR fragment, amplified with the help of primers, is cloned into an appropriate vector. The vector is labeled by nick-translation with ³H. The radiolabeled probe is hybridized to metaphase spreads at final concentration of 200 ng/ml of hybridization solution as described in Mattei, et al. (1985) Hum. Genet. 69:327-331.

After coating with nuclear track emulsion (KODAK NTB2), slides are exposed. To avoid any slipping of silver grains during the banding procedure, chromosome spreads are first stained with buffered Giemsa solution and metaphase photographed. R-banding is then performed by the fluorochrome-photolysis-Giemsa (FPG) method and metaphases re photographed before analysis.

Alternatively, FISH can be performed, or a BIOS Laboratories (New Haven, Conn.) mouse somatic cell hybrid panel may be combined with PCR methods. See Fan, et al. (1996) Immunogenetics 44:97-103.

Chromosome spreads are prepared. In situ hybridization is performed on chromosome preparations obtained from phytohemagglutinin-stimulated human lymphocytes cultured for 72 h. 5-bromodeoxyuridine is added for the final seven hours of culture (60 μg/ml of medium), to ensure a posthybridization chromosomal banding of good quality.

V. Expression; Purification; Characterization

With an appropriate clone from above, the coding sequence is inserted into an appropriate expression vector. This may be in a vector specifically selected for a prokaryote, yeast, insect, or higher vertebrate, e.g., mammalian expression system. Standard methods are applied to produce the gene product, preferably as a soluble secreted molecule, but will, in certain instances, also be made as an intracellular protein. Intracellular proteins typically require cell lysis to recover the protein, and insoluble inclusion bodies are a common starting material for further purification.

With a clone encoding, e.g., HCC5; Dub11 or Dub12; or MD1 or MD2, recombinant production means are used, although natural forms may be purified from appropriate sources. The protein product is purified by standard methods of protein purification, in certain cases, e.g., coupled with immunoaffinity methods. Immunoaffinity methods are used either as a purification step, typically small scale, as described above, or as a detection assay to determine the separation properties of the protein. Other larger scale purification methods may be used, with diagnosis of distribution evaluated by diagnostic reagents, e.g., antibody based methods.

Preferably, the protein is secreted into the medium, and the soluble product is purified from the medium in a soluble form. Alternatively, as described above, inclusion bodies from prokaryotic expression systems are a useful source of material. Typically, the insoluble protein is solubilized from the inclusion bodies and refolded using standard methods. Purification methods are developed as described above.

The product of the purification method described above is characterized to determine many structural features. Standard physical methods are applied, e.g,, amino acid analysis and protein sequencing. The resulting protein is subjected to CD spectroscopy and other spectroscopic methods, e.g., NMR, ESR, mass spectroscopy, etc. The product is characterized to determine its molecular form and size, e.g., using gel chromatography and similar techniques. Understanding of the chromatographic properties will lead to more gentle or efficient purification methods.

Prediction of glycosylation sites may be made, e.g., as reported in Hansen, et al. (1995) Biochem. J. 308:801-813. Alternatively, physical methods may be utilized to analyze purified samples.

A probe specific for cDNA encoding the subject gene is used to determine tissue distribution of message encoding the antigen. Standard hybridization probes may be used to do a Northern analysis of RNA from appropriate sources, either cells, e.g., stimulated, or in various physiological states, in various tissues, e.g., spleen, liver, thymus, lung, etc., or in various species. Southern analysis of cDNA libraries may also provide valuable distribution information. Standard tissue blots or species blots are commercially available. Similar techniques will be useful for evaluating diagnostic or medical conditions which may correlate with expression in various cell types.

PCR analysis using appropriate primers may also be used. Antibody analysis, including immunohistochemistry or FACS, may be used to determine cellular or tissue distribution.

Southern blot analysis of primate cDNA libraries is performed on, e.g.,: U937 premonocytic line, resting (MlOO); elutriated monocytes, activated with LPS, IFNY, anti-IL-10 for 4, 16 h pooled (M106); elutriated monocytes, activated with LPS, IFNγ, IL-10 for 4, 16 h pooled (M107); elutriated monocytes, activated LPS for 1 h (M108); elutriated monocytes, activated LPS for 6 h (M109); dendritic cells (DC) 30% CD1a+, from CD34+ GM-CSF, TNFα 12 days, resting; DC 70% CD1a+, from CD34+ GM-CSF, TNFα 12 days, resting (D101); DC 70% CD1a+, from CD34+ GM-CSF, TNFα 12 days, activated with PMA and ionomycin for 1 hr (D102); DC 70% CD1a+, from CD34+ GM-CSF, TNFα 12 days, activated with PMA and ionomycin for 6 hr (D103); DC 95% CD1a+, from CD34+ GM-CSF, TNFα 12 days activated with PMA and ionomycin for 1 or 6 hr, pooled; DC from monocytes GM-CSF, IL-4 5 days, resting (D107); DC from monocytes GM-CSF, IL-4 5 days, resting (D108); DC from monocytes GM-CSF, IL-4 5 days, activated TNFα, monocyte supe for 4, 16 h pooled (D110); EBV transfected B cell lines, resting; spleenocytes, resting; spleenocytes, activated with PMA and ionomycin; 20 NK clones resting, pooled; 20 NK clones activated with PMA and ionomycin, pooled; NKL clone, IL-2 treated; NK cytotoxic clone, resting; adipose tissue fetal 28 wk male (O108); brain fetal 28 wk male (O104); gallbladder fetal 28 wk male (O106); heart fetal 28 wk male (O103); small intestine fetal 28 wk male (O107); kidney fetal 28 wk male (O100); liver fetal 28 wk male (O102); lung fetal 28 wk male (O101); ovary fetal 25 wk female (O109); adult placenta 28 wk (O113); spleen fetal 28 wk male (O112); testes fetal 28 wk male (O111); uterus fetal 25 wk female (O110); TH0 clone Mot 72, resting (T102); T cell, TH0 clone Mot 72, activated with anti-CD28 and anti-CD3 for 3, 6, 12 h pooled (T103); T cell, TH0 clone Mot 72, anergic treated with specific peptide for 2, 7, 12 h pooled (T104); Th0 subtraction of resting from activated; T cell, TH1 clone HY06, resting (T107); T cell, TH1 clone HY06, activated with anti-CD28 and anti-CD3 for 3, 6, 12 h pooled (T108); T cell, TH1 clone HY06, anergic treated with specific peptide for 2, 6, 12 h pooled (T109); Th1 subtraction of resting from activated; T cell, TH2 clone HY935, resting (T110); T cell, TH2 clone HY935, activated with anti-CD28 and anti-CD3 for 2, 7, 12 h pooled (T111); and Th2 subtraction of resting from activated.

Samples for mouse mRNA distribution may include, e.g.,: resting mouse fibroblastic L cell line (C200); Braf:ER (Braf fusion to estrogen receptor) transfected cells, control (C201); T cells, TH1 polarized (Mel14 bright, CD4+ cells from spleen, polarized for 7 days with IFN-Y and anti IL-4; T200); T cells, TH2 polarized (Mel14 bright, CD4+ cells from spleen, polarized for 7 days with IL-4 and anti-IFN-γ; T201); T cells, highly TH1 polarized (see Openshaw, et al. (1995) J. Exp. Med. 182:1357-1367; activated with anti-CD3 for 2, 6, 16 h pooled; T202); T cells, highly TH2 polarized (see Openshaw, et al. (1995) J. Exp. Med. 182:1357-1367; activated with anti-CD3 for 2, 6, 16 h pooled; T203); CD44− CD25+ pre T cells, sorted from thymus (T204); TH1 T cell clone D1.1, resting for 3 weeks after last stimulation with antigen (T205); TH1 T cell clone D1.1, 10 μg/ml ConA stimulated 15 h (T206); TH2 T cell clone CDC35, resting for 3 weeks after last stimulation with antigen (T207); TH2 T cell clone CDC35, 10 μg/ml ConA stimulated 15 h (T208); Mel 14+ naive T cells from spleen, resting (T209); Mell4+ T cells, polarized to Th1 with IFN-γ/IL-12/anti-IL-4 for 6, 12, 24 h pooled (T210); Mel 14+ T cells, polarized to Th2 with IL-4/anti-IFN-γ for 6, 13, 24 h pooled (T211); unstimulated mature B cell leukemia cell line A20 (B200); unstimulated B cell line CH12 (B201); unstimulated large B cells from spleen (B202); B cells from total spleen, LPS activated (B203); metrizamide enriched dendritic cells from spleen, resting (D200); dendritic cells from bone marrow, resting (D201); monocyte cell line RAW 264.7 activated with LPS 4 h (M200); bone-marrow macrophages derived with GM and M-CSF (M201); macrophage cell line J774, resting (M202); macrophage cell line J774 +LPS +anti-IL-10 at 0.5, 1, 3, 6, 12 h pooled (M203); macrophage cell line J774+LPS+IL-10 at 0.5, 1, 3, 5, 12 h pooled (M204); aerosol challenged mouse lung tissue, Th2 primers, aerosol OVA challenge 7, 14, 23 h pooled (see Garlisi, et al. (1995) Clinical Immunology and Immunopathology 75:75-83; X206); Nippostrongulus-infected lung tissue (see Coffman, et al. (1989) Science 245:308-310; X200); total adult lung, normal (O200); total lung, rag-1 (see Schwarz, et al. (1993) Immunodeficiency 4:249-252; 0205); IL-10 K.O. spleen (see Kuhn, et al. (1991) Cell 75:263-274; X201); total adult spleen, normal (O201); total spleen, rag-1 (O207); IL-10 K.O. Peyer's patches (O202); total Peyer's patches, normal (O210); IL-10 K.O. mesenteric lymph nodes (X203); total mesenteric lymph nodes, normal (O211); IL-10 K.O. colon (X203); total colon, normal (O212); NOD mouse pancreas (see Makino, et al. (1980) Jikken Dobutsu 29:1-13; X205); total thymus, rag-1 (O208); total kidney, rag-1 (O209); total heart, rag-1 (O202); total brain, rag-1 (O203); total testes, rag-1 (O204); total liver, rag-1 (O206); rat normal joint tissue (O300); and rat arthritic joint tissue (X300).

A. Direct Protein Detection by Antibodies

Various cells, tissues, and developmental stages are stained with labeled antibodies. The detection may be immuno-histochemical for solid tissue, by FACS in disperse cells, and by other appropriate methods for other sample types. Antibodies specific for the various forms may be used to distinguish between membrane associated and soluble fragments. Various amplification means may be coupled to increase sensitivity.

B. Functional Detection

Specific neutralizing antibodies should provide means to specifically block the biological activity of the prostaglandin transporter. Activities related to prostaglandin binding, or to prostaglandin transport may be measured by sensitive means based upon knowledge of the normal biological function of the various forms.

Further testing of populations of cells, e.g., hematopoietic progenitors, or of other cell or tissue types will be useful to further determine distribution and likely function. Other tissue types, at defined developmental stages, and pathology samples may be screened to determine whether pathological states or stages may be advantageously correlated with the biological activity of the transporter.

VI. Preparation of Antibodies

With DNA for expression, or protein produced, e.g., as above, animals are immunized to produce antibodies. Polyclonal antiserum is raised, in some cases, using non-purified antigen, though the resulting serum will typically exhibit higher background levels. Preferably, the antigen is purified using standard protein purification techniques, including, e.g., affinity chromatography using polyclonal serum indicated above. Presence of specific antibodies is detected using defined synthetic peptide fragments.

Polyclonal serum is generally raised against a purified antigen, purified as indicated above, or using, e.g., a plurality of, synthetic peptides. A series of overlapping synthetic peptides which encompass all of the full length sequence, if presented to an animal, will produce serum recognizing most linear epitopes on the protein. Such an antiserum is used to affinity purify protein, which is, in turn, used to introduce intact full length protein into another animal to produce another antiserum preparation. Methods exist for immunoselecting, immunodepleting, or otherwise preferentially generating binding compositions which recognize either linear or conformational epitopes.

Preferred absorbent targets for HCC5 will be the HCC1, HCC2, HCC3, and HCC4 chemokines. Depleted preparations will show specificity, as appropriate for diagnostic or therapeutic purposes.

Similar techniques are used to generate monoclonal antibodies to either unpurified antigen, or, preferably, purified antigen. The antiserum or antibodies may recognize native protein, or may recognize denatured antigen.

VII. Cellular and Tissue Distribution

Distribution of the protein or gene products are determined, e.g., using immunohistochemistry with an antibody reagent, as produced above, or by screening for nucleic acids encoding the chemokine. Hybridization or PCR methods are used to detect DNA, cDNA, or message content. Histochemistry allows determination of the specific cell types within a tissue which express higher or lower levels of message or DNA. Antibody techniques are useful to quantitate protein in a biological sample, including a liquid or tissue sample. Immunoassays are developed to quantitate protein. Also, FACS analysis may be used to evaluate expression in a cell population.

Southern Analysis: DNA (5 μg) from a primary amplified cDNA library is digested with appropriate restriction enzymes to release the inserts, run on a 1% agarose gel and transferred to a nylon membrane (Schleicher and Schuell, Keene, N.H.).

Samples for human mRNA isolation would typically include, e.g.: peripheral blood mononuclear cells (monocytes, T cells, NK cells, granulocytes, B cells), resting (T100); peripheral blood mononuclear cells, activated with anti-CD3 for 2, 6, 12 h pooled (T101); T cell, TH0 clone Mot 72, resting (T102); T cell, TH0 clone Mot 72, activated with anti-CD28 and anti-CD3 for 3, 6, 12 h pooled (T103); T cell, TH0 clone Mot 72, anergic treated with specific peptide for 2, 7, 12 h pooled (T104); T cell, TH1 clone HY06, resting (T107); T cell, TH1 clone HY06, activated with anti-CD28 and anti-CD3 for 3, 6, 12 h pooled (T108); T cell, TH1 clone HY06, anergic treated with specific peptide for 2, 6, 12 h pooled (T109); T cell, TH2 clone HY935, resting (T110); T cell, TH2 clone HY935, activated with anti-CD28 and anti-CD3 for 2, 7, 12 h pooled (T111); T cells CD4+CD45RO− T cells polarized 27 days in anti-CD28, IL-4, and anti IFN-γ, TH2 polarized, activated with anti-CD3 and anti-CD28 4 h (T116); T cell tumor lines Jurkat and Hut78, resting (T117); T cell clones, pooled AD130.2, Tc783.12, Tc783.13, Tc783.58, Tc782.69, resting (T118); T cell random γδ T cell clones, resting (T119); Splenocytes, resting (B100); Splenocytes, activated with anti-CD40 and IL-4 (B100); B cell EBV lines pooled WT49, RSB, JY, CVIR, 721.221, RM3, HSY, resting (B102); B cell line JY, activated with PMA and ionomycin for 1, 6 h pooled (B103); NK 20 clones pooled, resting (KlOO); NK 20 clones pooled, activated with PMA and ionomycin for 6 h (K101); NKL clone, derived from peripheral blood of LGL leukemia patient, IL-2 treated (K106); NK cytotoxic clone 640-A30-1, resting (K107); hematopoietic precursor line TF1, activated with PMA and ionomycin for 1, 6 h pooled (C100); U937 premonocytic line, resting (M100); U937 premonocytic line, activated with PMA and ionomycin for 1, 6 h pooled (M101); elutriated monocytes, activated with LPS, IFNγ, anti-IL-10 for 1, 2, 6, 12, 24 h pooled (M102); elutriated monocytes, activated with LPS, IFNγ, IL-10 for 1, 2, 6, 12, 24 h pooled (M103); elutriated monocytes, activated with LPS, IFNγ, anti-IL-10 for 4, 16 h pooled (M106); elutriated monocytes, activated with LPS, IFNγ, IL-10 for 4, 16 h pooled (M107); elutriated monocytes, activated LPS for 1 h (M108); elutriated monocytes, activated LPS for 6 h (M109); DC 70% CD1a+, from CD34+ GM-CSF, TNFα 12 days, resting (D101); DC 70% CD1a+, from CD34+ GM-CSF, TNFα 12 days, activated with PMA and ionomycin for 1 hr (D102); DC 70% CD1a+, from CD34+ GM-CSF, TNFα 12 days, activated with PMA and ionomycin for 6 hr (D103); DC 95% CD1a+, from CD34+ GM-CSF, TNFα 12 days FACS sorted, activated with PMA and ionomycin for 1, 6 h pooled (D104); DC 95% CD14+, ex CD34+ GM-CSF, TNFα 12 days FACS sorted, activated with PMA and ionomycin 1, 6 hr pooled (D105); DC CD1a+CD86+, from CD34+ GM-CSF, TNFα 12 days FACS sorted, activated with PMA and ionomycin for 1, 6 h pooled (D106); DC from monocytes GM-CSF, IL-4 5 days, resting (D107); DC from monocytes GM-CSF, IL-4 5 days, resting (D108); DC from monocytes GM-CSF, IL-4 5 days, activated LPS 4, 16 h pooled (D109); DC from monocytes GM-CSF, IL-4 5 days, activated TNFa, monocyte supe for 4, 16 h pooled (D110); leiomyoma L11 benign tumor (X101); normal myometrium M5 (O115); malignant leiomyosarcoma GS1 (X103); lung fibroblast sarcoma line MRC5, activated with PMA and ionomycin for 1, 6 h pooled (C101); kidney epithelial carcinoma cell line CHA, activated with PMA and ionomycin for 1, 6 h pooled (C102); kidney fetal 28 wk male (O100); lung fetal 28 wk male (O101); liver fetal 28 wk male (O102); heart fetal 28 wk male (O103); brain fetal 28 wk male (O104); gallbladder fetal 28 wk male (O106); small intestine fetal 28 wk male (O107); adipose tissue fetal 28 wk male (O108); ovary fetal 25 wk female (O109); uterus fetal 25 wk female (O110); testes fetal 28 wk male (O111); spleen fetal 28 wk male (O112); adult placenta 28 wk (O113); and tonsil inflamed, from 12 year old (X100).

Samples for mouse mRNA isolation can include, e.g.: resting mouse fibroblastic L cell line (C200); Braf:ER (Braf fusion to estrogen receptor) transfected cells, control (C201); T cells, TH1 polarized (Mell4 bright, CD4+ cells from spleen, polarized for 7 days with IFN-y and anti IL-4; T200); T cells, TH2 polarized (Mell4 bright, CD4+ cells from spleen, polarized for 7 days with IL-4 and anti-IFN-γ; T201); T cells, highly TH1 polarized (see Openshaw, et al. (1995) J. Exp. Med. 182:1357-1367; activated with anti-CD3 for 2, 6, 16 h pooled; T202); T cells, highly TH2 polarized (see Openshaw, et al. (1995) J. Exp. Med. 182:1357-1367; activated with anti-CD3 for 2, 6, 16 h pooled; T203); CD44− CD25+ pre T cells, sorted from thymus (T204); TH1 T cell clone D1.1, resting for 3 weeks after last stimulation with antigen (T205); TH1 T cell clone D1.1, 10 μg/ml ConA stimulated 15 h (T206); TH2 T cell clone CDC35, resting for 3 weeks after last stimulation with antigen (T207); TH2 T cell clone CDC35, 10 μg/ml ConA stimulated 15 h (T208); Mell4+ naive T cells from spleen, resting (T209); Mell4+ T cells, polarized to Th1 with IFN-γ/IL-12/anti-IL-4 for 6, 12, 24 h pooled (T210); Mel14+ T cells, polarized to Th2 with IL-4/anti-IFN-7 for 6, 13, 24 h pooled (T211); unstimulated mature B cell leukemia cell line A20 (B200); unstimulated B cell line CH12 (B201); unstimulated large B cells from spleen (B202); B cells from total spleen, LPS activated (B203); metrizamide enriched dendritic cells from spleen, resting (D200); dendritic cells from bone marrow, resting (D201); monocyte cell line RAW 264.7 activated with LPS 4 h (M200); bone-marrow macrophages derived with GM and M-CSF (M201); macrophage cell line J774, resting (M202); macrophage cell line J774+LPS+anti-IL-10 at 0.5, 1, 3, 6, 12 h pooled (M203); macrophage cell line J774+LPS+IL-10 at 0.5, 1, 3, 5, 12 h pooled (M204); aerosol challenged mouse lung tissue, Th2 primers, aerosol OVA challenge 7, 14, 23 h pooled (see Garlisi, et al. (1995) Clinical Immunology and Immunopathology 75:75-83; X206); Nippostrongulus-infected lung tissue (see Coffman, et al. (1989) Science 245:308-310; X200); total adult lung, normal (O200); total lung, rag-l (see Schwarz, et al. (1993) Immunodeficiency 4:249-252; 0205); IL-10 K.O. spleen (see Kuhn, et al. (1991) Cell 75:263-274; X201); total adult spleen, normal (O201); total spleen, rag-1 (O207); IL-10 K.O. Peyer's patches (O202); total Peyer's patches, normal (O210); IL-10 K.O. mesenteric lymph nodes (X203); total mesenteric lymph nodes, normal (O211); IL-10 K.O. colon (X203); total colon, normal (O212); NOD mouse pancreas (see Makino, et al. (1980) Jikken Dobutsu 29:1-13; X205); total thymus, rag-1 (O208); total kidney, rag-1 (O209); total heart, rag-l (O202); total brain, rag-l (O203); total testes, rag-1 (O204); total liver, rag-l (O206); rat normal joint tissue (O300); and rat arthritic joint tissue (X300).

VIII. Protein Expression

PCR is used to make a construct comprising the open reading frame, preferably in operable association with proper promoter, selection, and regulatory sequences. The resulting expression plasmid is transformed into an appropriate cell type, e.g., the Topp5, E. coli strain (Stratagene, La Jolla, Calif.). Ampicillin resistant (50 μg/ml) transformants are grown in Luria Broth (Gibco) at 37° C. until the optical density at 550 nm is 0.7. Recombinant protein is induced with-0.4 mM isopropyl-βD-thiogalacto-pyranoside (Sigma, St. Louis, Mo.) and incubation of the cells continued at 20° C. for a further 18 hours. Cells from a 1 liter culture are harvested by centrifugation and resuspended, e.g., in 200 ml of ice cold 30% sucrose, 50 mM Tris HCl pH 8.0, 1 mM ethylenediaminetetraacetic acid. After 10 min on ice, ice cold water is added to a total volume of 2 liters. After 20 min on ice, cells are removed by centrifugation and the supernatant is clarified by filtration via a 5 μM Millipak 60 (Millipore Corp., Bedford, Mass.).

The recombinant protein is purified via standard purification methods, e.g., various ion exchange chromatography methods. Immunoaffinity methods using antibodies described below can also be used. Affinity methods may be used where an epitope tag is engineered into an expression construct.

Similar methods are used to prepare expression constructs and cells in eukaryotic cells. Eukaryotic promoters and expression vectors may be produced, as described above.

Further study of the expression and control of the subject gene will be pursued. The controlling elements associated with the antigens may exhibit differential developmental, tissue specific, or other expression patterns. Upstream or downstream genetic regions, e.g., control elements, are of interest.

Multiple transfected cell lines are screened for one which expresses the antigen, membrane bound, or soluble forms, at a high level compared with other cells. Various cell lines are screened and selected for their favorable properties in handling. Natural protein can be isolated from natural sources, or by expression from a transformed cell using an appropriate expression vector. Purification of the expressed protein is achieved by standard procedures, or may be combined with engineered means for effective purification at high efficiency from cell lysates or supernatants. FLAG or His6 segments can be used for such purification features.

The gene product is isolated by a combination of affinity chromatography using the prostaglandin transporter specific binding compositions, e.g., antibody, as a specific binding reagent in combination with protein purification techniques allowing separation from other proteins and contaminants. Various detergent combinations may be tested to determine what combinations will retain biological activity while solubilizing contaminants. The purification may follow biological activity, e.g., prostaglandin binding or transport into membranes, or by ELISA or other structural binding reagents.

Similar methods are applied for purification of other polypeptides.

IX. Chemotaxis Assays

Chemokine proteins are produced, e.g., in COS cells transfected with a plasmid carrying the chemokine cDNA by electroporation. See, Hara, et al. (1992) EMBO J. 10:1875-1884. Physical analytical methods may be applied, e.g., CD analysis, to compare tertiary structure to other chemokines to evaluate whether the protein has likely folded into an active conformation. After transfection, a culture supernatant is collected and subjected to bioassays. A mock control, e.g., a plasmid carrying the luciferase cDNA, is used. See, de Wet, et al. (1987) Mol. Cell. Biol. 7:725-757. A positive control, e.g., recombinant murine MIP-1α from R&D Systems (Minneapolis, Minn.), is typically used. Likewise, antibodies may be used to block the biological activities, e.g., as a control.

Lymphocyte migration assays are performed as previously described, e.g., in Bacon, et al. (1988) Br. J. Pharmacol. 95:966-974. Other trafficking assays are also available. See, e.g., Quidling-Järbrink, et al. (1995) Eur. J. Immunol. 25:322-327; Koch, et al. (1994) J. Clinical Investigation 93:921-928; and Antony, et al. (1993) J. Immunol. 151:7216-7223. Murine Th2 T cell clones, CDC-25 (see Tony, et al. (1985) J. Exp. Med. 161:223-241) and HDK-1 (see Cherwinski, et al. (1987) J. Exp. Med. 166:1229-1244), made available from R. Coffman and A. O'Garra (DNAX, Palo Alto, Calif.), respectively, are used as controls.

Ca2+ flux upon chemokine stimulation is measured according to the published procedure described in Bacon, et al. (1995) J. Immunol. 154:3654-3666.

Maximal numbers of migrating cells in response to MIP-1α typically occur at a concentration of 10-8 M, in agreement with original reports for CD4+ populations of human T cells. See Schall (1993) J. Exp. Med. 177:1821-1826. A dose-response curve is determined, preferably giving a characteristic bell shaped dose-response curve.

After stimulation with CC chemokines, lymphocytes generally show a measurable intracellular Ca2+ flux. MIP-1α is capable of inducing immediate transients of calcium mobilization. Typically, the levels of chemokine used in these assays will be comparable to those used for the chemotaxis assays (1/1000 dilution of conditioned supernatants).

X. Biological Activities, Direct and Indirect

A. Chemokine

A robust and sensitive assay is selected as described above, e.g., on immune cells, neuronal cells, or stem cells. Chemokine is added to the assay in increasing doses to see if a dose response is detected. For example, in a proliferation assay, cells are plated out in plates. Appropriate culture medium is provided, and chemokine is added to the cells in varying amounts. Growth is monitored over a period of time which will detect either a direct effect on the cells, or an indirect effect of the chemokine.

Alternatively, an activation assay or attraction assay is used. An appropriate cell type is selected, e.g., hematopoietic cells, myeloid (macrophages, neutrophils, polymorphonuclear cells, etc.) or lymphoid (T cell, B cell, or NK cells), neural cells (neurons, neuroglia, oligodendrocytes, astrocytes, etc.), or stem cells, e.g., progenitor cells which differentiate to other cell types, e.g., gut crypt cells and undifferentiated cell types.

Retroviral infection assays have also been described using, e.g., the CCR1, CCR3, and CCR5 receptors. These receptors, which bind the RANTES and MIP-1 related chemokines, are likely also to be receptors for the HCC5 Recent description of these chemokine receptors in retroviral infection processes, and the effects by the related RANTES and MIP-1 chemokines, suggest similar effects may exist with the HCC5. See, e.g., Balter (1996) Science 272:1740 (describing evidence for chemokine receptors as coreceptors for HIV); and Deng, et al. (1996) Nature 381:661-666.

Chemokines may also be assayed for activity in hemopoietic assays as described, e.g., by H. Broxmeyer. See Bellido, et al. (1995) J. Clinical Investigation 95:2886-2895; and Jilka, et al. (1995) Expt'l Hematology 23:500-506. They may be assayed for angiogenic activities as described, e.g., by Streiter, et al. (1992) Am. J. Pathol. 141:1279-1284. Or for a role in inflammation. See, e.g., Wakefield, et al. (1996) J. Surgical Res. 64:26-31.

Other assays will include those which have been demonstrated with other chemokines. See, e.g., Schall and Bacon (1994) Current Opinion in Immunology 6:865-873; and Bacon and Schall (1996) Int. Arch. Allergy & Immunol. 109:97-109.

B. Dubs

The Dub genes will be screened for the deubiquitinating activities, as described. See, e.g., Hochstrasser (1995) Curr. Opin. Cell Biol. 7:215-223; Wilkinson, et al. (1995) Biochemistry 34:14535-14546; Baker, et al. (1992) J. Biol. Chem. 267:23364-23375; Baek et al. (1998) J. Biol. Chem. 272:(41) 25560-25565; and Papa and Hochstrasser (1993) Nature 366:313-319. For example, for an in vitro assay for UBP Activity, ¹²⁵I-labeled Ub-PESTc is used as a substrate according to the method of Woo, et al. (1995) J. Biol. Chem. 270:18766-18773. Reaction mixtures, e.g., 0.1 ml, contain the proper amount of the enzyme preparations and 10-30 μg of ¹²⁵I-labeled Ub-PESTC in 100 mM Tris-HCl (pH 7.8), 1 mM dithiothreitol, 1 mM EDTA, and 5% glycerol. After incubating the mixtures for appropriate periods, the reaction is terminated, e.g., by adding 50 μl of 40% (w/v) trichloroacetic acid and 50 μl of 1.2% (w/v) bovine serum albumin. The samples are centrifuged, and the resulting supernatants are counted for their radioactivities using a y-counter. The enzyme activity is expressed as a percentage of ¹²⁵I-labeled Ub-PESTc hydrolyzed to acid-soluble products. When assaying the hydrolysis of Ub-NH-carboxyl extension proteins and His-di-Ub, incubations are performed as above but in the presence of 5 μg of the substrate. After incubation for appropriate periods, the samples are subjected to discontinuous gel electrophoresis, e.g., as described by Baek, et al. (1998) J. Biol. Chem. 272:25560-25565. Proteins in the gels were then visualized, e.g., by staining with Coomassie Blue R-250 or by exposing to x-ray films (Fuji) at −700 C. To prepare ¹²⁵I-labeled poly-Ub-NH-lysozyme conjugates, 2 μg of the ¹²⁵I-labeled lysozyme (5×10⁵ cpm) are incubated with 10 μg of Ub, 120 μg of fraction II, and an ATP-regenerating system consisting of 10 mM Tris-HCl (pH 7.8), 15 units/ml creatine phosphokinase, 6.5 mM phosphocreatine, 1.5 mM ATP, 1 mM dithiothreitol, 0.5 mM MgCl₂, and 1 mM KCl in a final volume of 0.05 ml. Incubations are performed for 2 h at 37° C. in the presence of 1 mM hemin to prevent proteolysis of the ubiquitinated protein conjugates by the 26S proteasome. After incubation, the samples are heated for 10 min at 55° C. for inactivation of endogenous UBPs. Alternatively, Dub11 or Dub12 can be expressed as a GST fusion protein according to the method of Zhu, et al. (1997) J. Biol. Chem. 272:51-57 by cloning into an appropriate expression vector and subsequently co-transformed with a plasmid encoding Ub-Met-β-gal, in which ubiquitin is fused to the NH2 terminus of β-galactosidase and testing for cleavage.

However, the deubiquitinating enzymes have also been reported to have additional functions besides deubiquitination. See, e.g., Hochstrasser (1996) Cell 84:813-815; Hicke and Riezman (1996) Cell 84:277-287; and Chen, et al. (1996) Cell 84:853-862.

C. MDs

The MD gene products will be screened for cell signaling activities. See, e.g., Miyake, et al. (1998) J. Immunol. 161:1348-1353; and Kobe and Deisenhofer (1994) Trends Biochem. Sci. 19:412.

XI. Structure Activity Relationship

Information on the criticality of particular residues is determined using standard procedures and analysis. Standard mutagenesis analysis is performed, e.g., by generating many different variants at determined positions, e.g., at the positions identified above, and evaluating biological activities of the variants. This may be performed to the extent of determining positions which modify activity, or to focus on specific positions to determine the residues which can be substituted to either retain, block, or modulate biological activity.

Alternatively, analysis of natural variants can indicate what positions tolerate natural mutations. This may result from populational analysis of variation among individuals, or across strains or species. Samples from selected individuals are analyzed, e.g., by PCR analysis and sequencing. This allows evaluation of population polymorphisms.

XII. Screening for Agonists/Antagonists

Agonists or antagonists are screened for ability to induce or block biological activity. The candidate compounds, e.g., sequence variants of HCC5; or MD1 or MD2, are assayed for interaction with putative receptors, typically effecting biological activities. Alternatively, compounds are screened, alone or in combinations, to determine effects on biological activity. Small molecule drugs, or antibodies may be screened for antagonizing, or neutralizing capability.

XIII. Identification or Isolation of a Receptors

HCC5; or MD1 or MD2 can be used as a specific binding reagent to identify its binding partner, by taking advantage of its specificity of binding, much like an antibody would be used. A binding reagent is either labeled as described above, e.g., fluorescence or otherwise, or immobilized to a substrate for panning methods. The typical chemokine receptor is a seven transmembrane receptor. As indicated above, the CCR1, CCR3, and CCR5 receptors would be the first candidates for testing, then the other CCRs. However, functional testing may be preferred over some of the following methods.

The binding composition, e.g., chemokine, is used to screen an expression library made from a cell line which expresses a binding partner, i.e., receptor. Standard staining techniques are used to detect or sort intracellular or surface expressed receptor, or surface expressing transformed cells are screened by panning. Screening of intracellular expression is performed by various staining or immunofluorescence procedures. See also McMahan, et al. (1991) EMBO J. 10:2821-2832.

Standard Ca⁺⁺ flux protocols, see, e.g., Coligan, et al. (eds.) (1992 and periodic supplements) Current Protocols in Immunol. Greene/Wiley, New York, N.Y., can be used to identify a receptor for HCC5.

Alternatively, methods are available to identify receptors, e.g., of the MD gene products. For example, on day 0, precoat 2-chamber permanox slides with 1 ml per chamber of fibronectin, 10 ng/ml in PBS, for 30 min at room temperature. Rinse once with PBS. Then plate COS cells at 2-3×10⁵ cells per chamber in 1.5 ml of growth media. Incubate overnight at 37° C.

On day 1 for each sample, prepare 0.5 ml of a solution of 66 μg/ml DEAE-dextran, 66 μM chloroquine, and 4 μg DNA in serum free DME. For each set, a positive control is prepared, e.g., of human HCC5 chemokine cDNA at 1 and 1/200 dilution, and a negative mock. Rinse cells with serum free DME. Add the DNA solution and incubate 5 hr at 37° C. Remove the medium and add 0.5 ml 10% DMSO in DME for 2.5 min. Remove and wash once with DME. Add 1.5 ml growth medium and incubate overnight.

On day 2, change the medium. On days 3 or 4, the cells are fixed and stained. Rinse the cells twice with Hank's Buffered Saline Solution (HBSS) and fix in 4% paraformaldehyde (PFA)/glucose for 5 min. Wash 3× with HBSS. The slides may be stored at −80° C. after all liquid is removed. For each chamber, 0.5 ml incubations are performed as follows. Add HBSS/saponin (0.1%) with 32 μl/ml of 1 M NaN₃ for 20 min. Cells are then washed with HBSS/saponin 1×. Add chemokine or chemokine/antibody complex to cells and incubate for 30 min. Wash cells twice with HESS/saponin. If appropriate, add first antibody for 30 min. Add second antibody, e.g., Vector anti-mouse antibody, at 1/200 dilution, and incubate for 30 min. Prepare ELISA solution, e.g., Vector Elite ABC horseradish peroxidase solution, and preincubate for 30 min. Use, e.g., 1 drop of solution A (avidin) and 1 drop solution B (biotin) per 2.5 ml HBSS/saponin. Wash cells twice with HBSS/saponin. Add ABC HRP solution and incubate for 30 min. Wash cells twice with HBSS, second wash for 2 min, which closes cells. Then add Vector diaminobenzoic acid (DAB) for 5 to 10 min. Use 2 drops of buffer plus 4 drops DAB plus 2 drops of H₂O₂ per 5 ml of glass distilled water. Carefully remove chamber and rinse slide in water. Air dry for a few minutes, then add 1 drop of Crystal Mount and a cover slip. Bake for 5 min at 85-90 C.

Evaluate positive staining of pools and progressively subclone to isolation of single genes responsible for the binding.

Alternatively, chemokine reagents are used to affinity purify or sort out cells expressing a receptor. See, e.g., Sambrook, et al. or Ausubel, et al.

Another strategy is to screen for a membrane bound receptor by panning. The receptor cDNA is constructed as described above. The ligand can be immobilized and used to immobilize expressing cells. Immobilization may be achieved by use of appropriate antibodies which recognize, e.g., a FLAG sequence of a chemokine fusion construct, or by use of antibodies raised against the first antibodies. Recursive cycles of selection and amplification lead to enrichment of appropriate clones and eventual isolation of receptor expressing clones.

Phage expression libraries can be screened by chemokine. Appropriate label techniques, e.g., anti-FLAG antibodies, will allow specific labeling of appropriate clones.

XIV. Immunohistochemical Localization

The antibodies described above are used to identify expression of HCC5; Dub11 or Dub12; or MD1 or MD2 in various tissues or samples. Methods for immunohistochemical staining are described, e.g., in Sheehan, et al. (eds. 1987) Theory and Practice of Histotechnology, Battelle Press, Columbus, Ohio.

All references cited herein are incorporated herein by reference to the same extent as if each individual publication or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A composition of matter selected from:
a) a substantially pure or recombinant HCC5 polypeptide exhibiting identity over a length of at least 12 amino acids to SEQ ID NO: 2;
b) an isolated natural sequence HCC5 of mature SEQ ID NO: 2;
c) a fusion protein comprising HCC5 sequence;
d) a substantially pure or recombinant Dub11 polypeptide exhibiting identity over a length of at least about 12 amino acids to SEQ ID NO: 9 or 11;
e) an isolated natural sequence Dub11 of mature SEQ ID NO: 9 or 11;
f) a fusion protein comprising Dub11 sequence;
g) a substantially pure or recombinant Dub12 polypeptide exhibiting identity over a length of at least about 12 amino acids to SEQ ID NO: 13 or 15;
h) an isolated natural sequence Dub12 of mature SEQ ID NO: 13 or 15;
i) a fusion protein comprising Dub12 sequence;
j) a substantially pure or recombinant MD1 polypeptide exhibiting identity over a length of at least about 12 amino acids to SEQ ID NO: 19;
k) an isolated natural sequence MD1 of mature SEQ ID NO: 19;
l) a fusion protein comprising primate MD1 sequence;
m) a substantially pure or recombinant MD2 polypeptide exhibiting identity over a length of at least about 12 amino acids to SEQ ID NO: 21 or 23;
n) an isolated natural sequence MD2 of mature SEQ ID NO: 21 or 23;
o) a fusion protein comprising primate MD2 sequence;
p) a substantially pure or recombinant MD2 polypeptide exhibiting identity over a length of at least about 12 amino acids to SEQ ID NO: 25 or 26;
q) an isolated natural sequence MD2 of mature SEQ ID NO: 25 or 26; or
r) a fusion protein comprising murine MD2 sequence.

2. The composition of claim 1, which is a substantially pure or isolated:
a) a HCC5 polypeptide, wherein said length is at least 17 amino acids;
b) a Dub11 polypeptide, wherein said length is at least 17 amino acids;
c) a Dub12 polypeptide, wherein said length is at least 17 amino acids;
d) a primate MD1 polypeptide, wherein said length is at least 17 amino acids;
e) a primate MD2 polypeptide, wherein said length is at least 17 amino acids; or
f) a rodent MD2 polypeptide, wherein said length is at least 17 amino acids.

3. The composition of claim 2, which is a substantially pure or isolated:
a) a HCC5 polypeptide, wherein said length is at least 21 amino acids;
b) a Dub11 polypeptide, wherein said length is at least 21 amino acids;
c) a Dub12 polypeptide, wherein said length is at least 21 amino acids;
d) a primate MD1 polypeptide, wherein said length is at least 21 amino acids;
e) a primate MD2 polypeptide, wherein said length is at least 21 amino acids; and
f) a rodent MD2 polypeptide, wherein said length is at least 21 amino acids.

4. The composition of matter of claim 1, wherein said:
a) HCC5 polypeptide:
i) is from a primate, including a human;
ii) comprises at least one polypeptide segment of SEQ ID NO: 2;
iii) exhibits a plurality of portions exhibiting said identity;
iv) is a natural allelic variant of HCC5;
v) has a length at least about 30 amino acids;
vi) exhibits at least two non-overlapping epitopes which are specific for a primate HCC5;
vii) exhibits a sequence identity over a length of at least 35 amino acids to a HCC5;
viii) is glycosylated;
ix) is a synthetic polypeptide;
x) is attached to a solid substrate;
xi) is conjugated to another chemical moiety;
xii) is a 5-fold or less substitution from natural sequence; or
xiii) is a deletion or insertion variant from a natural sequence;
b) Dub11 polypeptide:
i) is from a primate, including a human;
ii) comprises at least one polypeptide segment of SEQ ID NO: 9 or 11;
iii) exhibits a plurality of portions exhibiting said identity;
iv) is a natural allelic variant of Dub11;
v) has a length at least about 30 amino acids;
vi) exhibits at least two non-overlapping epitopes which are specific for a primate Dub11;
vii) exhibits a sequence identity over a length of at least about 35 amino acids to a Dub11;
viii) is glycosylated;
ix) is a synthetic polypeptide;
x) is attached to a solid substrate;
xi) is conjugated to another chemical moiety;
xii) is a 5-fold or less substitution from natural sequence; or
xiii) is a deletion or insertion variant from a natural sequence;
c) Dub12 polypeptide:
i) is from a primate, including a human;
ii) comprises at least one polypeptide segment of SEQ ID NO: 13 or 15;
iii) exhibits a plurality of portions exhibiting said identity;
iv) is a natural allelic variant of Dub12;
v) has a length at least about 30 amino acids;
vi) exhibits at least two non-overlapping epitopes which are specific for a primate Dub12;
vii) exhibits a sequence identity over a length of at least about 35 amino acids to a Dub12;
viii) is glycosylated;
ix) is a synthetic polypeptide;
x) is attached to a solid substrate;
xi) is conjugated to another chemical moiety;
xii) is a 5-fold or less substitution from natural sequence; or
xiii) is a deletion or insertion variant from a natural sequence;
d) primate MD1 polypeptide:
i) is from a human;
ii) comprises at least one polypeptide segment of SEQ ID NO: 19;
iii) exhibits a plurality of portions exhibiting said identity;
iv) is a natural allelic variant of primate MD1;
v) has a length at least about 30 amino acids;
vi) exhibits at least two non-overlapping epitopes which are specific for a primate MD1;
vii) exhibits a sequence identity over a length of at least about 35 amino acids to a primate MD1;
viii) is glycosylated;
ix) is a synthetic polypeptide;
x) is attached to a solid substrate;
xi) is conjugated to another chemical moiety;
xii) is a 5-fold or less substitution from natural sequence; or
xiii) is a deletion or insertion variant from a natural sequence;
e) primate MD2 polypeptide::
i) is from a human;
ii) comprises at least one polypeptide segment of SEQ ID NO: 21 or 23;
iii) exhibits a plurality of portions exhibiting said identity;
iv) is a natural allelic variant of primate MD2;
v) has a length at least about 30 amino acids;
vi) exhibits at least two non-overlapping epitopes which are specific for a primate MD2;
vii) exhibits a sequence identity over a length of at least about 35 amino acids to a primate MD2;
viii) is glycosylated;
ix) is a synthetic polypeptide;
x) is attached to a solid substrate;
xi) is conjugated to another chemical moiety;
xii) is a 5-fold or less substitution from natural sequence; or
xiii) is a deletion or insertion variant from a natural sequence; or
f) rodent MD2 polypeptide:
i) is from a mouse;
ii) comprises at least one polypeptide segment of SEQ ID NO: 25 or 26;
iii) exhibits a plurality of portions exhibiting said identity;
iv) is a natural allelic variant of rodent MD2;
v) has a length at least about 30 amino acids;
vi) exhibits at least two non-overlapping epitopes which are specific for a rodent MD2;
vii) exhibits a sequence identity over a length of at least about 35 amino acids to a rodent MD2;
viii) is glycosylated;
ix) is a synthetic polypeptide;
x) is attached to a solid substrate;
xi) is conjugated to another chemical moiety;
xii) is a 5-fold or less substitution from natural sequence; or
xiii) is a deletion or insertion variant from a natural sequence.

5. A composition comprising a sterile polypeptide of claim 1, wherein said polypeptide is:
a) HCC5 polypeptide;
b) Dub11 polypeptide;
c) Dub12 polypeptide;
d) MD1 polypeptide; or
e) MD2 polypeptide.

6. A composition of claim 1 comprising:
a) said HCC5 polypeptide and:
1) a carrier, wherein said carrier is:
a) an aqueous compound, including water, saline, and/or buffer; and/or
b) formulated for oral, rectal, nasal, topical, or parenteral administration;
2) another chemokine, including one selected from the group of HCC1, HCC2, HCC3, and HCC4; or
3) an antibody antagonist for a chemokine, including one selected from the group of HCC1, HCC2, HCC3, and HCC4;
b) said Dub11 polypeptide and a carrier, wherein said carrier is
a) an aqueous compound, including water, saline, and/or buffer; and/or
b) formulated for oral, rectal, nasal, topical, or parenteral administration;
c) said Dub12 polypeptide and a carrier, wherein said carrier is:
a) an aqueous compound, including water, saline, and/or buffer; and/or
b) formulated for oral, rectal, nasal, topical, or parenteral administration;
d) said MD1 polypeptide and a carrier, wherein said carrier is:
a) an aqueous compound, including water, saline, and/or buffer; and/or
b) formulated for oral, rectal, nasal, topical, or parenteral administration;
e) said MD2 polypeptide and a carrier, wherein said carrier is:
a) an aqueous compound, including water, saline, and/or buffer; and/or
b) formulated for oral, rectal, nasal, topical, or parenteral administration.

7. The fusion protein of claim 1 comprising:
a) mature protein sequence of Table 1;
b) mature protein sequence of Table 2;
b) mature protein sequence of Table 3;
c) a detection or purification tag, including a FLAG, His6, or Ig sequence; or
d) sequence of another chemokine protein with said protein in a).

8. A kit comprising a polypeptide of claim 1, and:
a) a compartment comprising said polypeptide; and/or
b) instructions for use or disposal of reagents in said kit.

9. A binding compound comprising an antigen binding portion from an antibody, which specifically binds to a natural:
a) HCC5 polypeptide of claim 1, wherein said antibody:
i) is raised against a peptide sequence of a mature polypeptide sequence of Table 1;
ii) is raised against a mature HCC5;
iii) is raised to a purified HCC5;
iv) is immunoselected;
v) is a polyclonal antibody;
vi) binds to a denatured HCC5; or
vii) exhibits a Kd to antigen of at least 30 μM;
b) Dub11 polypeptide of claim 1, wherein said antibody:
i) is raised against a peptide sequence of a mature polypeptide sequence of Table 2;
ii) is raised against a mature Dub11;
iii) is raised to a purified Dub11;
iv) is immunoselected;
v) is a polyclonal antibody;
vi) binds to a denatured Dub11; or
vii) exhibits a Kd to antigen of at least 30 μM;
c) Dub12 polypeptide of claim 1, wherein said antibody:
i) is raised against a peptide sequence of a mature polypeptide sequence of Table 2;
ii) is raised against a mature Dub12;
iii) is raised to a purified Dub12;
iv) is immunoselected;
v) is a polyclonal antibody;
vi) binds to a denatured Dub12; or
vii) exhibits a Kd to antigen of at least 30 μM;
d) a primate MD1 polypeptide of claim 1, wherein said antibody:
i) is raised against a peptide sequence of a mature polypeptide sequence of Table 3;
ii) is raised against a mature MD1;
iii) is raised to a purified MD1;
iv) is immunoselected;
v) is a polyclonal antibody;
vi) binds to a denatured MD1; or
vii) exhibits a Kd to antigen of at least 30 μM;
e) a primate MD2 polypeptide of claim 1, wherein said antibody:
i) is raised against a peptide sequence of a mature polypeptide sequence of Table 3;
ii) is raised against a mature MD2;
iii) is raised to a purified MD2;
iv) is immunoselected;
v) is a polyclonal antibody;
vi) binds to a denatured MD2; or
vii) exhibits a Kd to antigen of at least 30 μM; or
f) a rodent MD2 polypeptide of claim 1, wherein said antibody:
i) is raised against a peptide sequence of a mature polypeptide sequence of Table 3;
ii) is raised against a mature rodent MD2;
iii) is raised to a purified rodent MD2;
iv) is immunoselected;
v) is a polyclonal antibody;
vi) binds to a denatured rodent MD2; or
vii) exhibits a Kd to antigen of at least 30 μM.

10. The binding composition of claim 9, wherein:
a) said polypeptide is from a primate or rodent;
b) said binding compound is an Fv, Fab, or Fab2 fragment;
c) said binding compound is conjugated to another chemical moiety;
d) is attached to a solid substrate, including a bead or plastic membrane;
e) is in a sterile composition; or
f) is detectably labeled, including a radioactive or fluorescent label.

11. A kit comprising said binding compound of claim 9, and:
a) a compartment comprising said binding compound;
b) a compartment comprising purified antigen; and/or
c) instructions for use or disposal of reagents in said kit.

12. A method of producing an antigen:antibody complex, comprising contacting an antibody of claim 9 and:
a) a primate HCC5 polypeptide;
b) a primate Dub11 polypeptide;
c) a primate Dub12 polypeptide;
d) a primate MD1 polypeptide;
e) a primate MD2 polypeptide; or
f) a rodent MD2 polypeptide; thereby allowing said complex to form.

13. A composition comprising said binding compound of claim 9 and:
1) a carrier, wherein said carrier is:
a) an aqueous compound, including water, saline, and/or buffer; and/or
b) formulated for oral, rectal, nasal, topical, or parenteral administration; or
2) an antibody antagonist for another chemokine, including one selected from the group of HCC1, HCC2, HCC3, and HCC4.

14. An isolated or recombinant nucleic acid encoding a polypeptide or fusion protein of claim 1, wherein:
A) said HCC5:
a) polypeptide is from a primate, including a human; or
b) nucleic acid:
i) encodes an antigenic peptide sequence of Table 1;
ii) encodes a plurality of antigenic peptide sequences of Table 1;
iii) exhibits identity over at least 25 nucleotides to a natural cDNA encoding said segment;
iv) is a hybridization probe for a gene encoding said HCC5 polypeptide; or
v) further encodes another chemokine, including one selected from the group of HCC1, HCC2, HCC3, and HCC4;
B) said Dub11:
a) polypeptide is from a primate, including a human; or
b) nucleic acid:
i) encodes an antigenic peptide sequence of Table 2;
ii) encodes a plurality of antigenic peptide sequences of Table 2;
iii) exhibits identity over at least 25 nucleotides to a natural cDNA encoding said segment; or
iv) is a hybridization probe for a gene encoding said Dub11 polypeptide;
C) said Dub12:
a) polypeptide is from a primate, including a human; or
b) nucleic acid:
i) encodes an antigenic peptide sequence of Table 2;
ii) encodes a plurality of antigenic peptide sequences of Table 2;
iii) exhibits identity over at least 25 nucleotides to a natural cDNA encoding said segment;
iv) is a hybridization probe for a gene encoding said Dub11 polypeptide;
D) said primate MD1:
a) polypeptide is from a primate, including a human; or
b) nucleic acid:
i) encodes an antigenic peptide sequence of Table 3;
ii) encodes a plurality of antigenic peptide sequences of Table 3;
iii) exhibits identity over at least 25 nucleotides to a natural cDNA encoding said segment;
iv) is a hybridization probe for a gene encoding said Dub11 polypeptide;
E) said primate MD2:
a) polypeptide is from a human; or
b) nucleic acid:
i) encodes an antigenic peptide sequence of Table 3;
ii) encodes a plurality of antigenic peptide sequences of Table 3;
iii) exhibits identity over at least 25 nucleotides to a natural cDNA encoding said segment;
iv) is a hybridization probe for, a gene encoding said primate MD2 polypeptide;
F) said rodent MD2:
a) polypeptide is from a mouse; or
b) nucleic acid:
i) encodes an antigenic peptide sequence of Table 3;
ii) encodes a plurality of antigenic peptide sequences of Table 3;
iii) exhibits identity over at least 25 nucleotides to a natural cDNA encoding said segment; or
iv) is a hybridization probe for a gene encoding said rodent MD2 polypeptide.

15. The nucleic acid of claim 14, which:
a) is an expression vector;
b) further comprises an origin of replication;
c) is from a natural source;
d) comprises a detectable label;
e) comprises synthetic nucleotide sequence;
f) is less than 6 kb, preferably less than 3 kb;
g) is from a primate, including a human;
h) comprises a natural full length coding sequence; or
i) is a PCR primer, PCR product, or mutagenesis primer.

16. A cell or tissue comprising a recombinant nucleic acid of claim 14, including wherein said cell is:
a) a prokaryotic cell;
b) a eukaryotic cell;
c) a bacterial cell;
d) a yeast cell;
e) an insect cell;
f) a mammalian cell;
g) a mouse cell;
h) a primate cell; or
i) a human cell.

17. A kit comprising said nucleic acid of claim 14, and:
a) a compartment comprising said nucleic acid;
b) a compartment comprising a nucleic acid encoding another chemokine, including HCC1, HCC2, HCC3, and HCC4; or
c) instructions for use or disposal of reagents in said kit.

18. A nucleic acid which:
a) hybridizes under wash conditions of 45° C. and less than 2M salt to the coding portion of SEQ ID NO: 1;
b) hybridizes under wash conditions of 45° C. and less than 2M salt to the coding portions of SEQ ID NO: 8 or 10;
c) hybridizes under wash conditions of 45° C. and less than 2M salt to the coding portions of SEQ ID NO: 12, or 14;
d) hybridizes under wash conditions of 45° C. and less than 2M salt to the coding portion of SEQ ID NO: 18;
e) hybridizes under wash conditions of 45° C. and less than 2M salt to the coding portion of SEQ ID NO: 20 or 22;
f) hybridizes under wash conditions of 450 C and less than 2M salt to the coding portion of SEQ ID NO: 24.

19. The nucleic acid of claim 15, wherein:
a) said wash conditions are at 55° C. and/or 500 mM salt; or
b) said wash conditions are at 65° C. and/or 150 mM salt.

20. A method of modulating physiology or development of a cell or tissue culture cells comprising exposing said cell to an agonist or antagonist of HCC5, primate MD1, primate MD2, or rodent MD2.

21. A method of detecting specific binding to a compound, comprising:
a) contacting said compound to a composition selected from the group of:
i) an antigen binding site which specifically binds to a HCC5 chemokine;
ii) an antigen binding site which specifically binds to Dub11;
iii) an antigen binding site which specifically binds to Dub12;
iv) an antigen binding site which specifically binds to primate MD1;
v) an antigen binding site which specifically binds to primate MD2;
vi) an antigen binding site which specifically binds to rodent MD2;
vii) an expression vector encoding a HCC5 chemokine or fragment thereof;
viii) an expression vector encoding a Dub11 or fragment thereof;
ix) an expression vector encoding a Dub12 or fragment thereof;
x) an expression vector encoding a primate MD1 or fragment thereof;
xi) an expression vector encoding a primate MD2 or fragment thereof;
xii) an expression vector encoding a rodent MD2 or fragment thereof;
xiii) a substantially pure protein which is specifically recognized by said antigen binding site of (i);
xiv) a substantially pure protein which is specifically recognized by said antigen binding site of (ii);
xiv) a substantially pure protein which is specifically recognized by said antigen binding site of (iii);
xiv) a substantially pure protein which is specifically recognized by said antigen binding site of (iv);
xiv) a substantially pure protein which is specifically recognized by said antigen binding site of (v);
xiv) a substantially pure protein which is specifically recognized by said antigen binding site of (vi);
ix) a substantially pure HCC5 chemokine or peptide thereof of claim 1, or a fusion protein comprising a 30 amino acid sequence portion of HCC5 chemokine sequence;
x) a substantially pure Dub11 or peptide thereof of claim 1, or a fusion protein comprising a 30 amino acid sequence portion of Dub11 sequence;
xi) a substantially pure Dub12 or peptide thereof of claim 1, or a fusion protein comprising a 30 amino acid sequence portion of Dub11 sequence;
xi) a substantially pure primate MD1 or peptide thereof of claim 1, or a fusion protein comprising a 30 amino acid sequence portion of primate MD1 sequence;
xi) a substantially pure primate MD2 or peptide thereof of claim 1, or a fusion protein comprising a 30 amino acid sequence portion of primate MD2 sequence;
xi) a substantially pure rodent MD2 or peptide thereof of claim 1, or a fusion protein comprising a 30 amino acid sequence portion of rodent MD2 sequence;
b) detecting binding of said compound to said composition.
